(19) United States
(12) Patent Application Publication
(10) Pub. No.: US 2025/0346652 A1
(43) Pub. Date: Nov. 13, 2025

(54) METHODS FOR TREATING DIABETIC KIDNEY DISEASE AND GLOMERULAR DISEASE

(71) Applicant: Nephris SrL, Milano (IT)

(72) Inventors: Paolo FIORINA, Boston, MA (US); Francesca D'ADDIO, Milano (IT)

(21) Appl. No.: 18/292,826

(22) PCT Filed: Jul. 27, 2022
(86) PCT No.: PCT/EP2022/071139

§ 371 Date: Jan. 26, 2024

Related U.S. Application Data
(60) Provisional application No. 63/226,125, filed on Jul. 27, 2021.
(60) Provisional application No. 63/302,460, filed on Jan. 24, 2022.

(51) Int. Cl. C07K 16/18; A61K 39/00

(52) U.S. Cl. CPC C07K 16/18; A61K 2039/505; C07K 2317/55; C07K 2317/71; C07K 2317/76; C07K 2317/92

(57) ABSTRACT

Methods are provided for delaying onset or progression of kidney damage, or treating kidney disease, in a subject who has type 1 diabetes or type 2 diabetes or glomerular disease. The methods comprise administering to a subject with type 1 or type 2 diabetes or glomerular disease an effective amount of an agent capable of inhibiting NBL1 activity, and in particular, capable of inhibiting NBL1-mediated toxicity of human podocytes. In some embodiments, the agent is an antibody capable of binding to human NBL1.

### 1. CROSS-REFERENCE TO RELATED APPLICATIONS

This application is the National Stage of International Application No. PCT/EP2022/071139, filed on Jul. 27, 2022, which claims the benefit of and priority to U.S. Provisional Application No. 63/226,125, filed on Jul. 27, 2021, and 63/302,460, filed on Jan. 24, 2022, which are incorporated herein by reference in their entireties for all purposes.

### 1.1. SEQUENCE LISTING

The instant application contains a Sequence Listing which has been submitted electronically via Patent Center and is hereby incorporated by reference in its entirety. Said XML copy, created on Aug. 20, 2024, is named 49199US_Sequencelisting.xml, and is 601,375 bytes in size.

### 2. BACKGROUND OF INVENTION

Diabetes mellitus type 1 (TID) and type 2 (T2D) collectively affect at least 347 million people worldwide, and prevalence is increasing. Diabetes is characterized by hyperglycemia and complications that greatly impact patient quality and duration of life, placing a major economic burden on society. Research conducted by the American Diabetes Association placed the national economic burden of diabetes in the USA in 2017 at $327 billion. This represents a 26% increase from 245 billion in 2012 when the cost was last examined.

There is no current cure for TID or T2D. Most therapies help patients manage the symptoms to a certain extent, but diabetics still face multiple long-term health complications. Among these complications is kidney damage, which can progress to end-stage renal disease (ESRD). Diabetes was the primary cause of kidney failure in 44% of all new cases in 2011. Given the prevalence and severity of complications associated with diabetes, in particular kidney disease and its progression, there is a need for therapeutic agents that delay onset and progression of kidney disease in diabetes.

Kidney damage also has other etiologies. There is a need for therapeutic agents that delay onset and progression of kidney disease caused by disorders other than diabetes. There is a particular need for therapeutic agents that treat glomerular disorders.

### 3. SUMMARY OF THE INVENTION

As detailed in the experimental examples in this disclosure, we have discovered that the protein, neuroblastoma suppressor of tumorgenicity 1 (NBL1), is directly toxic to renal cells, including podocytes and tubular cells. The toxic effect is not mediated through inhibition of renal BMP proteins; we show that BMPs are not expressed in and are not secreted by kidney cells. Moreover, we have discovered that NBL1 is also not expressed in kidney cells, but is expressed in circulating immune cells. Neutralizing NBL1 with an antagonist, either sBMP2 or anti-NBL1 monoclonal antibodies, prevents toxicity in vitro. Finally, we demonstrate that NBL1 is elevated in Type 1 Diabetes and Type 2 Diabetes. Inhibition of NBL1 is therefore a new therapeutic approach for preventing onset and progression of kidney damage caused by circulating NBL1, and in particular, in a patient with type 1 or type 2 diabetes. Inhibition of NBL1 will also be effective in treating non-diabetes glomerular diseases in which damage is mediated by NBL1.

Accordingly, in a first aspect, methods are provided for delaying onset or progression of kidney damage in a subject who has type 1 diabetes or type 2 diabetes or a glomerular disease. The method comprises administering to the subject an effective amount of an agent capable of inhibiting NBL1 activity, in particular, capable of inhibiting NBL1 toxicity on human podocytes. In some embodiments, the method prevents onset of or slows decline in kidney function.

In a further aspect, methods are provided for slowing decline in kidney function in a subject who has type 1 or type 2 diabetes or a glomerular disease. The method comprises administering to the subject an effective amount of an agent capable of inhibiting NBL1 activity, in particular, capable of inhibiting NBL1 toxicity on human podocytes.

In a yet further aspect, methods are provided for treating diabetic kidney disease (DKD) in a subject who has type 1 or type 2 diabetes or a glomerular disease. The method comprises administering to the subject an effective amount of an agent capable of inhibiting NBL1 activity, in particular, capable of inhibiting NBL1 toxicity on human podocytes.

In some embodiments of these methods, the agent is capable of binding to NBL1. In some embodiments the agent is capable of binding to human NBL1.

In some embodiments the agent is an antibody, or an antigen binding fragment of an antibody, that is capable of binding to human NBL1.

In some embodiments the antibody or antigen-binding fragment comprises three heavy chain CDRs and three light chain CDRs having sequences selected from:
a) SEQ ID NOs: 3, 8, and 13 and SEQ ID NOs: 18, 23, and 28 (antibody E05a);
b) SEQ ID NOs: 33, 38, and 43 and SEQ ID NOs: 48, 53, and 58 (antibody H08);
c) SED ID NOs: 63, 68, and 73 and SED ID NOs: 78, 83, and 88 (antibody F06);
d) SEQ ID NOs: 93, 98, and 103 and SEQ ID NOs: 108, 113, and 118 (antibody A12);
e) SED ID NOs: 123, 128, and 133 and SED ID NOs: 138, 143, and 148 (antibody G01);
f) SED ID NOs: 153, 158, and 163 and SED ID NOs: 168, 173 and 178 (antibody E11);
g) SEQ ID NOs: 183, 188, and 193 and SEQ ID NOs: 198, 203, and 208 (antibody B06);
h) SED ID NOs: 213, 218, and 223 and SED ID NOs: 228, 233, and 238 (antibody D12);
i) SED ID NOs: 243, 248, and 253 and SED ID NOs: 258, 263 and 268 (antibody H01);
j) SED ID NOs: 273, 278, and 283 and SED ID NOs: 288, 293, and 298 (antibody C11);
k) SED ID NOs: 303, 308, and 313 and SED ID NOs: 318, 323, and 328 (antibody E05b);
l) SED ID NOs: 333, 338, and 343 and SED ID NOs: 348, 353 and 358 (antibody F10);
m) SED ID NOs: 363, 368 and 373 and SED ID NOs: 378, 383 and 388 (antibody G10);
n) SEQ ID NOs: 393, 398, and 403 and SEQ ID NOs: 408, 413, and 418 (antibody E04);
o) SED ID NOs: 423, 428, and 433 and SED ID NOs: 438, 443, and 448 (antibody E07);
p) SED ID NOs: 453, 458, and 463 and SED ID NOs: 468, 473, and 478 (antibody E12);
q SED ID NOs: 483, 488, and 493 and SED ID NOs: 498, and 503, and 508 (antibody D08);
r) SED ID NOs: 513, 518, and 523 and SED ID NOs: 528, 533, and 538 (antibody E10);
s) SEQ ID NOs: 543, 548, and 553 and SEQ ID NOs: 558, 563, and 568 (antibody D06); and
t) SED ID NOs: 573, 578, and 583 and SED ID NOs: 588, 593, and 598 (antibody E01),
or having sequences that differ from the selected sets of CDR sequences (a)-(t) by at most two conservative amino acid changes in each CDR.

In some embodiments of the methods, the antibody or antigen-binding fragment comprises three heavy chain CDRs and three light chain CDRs with sequences identical to the selected CDRs. In some embodiments the three heavy chain CDRs and three light chain CDRs have sequences selected from:
b) SEQ ID NOs: 33, 38, and 43 and SEQ ID NOs: 48, 53 and 58 (antibody H08);
d) SEQ ID NOs: 93, 98, and 103 and SEQ ID NOs: 108, 113, and 118 (antibody A12);
g) SEQ ID NOs: 183, 188, and 193 and SEQ ID NOs: 198, 203, and 208 (antibody B06);
n) SEQ ID NOs: 393, 398, and 403 and SEQ ID NOs: 408, 413, and 418 (antibody E04);
s) SEQ ID NOs: 543, 548, and 553 and SEQ ID NOs: 558, 563, and 568 (antibody D06); and
t) SEQ ID NOs: 573, 578, and 583 and SEQ ID NOs: 588, 593, and 598 (antibody E01),
or having sequences that differ from the selected sets of CDR sequences b), d), g), n) s), and t) by at most two conservative amino acid changes in each CDR.

In some embodiments the antibody or antigen-binding fragment comprises three heavy chain CDRs and three light chain CDRs with sequences identical to the selected CDRs.

In some embodiments the antibody framework regions are human antibody framework regions.

In some embodiments the antibody is a full length bivalent monospecific monoclonal antibody. In some embodiments the antibody comprises human IgG1, IgG2, or IgG4 heavy chain constant regions. In some embodiments the antibody comprises a human IgG1 constant region. In some embodiments the antibody Fc region has engineered mutations that reduce antibody binding to at least one type of Fc receptor and/or reduce complement fixation. In some embodiments the antibody is a Fab, optionally wherein the Fab is PEGylated. In some embodiments the antibody or antigen binding fragment is further capable of binding to cynomolgus monkey NBL1. In some embodiments the antibody or antigen binding fragment is further capable of binding to mouse NBL1.

In some embodiments the antibody or antigen binding fragment has a binding affinity (K_{D}) for human NBL1 of less than 100 nM. In some embodiments the antibody or antigen binding fragment has a binding affinity (K_{D}) for human NBL1 of less than 10 nM. In some embodiments the antibody or antigen binding fragment has a binding affinity (K_{D}) for human NBL1 of less than 5 nM. In some embodiments the antibody or antigen binding fragment has a binding affinity (K_{D}) for human NBL1 of less than 1 nM.

In some embodiments, the agent comprises a bone morphogenetic protein (BMP) or soluble fragment thereof. In some embodiments the agent comprises a soluble fragment of human BMP-2. In some particular embodiments the agent further comprises a moiety that extends serum half-life. In some embodiments the half-life extension moiety is an antibody Fc domain. In some embodiments the half-life extension moiety is at least one covalently linked polyethylene glycol (PEG) moiety.

In some embodiments the agent is capable of inhibiting dimerization of NBL1.

In some embodiments the agent is capable of inhibiting NBL1 expression.

In some embodiments, the agent is administered parenterally. In some embodiments the agent is administered intravenously. In some embodiments the agent is administered subcutaneously. In some embodiments the agent is administered for at least 3 months. In some embodiments the agent is administered for at least 6 months. In some embodiments the agent is administered for at least 12 months.

In some embodiments the subject has elevated pre-treatment plasma levels of NBL1. In some embodiments the subject has type 1 diabetes. In some embodiments the subject has type 2 diabetes. In some embodiments the subject has a glomerular disease. In some embodiments the subject with glomerular disease does not have type 1 diabetes or type 2 diabetes. In some embodiments the glomerular disease is selected from the group consisting of focal segmental glomerulosclerosis (FSGS), chronic glomerulopathies, hereditary nephritis, and minimal change disease.

In another aspect, antibodies or antigen binding fragments capable of binding to NBL1 and inhibiting NBL1-induced toxicity of human podocytes are provided.

In some embodiments, the antibody or antigen-binding fragment comprises three heavy chain CDRs and three light chain CDRs having sequences selected from:
a) SEQ ID NOs: 3, 8, and 13 and SEQ ID NOs: 18, 23, and 28 (antibody E05a);
b) SEQ ID NOs: 33, 38, and 43 and SEQ ID NOs: 48, 53, and 58 (antibody H08);
c) SED ID NOs: 63, 68, and 73 and SED ID NOs: 78, 83, and 88 (antibody F06);
d) SEQ ID NOs: 93, 98, and 103 and SEQ ID NOs: 108, 113, and 118 (antibody A12);
e) SED ID NOs: 123, 128, and 133 and SED ID NOs: 138, 143, and 148 (antibody G01);
f) SED ID NOs: 153, 158, and 163 and SED ID NOs: 168, 173 and 178 (antibody E11);
g) SEQ ID NOs: 183, 188, and 193 and SEQ ID NOs: 198, 203, and 208 (antibody B06);
h) SED ID NOs: 213, 218, and 223 and SED ID NOs: 228, 233, and 238 (antibody D12);
i) SED ID NOs: 243, 248, and 253 and SED ID NOs: 258, 263 and 268 (antibody H01);
j) SED ID NOs: 273, 278, and 283 and SED ID NOs: 288, 293, and 298 (antibody C11);
k) SED ID NOs: 303, 308, and 313 and SED ID NOs: 318, 323, and 328 (antibody E05b);
l) SED ID NOs: 333, 338, and 343 and SED ID NOs: 348, 353 and 358 (antibody F10);
m) SED ID NOs: 363, 368 and 373 and SED ID NOs: 378, 383 and 388 (antibody G10);
n) SEQ ID NOs: 393, 398, and 403 and SEQ ID NOs: 408, 413, and 418 (antibody E04);
o) SED ID NOs: 423, 428, and 433 and SED ID NOs: 438, 443, and 448 (antibody E07);
p) SED ID NOs: 453, 458, and 463 and SED ID NOs: 468, 473, and 478 (antibody E12);
q) SED ID NOs: 483, 488, and 493 and SED ID NOs: 498, and 503, and 508 (antibody D08);
r) SED ID NOs: 513, 518, and 523 and SED ID NOs: 528, 533, and 538 (antibody E10);
s) SEQ ID NOs: 543, 548, and 553 and SEQ ID NOs: 558, 563, and 568 (antibody D06); and
t) SED ID NOs: 573, 578, and 583 and SED ID NOs: 588, 593, and 598 (antibody E01),
or having sequences that differ from the CDR sequences selected from (a)-(t) by at most two conservative amino acid substitutions in each CDR. In some embodiments, the antibody or antigen-binding fragment comprises three heavy chain CDRs and three light chain CDRs with sequences identical to one of the selected sets of CDRs (a)-(t).

In some embodiments, the three heavy chain CDRs and three light chain CDRs have sequences selected from:
b) SEQ ID NOs: 33, 38, and 43 and SEQ ID NOs: 48, 53 and 58 (antibody H08);
d) SEQ ID NOs: 93, 98, and 103 and SEQ ID NOs: 108, 113, and 118 (antibody A12);
g) SEQ ID NOs: 183, 188, and 193 and SEQ ID NOs: 198, 203, and 208 (antibody B06);
n) SEQ ID NOs: 393, 398, and 403 and SEQ ID NOs: 408, 413, and 418 (antibody E04);
s) SEQ ID NOs: 543, 548, and 553 and SEQ ID NOs: 558, 563, and 568 (antibody D06); and
t) SEQ ID NOs: 573, 578, and 583 and SEQ ID NOs: 588, 593, and 598 (antibody E01),
or having sequences that differ from the selected CDR sequences b), d), g), n) s), and t) by at most two conservative amino acid substitutions in each CDR. In some embodiments, the antibody or antigen-binding fragment comprises three heavy chain CDRs and three light chain CDRs with sequences identical to the selected CDRs b), d), g), n), s), or t).

In some embodiments, the antibody or antigen-binding fragment framework regions are human antibody framework regions.

In some embodiments, the antibody or antigen binding fragment is a full length bivalent monospecific monoclonal antibody. In some embodiments, the antibody comprises human IgG1, IgG2, or IgG4 heavy chain constant regions. In some embodiments, the antibody comprises a human IgG1 constant region. In some embodiments, the antibody Fc region has engineered mutations that reduce antibody binding to FcRγ and/or reduce complement fixation.

In some embodiments, the antigen binding fragment is a Fab, optionally wherein the Fab is PEGylated.

In some embodiments, the antibody or antigen binding fragment is further capable of binding to cynomolgus monkey NBL1. In some embodiments, the antibody or antigen binding fragment is further capable of binding to mouse NBL1.

In some embodiments, the antibody or antigen binding fragment has a binding affinity (K_{D}) for human NBL1 of less than 100 nM, 10 nM, 5 nM or 1 nM.

In another aspect, pharmaceutical compositions are provided. The compositions comprise the anti-NBL1 antibody or antigen binding fragment, and a pharmaceutically acceptable carrier. In some embodiments, the composition is formulated for parenteral administration. In some embodiments, the composition is formulated for intravenous administration. In some embodiments, the composition is formulated for subcutaneous administration.

### 4. BRIEF DESCRIPTION OF THE SEVERAL VIEWS OF THE DRAWINGS

These and other features, aspects, and advantages of the present invention will become better understood with regard to the following description, and accompanying drawings, where:

FIGS. 1A-1D are bar graphs summarizing cell death analysis in human kidney cells. FIG. 1A shows data from human podocytes (HuPodo) cultured with escalating doses of recombinant human NBL1, from 0.2 μg/ml to 2 μg/ml. FIG. 1B shows data from human mesangial cells (HuMRC) cultured in the presence of 2 μg/ml recombinant human NBL1. FIG. 1C shows data from human renal tubular cells (HuK2) cultured in the presence of 2 μg/ml recombinant human NBL1. FIG. 1D shows data from control human umbilical vein endothelial cells (Huvec) cultured in the presence of 2 μg/ml recombinant human NBL1.

FIGS. 2A-2B depict representative images of confocal analysis conducted on human podocytes cultured with NBL1 at 2 μg/ml (FIG. 2A) or left untreated (FIG. 2B). The podocytes were stained with Apoptag, a marker for apoptosis, and Synaptopodin, a marker for podocytes. FIG. 2C is a bar graph quantifying the percentage of human podocytes that are double positive for Apoptag and Synaptopodin (Syn⁺Apo⁺). The experiment demonstrates NBL1-induced apoptosis of differentiated human podocytes (arrows in FIG. 2B, quantified in FIG. 2C).

FIG. 3 depicts changes in levels of apoptosis-related transcripts in human podocytes cultured with NBL1 at 2 μg/ml as compared to untreated cells.

FIG. 4 is a bar graph quantifying expression of NBL1 mRNA in human immune cells and cell lines. mRNA levels were normalized to beta-actin level.

FIGS. 5A-5B depict the high expression of NBL1 protein in T cells and various T cell subsets (CD3⁺, CD4⁺ and CD8⁺ T cells) and in myeloid cells (CD14⁺) using flow-cytometry analysis. The upper left panel of FIG. 5A depicts physical gating on monocytes (upper gate) and lymphocytes (lower gate). All analysis was conducted on peripheral blood mononuclear cells (PBMCs) isolated from blood samples of healthy volunteers. FIGS. 5C-5D depict the results of an NBL1 Atlas study surveying NBL1 expression in various human tissues by immunohistochemistry. FIG. 5C shows images of various tissue samples illustrating the presence of NBL1 through immunohistochemistry techniques. FIG. 5D is a bar graphing showing high expression of NBL1 protein in intestinal and muscular tissues.

FIG. 6 depicts the neutralizing effect of soluble BMP2 on NBL1-mediated apoptosis. Human podocytes were cultured in the presence of NBL1 (2 μg/ml) and in the presence/absence of soluble BMP2 (1 μg/ml) for 48 hours.

FIGS. 7A-7B depict increased NBL1 serum levels measured by ELISA, with FIG. 7A comparing levels in patients with long-standing type 1 diabetes (TID), type 2 diabetes (T2D), and non-diabetic subjects (CTRL), with FIG. 7B showing NBL1 serum levels in patients with diabetic kidney disease (DKD) stage 2-3 compared to those without DKD.

FIG. 8 are bar graphs quantifying cell death in human podocytes cultured in the presence of NBL1 (2 μg/ml) in the presence and absence of either anti-NBL1 antibodies (20 μg/ml) or soluble BMP2 (1 μg/ml). The left-most bar shows data from cells incubated in medium alone, without NBL1, and without treatment with antibody or sBMP.

### 5. DETAILED DESCRIPTION OF THE INVENTION

### 5.1. Definitions

Unless defined otherwise, all technical and scientific terms used herein have the meaning commonly understood by a person skilled in the art to which this disclosure belongs.

The terms “individual” and “subject” are used interchangeably and refer to an animal to be treated, including but not limited to humans; non-human primates; rodents, including rats and mice; bovines; equines; ovines; felines; and canines.

The term “patient” refers to a human subject.

The terms “treating,” “treatment,” and grammatical variations thereof are used in the broadest sense understood in the clinical arts. Accordingly, the terms do not require cure or complete remission of disease and encompass obtaining any clinically desired pharmacologic and/or physiologic effect. As used herein, “treating diabetic kidney disease (DKD)” explicitly encompasses delaying onset of kidney damage, delaying progression of kidney damage, and slowing decline in kidney function in patients with type 1 or type 2 diabetes or a glomerular disorder.

The phrase “therapeutically effective amount” refers to the amount of a compound that, when administered to a mammal or other subject for treating a disease, condition, or disorder, is sufficient to effect treatment of the disease, condition, or disorder, as treatment is defined herein. Determining the “therapeutically effective amount” is within the skill in the art.

Neuroblastoma suppressor of tumorigenicity 1 (NBL1) is a founding member of the DAN (differential screening-selected gene aberrant in neuroblastoma) gene family. Members of the DAN gene family are expressed during development and function as bone morphogenetic protein (BMP) antagonists; DAN proteins bind to BMPs and prevent them from interacting with BMP receptors. Neuroblastoma suppressor of tumorigenicity 1 (NBL1), also known as DIS1733E, DAN, DAND1, NB, and NO3, is identified by NCBI Gene ID: 4681. The protein sequence of NCBI Gene ID: 4681 is incorporated herein by reference.

Bone morphogenetic proteins (BMPs) are a group of growth factors originally identified by their ability to induce the formation of bone and cartilage, and now considered to constitute a group of pivotal morphogenetic signals, orchestrating tissue architecture throughout the body. Human BMP-2, also known as BDA2, BMP2A, SSFSC, and SSFSC1, is identified by NCBI Gene ID: 650. The protein sequence of NCBI Gene ID: 650 is incorporated herein by reference. Human BMP-4, also known as BMP2B, BMP2B1, MCOPS6, OFC11, and ZYME, is identified by NCBI Gene ID: 652. The protein sequence of NCBI Gene ID: 652 is incorporated herein by reference. Human BMP-7, also known as OP-1, is identified by NCBI Gene ID: 655. The protein sequence of NCBI Gene ID: 655 is incorporated herein by reference.

As used herein, the term “antibody” has its broadest art-recognized meaning, and therefore includes all known formats. The term specifically includes, without limitation, polyclonal antibodies, monoclonal antibodies, monospecific antibodies, multispecific antibodies (including bispecific antibodies), chimeric antibodies, humanized and fully human antibodies. Antibodies that include heavy chain constant region domains can be of any class, including IgG, IgE, IgM, IgD, and IgA and any subclass, including IgG1, IgG2, IgG3, IgG4, IgA1, and IgA2. “Antigen binding fragments” of antibodies are antibody fragments (and/or polypeptides that comprise antibody fragments) that retain the binding characteristics (e.g., specificity, monovalent affinity or bivalent avidity) of the antibody from which derived, and has its broadest art-recognized meaning. The term includes, without limitation, Fab, Fab′, F(ab′)₂, Fv, scFv, (scFv)₂, single domain antibody (including camelid VHH and shark VNAR formats), and multispecific antibodies formed from antibody fragments, including without limitation F(ab)2, and diabody.

As used herein, “chronic kidney disease” (CKD) has the meaning ascribed in the National Kidney Foundation KDOQI guidelines, and stages of CKD are defined as provided in the NKF KDOQI guidelines. “End stage kidney disease” (ESKD) and “end stage renal disease” (ESRD) are used interchangeably herein and have the meaning ascribed in the National Kidney foundation KDOQI guidelines.

In this disclosure, “comprises,” “comprising,” “containing,” “having,” “includes,” “including”, and linguistic variants thereof have the meaning ascribed to them in U.S. Patent law, permitting the presence of additional components beyond those explicitly recited.

Unless specifically stated or apparent from context, as used herein the term “or” is understood to be inclusive.

Unless specifically stated or apparent from context, as used herein, the terms “a”, “an”, and “the” are understood to be singular or plural.

As used herein, “conservative amino acid substitutions” are those substitutions in which the original and substituted amino acids have similar biochemical properties, or their biochemical effects are similarly maintained across substitutions, as set forth in Table 45.

### 5.2. Summary of Experimental Results

As detailed in the experimental examples in this disclosure, we have discovered that NBL1 is directly toxic to renal cells, including podocytes and tubular cells. The toxic effect is not mediated through inhibition of renal BMP proteins; we show that BMPs are not expressed in and are not secreted by kidney cells. Moreover, we have discovered that NBL1 is also not expressed in kidney cells, but is expressed in circulating immune cells. Neutralizing NBL1 with an antagonist prevents toxicity. Finally, we demonstrate that NBL1 is elevated in Type 1 Diabetes and Type 2 Diabetes.

### 5.3. Methods of Delaying Onset or Progression of Kidney Damage

Accordingly, in a first aspect, methods are presented for delaying onset or progression of kidney damage in a subject who has, or is at risk for, type 1 diabetes or type 2 diabetes or who has or is at risk for developing a glomerular disease. The method comprises administering to the subject an effective amount of an agent capable of inhibiting NBL1 activity, and in particular, inhibiting NBL1-mediated toxicity of human podocytes.

In various embodiments, the method delays onset or progression of damage to one or more of renal blood vessels, podocytes, renal tubular cells, or glomerular or tubular basement membrane. In some embodiments, the method delays onset or progression of thickening of glomerular and tubular basement membrane, increase in mesangial matrix, Kimmelstiel-Wilson nodules, microaneurysms, exudative or hyalinosis lesions, capsular drop or afferent and efferent arteriolar hyalinosis.

In some embodiments, the subject has type 1 diabetes. In some embodiments, the subject has type 2 diabetes. In some embodiments, the subject is pre-diabetic. In some embodiments, the subject is not prediabetic as measured by hemoglobin A1c levels or blood glucose levels but is at risk for type 1 diabetes or type 2 diabetes and has elevated NBL1 plasma levels.

In some embodiments, the subject has a glomerular disease. In certain embodiments, the subject with glomerular disease does not have type 1 diabetes or type 2 diabetes. In certain embodiments, the glomerular disease is selected from the group consisting of focal segmental glomerulosclerosis (FSGS), chronic glomerulopathies, hereditary nephritis, and minimal change disease.

In some embodiments, the subject has pre-treatment plasma NBL1 levels at least 10%, 20%, 30%, 40%, 50%, 60%, 70%, 80%, 90% or 100% higher than normal subjects who do not have type 1 or type 2 diabetes or prediabetes. In some embodiments, the subjects to be treated have NBL1 plasma levels at least 2-fold, 3-fold, 4-fold, 5-fold, 6-fold, 7-fold, 8-fold, 9-fold, or 10-fold higher than normal subjects who do not have type 1 or type 2 diabetes or prediabetes.

In some embodiments, the method effectively reduces the availability of circulating free NBL1 in subjects having type 1 or type 2 diabetes, or who are prediabetic, or who are at risk for type 1 diabetes or type 2 diabetes, and have elevated NBL1 plasma levels, as compared to normal subjects who do not have type 1 or type 2 diabetes or prediabetes. In some embodiments, the method effectively reduces the availability of circulating free NBL1 in subjects who have a glomerular disease. In certain embodiments, the method effectively reduces the availability of circulating free NBL1 in subjects who have a glomerular disease selected from the group consisting of focal segmental glomerulosclerosis (FSGS), chronic glomerulopathies, hereditary nephritis, and minimal change disease.

In some embodiments, the agent capable of inhibiting NBL1 activity is capable of binding to NBL1. In some embodiments, the binding occurs in the N-terminus of NBL1. In some embodiments, the binding occurs in the functional DAN domain of NBL1. In some embodiments, the binding occurs in the C-terminus of NBL1. In some embodiments, the binding is noncovalent. In some embodiments, the binding is covalent. In certain covalent embodiments, the inhibitor binds to NBL1 via disulfide bonds within the DAN domain.

In some embodiments, the agent capable of inhibiting NBL1 activity is capable of inhibiting dimerization of NBL1.

In some embodiments, the agent capable of inhibiting NBL1 activity is a dimerization inhibitor that prevents the formation of stabilizing hydrogen bonds between two NBL1 monomers. In some embodiments, the dimerization inhibitor disrupts at least one of the three disulfide bonds that form a ring-like structure known as cysteine-knot motif within each monomer, or the disulfide bond linking F1 to F2, where F1 and F2 are the first loop from the N terminus, finger 1, and the third loop from the N terminus, finger 2, respectively.

### 5.3.1. Soluble BMP Inhibitors

In some embodiments, the agent capable of inhibiting NBL1 activity comprises a bone morphogenetic protein (BMP) or soluble NBL1-binding fragment thereof. In some embodiments, the BMP is BMP-2 or a soluble NBL1-binding fragment thereof. In some embodiments, the BMP is BMP-4 or a soluble NBL1-binding fragment thereof. In some embodiments, the BMP is BMP-7 or a soluble NBL1-binding fragment thereof. In currently preferred embodiments, the BMP is a human BMP or soluble NBL1-binding fragment thereof.

In some embodiments, the agent capable of inhibiting NBL1 further comprises a moiety that extends serum half-life.

In some embodiments, the moiety that extends serum half-life is provided through covalent chemical modification. In some embodiments, the moiety that extends serum half-life is at least one polyethylene glycol (PEG) moiety. In some embodiments, the PEG moiety is permanently attached to the agent. In some embodiments, the PEG moiety is a releasable carrier, provided through covalent chemical modification. In certain embodiments, the agent comprises a PEGylated BMP or soluble NBL1-binding fragment thereof.

In some embodiments, the moiety that extends serum half-life is an antibody Fc domain. In some embodiments, the Fc domain is engineered to optimize the pH-dependent IgG Fc-FcRn interaction. In certain embodiments, the Fc domain is engineered to have the YTE triple mutation (M252Y/S254T/T256E). In certain embodiments, the Fc domain is engineered to have the M428L/N434S mutations. In certain embodiments, the Fc domain is fused in frame to a BMP or soluble NBL1-binding fragment thereof.

In some embodiments, the moiety that extends serum half-life is a serum albumin molecule. In certain embodiments, the half-life extending moiety is a human serum albumin molecule fused in frame to a BMP or soluble NBL1-binding fragment thereof.

In some embodiments, the moiety that extends serum half-life is an XTEN protein polymer covalently attached to the agent, as described in Podust et al., Protein Eng. Des. Sel. 26 (11): 743-53 (2013), the disclosure of which is incorporated herein by reference in its entirety.

### 5.3.2. Antibodies and Antigen-Binding Fragments Thereof

In some embodiments, the agent capable of inhibiting NBL1 activity is an antibody capable of binding NBL1, or an NBL1-binding fragment thereof and inhibiting NBL1 activity, and in particular, inhibiting NBL1-mediated toxicity of human podocytes. In preferred embodiments, the antibody or antigen-binding antibody fragment is capable of binding human NBL1.

In some embodiments, the antibody or antigen-binding fragment thereof binds to an epitope within the N-terminal domain of NBL1. In some embodiments, the antibody or antigen-binding fragment thereof binds to an epitope within the DAN domain of NBL1. In some embodiments, the antibody or antigen-binding fragment thereof binds to an epitope within the C-terminal domain of NBL1.

In some embodiments, the antibody or antigen-binding fragment thereof comprises heavy chain and light chain CDRs selected from Tables 23-44 below, or the heavy chain and light chain CDRs selected from Tables 23-44 below with no more than 10 amino acid deletions, insertions, or conservative amino acid substitutions (as defined in Table 45), as compared thereto.

TABLE 45
Conservative amino acid substitutions

| Class | Amino Acids |
|---|---|
| Aliphatic | Glycine, Alanine, Valine, Leucine, Isoleucine |
| Hydroxyl or sulfur/ | Serine, Cysteine, Selenocysteine, Threonine, |
| selenium containing | Methionine |
| Cyclic | Proline |
| Aromatic | Phenylalanine, Tyrosine, Tryptophan |
| Basic | Histidine, Lysine, Arginine |
| Acidic and their amides | Aspartate, Glutamate, Asparagine, Glutamine |

In some embodiments, the amino acid deletions, insertions, or conservative substitutions are no more than 8. In some embodiments, the amino acid deletions, insertions, or conservative substitutions are no more than 6. In some embodiments, the amino acid deletions, insertions, or conservative substitutions are no more than 4.

In some embodiments, the antibody or antigen-binding fragment thereof comprises heavy chain V region and light chain V region selected from Tables 1-22 below.

In some embodiments, the antibody or antigen-binding fragment thereof comprises the heavy chain and light chain CDRs selected from Tables 23-44 and with no more than 10 amino acid deletions, insertions, or conservative amino acid substitutions (as defined in Table 45), as compared thereto. In some embodiments, the amino acid deletions, insertions, or conservative substitutions are no more than 8. In some embodiments, the amino acid deletions, insertions, or conservative substitutions are no more than 6. In some embodiments, the amino acid deletions, insertions, or conservative substitutions are no more than 4.

TABLE 1
Antibody YU1018-E05a

| | |
|---|---|
| VH | EVQLLESGPGLVKPSQTLSLTCTVSGGSISSGGYYWSWIRQHPGKGLEWIGYIYYSGSTYYNPSLKSRVTISVDTSKNQFSLKLS |
| | SVTAADTAVYYCARTNSRITMIADWGQGTLVTVSS |
| VL | QSVLTQPPSVSAAPGQKVTISCSGSSSNIGNNYVSWYQQLPGTAPKLLIYDNNKRPSGIPDRFSGSKSGTSATLGITGLQTGDEA |
| | DYYCGTWDSSLSAGVFGGGTQLTVL |

TABLE 23
Antibody Y1018-E05a

| CDRs | Chothia | AbM | Kabat | Contact | IMGT |
|---|---|---|---|---|---|
| heavy | GGSISSGGY | GGSISSGGYYWS | SGGYYWS | SSGGYYWS | GGSISSGGYY |
| chain CDR1 | (SEQ ID NO: 1) | (SEQ ID NO: 2) | (SEQ ID NO: 3) | (SEQ ID NO: 4) | (SEQ ID NO: 5) |
| heavy | YYSGS | YIYYSGSTY | YIYYSGSTYYNPSL | WIGYIYYSGSTY | IYYSGST |
| chain CDR2 | (SEQ ID NO: 6) | (SEQ ID NO: 7) | KS | (SEQ ID NO: 9) | (SEQ ID NO: 10) |
| | | | (SEQ ID NO: 8) | | |
| heavy | TNSRITMIAD | TNSRITMIAD | TNSRITMIAD | ARTNSRITMIA | ARTNSRITMIAD |
| chain CDR3 | (SEQ ID NO: 11) | (SEQ ID NO: 12) | (SEQ ID NO: 13) | (SEQ ID NO: 14) | (SEQ ID NO: 15) |
| light | SGSSSNIGNNYVS | SGSSSNIGNNYVS | SGSSSNIGNNYVS | IGNNYVSWY | SSNIGNNY |
| chain CDR1 | (SEQ ID NO: 16) | (SEQ ID NO: 17) | (SEQ ID NO: 18) | (SEQ ID NO: 19) | (SEQ ID NO: 20) |
| light | DNNKRPS | DNNKRPS | DNNKRPS | LLIYDNNKRP | DN |
| chain CDR2 | (SEQ ID NO: 21) | (SEQ ID NO: 22) | (SEQ ID NO: 23) | (SEQ ID NO: 24) | (SEQ ID NO: 25) |
| light | GTWDSSLSAGV | GTWDSSLSAGV | GTWDSSLSAGV | CTWDSSLSAG | GTWDSSLSAGV |
| chain CDR3 | (SEQ ID NO: 26) | (SEQ ID NO: 27) | (SEQ ID NO: 28) | (SEQ ID NO: 29) | (SEQ ID NO: 30) |

TABLE 2
Antibody YU1018-H08

| | |
|---|---|
| VH | EVQLLESGPGLVKPSQTLSLTCTVSGGSISSGGYYWSWIRQHPGKGLEWIGYIYYSGSTYYNPSLKSRVTISVDTSKNQFSLKLSS |
| | VTAADTAVYYCARTNSRITMIADWGQGTLVTVSS |
| VL | QSVLTQPPSVSAAPGQKVTISCSGSSSNIGNNYVSWYQQLPGTAPKLLTYDNNKRPSGIPDRFSGSKSGTSATLGITGLQTGDEAD |
| | YYCGTWDSSLSAGHVFGGGTKVTVL |

TABLE 24
Antibody Y1018-H08
CDRs

| | Chothia | AbM | Kabat | Contact | IMGT |
|---|---|---|---|---|---|
| heavy | GGSISSGGY | GGSISSGGYYWS | SGGYYWS | SSGGYYWS | GGSISSGGYY |
| chain CDR1 | (SEQ ID NO: 31) | (SEQ ID NO: 32) | (SEQ ID NO: 33) | (SEQ ID NO: 34) | 3(SEQ ID NO: 35) |
| heavy | YYSGS | YIYYSGSTY | YIYYSGSTYYNPSLKS | WIGYIYYSGSTY | IYYSGST |
| chain CDR2 | (SEQ ID NO: 36) | (SEQ ID NO: 37) | (SEQ ID NO: 38) | (SEQ ID NO: 39) | (SEQ ID NO: 40) |
| heavy | TNSRITMIAD | TNSRITMIAD | TNSRITMIAD | ARTNSRITMIA | ARTNSRITMIAD |
| chain CDR3 | (SEQ ID NO: 41) | (SEQ ID NO: 42) | (SEQ ID NO: 43) | (SEQ ID NO: 44) | (SEQ ID NO: 45) |
| light | SGSSSNIGNNYVS | SGSSSNIGNNYVS | SGSSSNIGNNYVS | IGNNYVSWY | SSNIGNNY |
| chain CDR1 | (SEQ ID NO: 46) | (SEQ ID NO: 47) | (SEQ ID NO: 48) | (SEQ ID NO: 49) | (SEQ ID NO: 50) |
| light | DNNKRPS | DNNKRPS | DNNKRPS | LLTYDNNKRP | DN |
| chain CDR2 | (SEQ ID NO: 51) | (SEQ ID NO: 52) | (SEQ ID NO: 53) | (SEQ ID NO: 54) | (SEQ ID NO: 55) |
| light | GTWDSSLSAGHV | GTWDSSLSAGHV | GTWDSSLSAGHV | GTWDSSLSAGH | GTWDSSLSAGHV |
| chain CDR3 | (SEQ ID NO: 56) | (SEQ ID NO: 57) | (SEQ ID NO: 58) | (SEQ ID NO: 59) | (SEQ ID NO: 60) |

TABLE 3
Antibody YU1018-F06

| | |
|---|---|
| VH | QVQLVESGGGLVQPGGSLRLSCAASGFTFSSYAMSWVRQAPGKGLEWVSAISGSGGSTYYADSVKGRFTISRDNSKNTLYLQMNSL |
| | RAEDTAVYYCAKGSPYYYDSSGYYPLDYWGQGTLVTVSS |
| VL | DVVMTQSPLSLPVTPGEPASISCRSSQSLLHSNGYNYLDWYLQKPGQSPQLLIYLGSNRAPGVPDRFSGSGSGTDFTLKISRVEAED |
| | VGVYYCMQALQTPLTFGPGTKLEIK |

TABLE 25
Antibody Y1018-F06
CDRs

| | Chothia | AbM | Kabat | Contact | IMGT |
|---|---|---|---|---|---|
| heavy | GFTFSSY | GFTFSSYAMS | SYAMS | SSYAMS | GFTFSSYA |
| chain | (SEQ ID NO: 61) | (SEQ ID NO: 62) | (SEQ ID NO: 63) | (SEQ ID NO: 64) | (SEQ ID NO: 65) |
| CDR1 | | | | | |
| heavy | SGSGGS | AISGSGGSTY | AISGSGGSTYYADSVKG | WVSAISGSGGSTY | ISGSGGST |
| chain | (SEQ ID NO: 66) | (SEQ ID NO: 67) | (SEQ ID NO: 68) | (SEQ ID NO: 69) | (SEQ ID NO: 70) |
| CDR2 | | | | | |
| heavy | GSPYYYDSSGYYPLDY | GSPYYYDSSGYYPLDY | GSPYYYDSSGYYPLDY | AKGSPYYYDSSGYYPLD | AKGSPYYYDSSGYYPLD |
| chain | (SEQ ID NO: 71) | (SEQ ID NO: 72) | (SEQ ID NO: 73) | (SEQ ID NO: 74) | Y (SEQ ID NO: 75) |
| CDR3 | | | | | |
| light | RSSQSLLHSNGYNYLD | RSSQSLLHSNGYNYLD | RSSQSLLHSNGYNYLD | LHSNGYNYLDWY | QSLLHSNGYNY |
| chain | (SEQ ID NO: 76) | (SEQ ID NO: 77) | (SEQ ID NO: 78) | (SEQ ID NO: 79) | (SEQ ID NO: 80) |
| CDR1 | | | | | |
| light | LGSNRAP | LGSNRAP | LGSNRAP | LLIYLGSNRA | LG |
| chain | (SEQ ID NO: 81) | (SEQ ID NO: 82) | (SEQ ID NO: 83) | (SEQ ID NO: 84) | (SEQ ID NO: 85) |
| CDR2 | | | | | |
| light | MQALQTPLT | MQALQTPLT | MQALQTPLT | MQALQTPL | MQALQTPLT |
| chain | (SEQ ID NO: 86) | (SEQ ID NO: 87) | (SEQ ID NO: 88) | (SEQ ID NO: 89) | (SEQ ID NO: 90) |
| CDR3 | | | | | |

TABLE 4
Antibody YU1019-A12

| | |
|---|---|
| VH | EVQLLESGPGLVKPSQTLSLTCTVSGGSISSGDYYWSWIRQPPGKGLEWIGYIYYSGSTYYNPSLKSRVTISVDTSKNQFSLKLS |
| | SVTAADTAVYYCARGRRLLLFHWGQGTTVTVSS |
| VL | QSVLTQPPSVSAAPGQKVTISCSGSGSSIGNNYVSWYQQVPGTPPKLLIYDNNKRASGIPDRFSGSKSGTSATLGITGLQTGDE |
| | ADYYCGTWDNSLSAVVFGGGTKLTVL |

TABLE 26
Antibody YU1019-A12
CDRs

| | Chothia | AbM | Kabat | Contact | IMGT |
|---|---|---|---|---|---|
| heavy | CGSISSGDY | GGSISSGDYYWS | SGDYYWS | SSGDYYWS | GGSISSGDYY |
| chain CDR1 | (SEQ ID NO: 91) | (SEQ ID NO: 92) | (SEQ ID NO: 93) | (SEQ ID NO: 94) | (SEQ ID NO: 95) |
| heavy | YYSGS | YIYYSGSTY | YIYYSGSTYYNPSLKS | WIGYIYYSGSTY | IYYSGST |
| chain CDR2 | (SEQ ID NO: 96) | (SEQ ID NO: 97) | (SEQ ID NO: 98) | (SEQ ID NO: 99) | (SEQ ID NO: 100) |
| heavy | GRRLLLFH | GRRLLLFH | GRRLLLFH | ARGRRLLLF | ARGRRLLLFH |
| chain CDR3 | (SEQ ID NO: 101) | (SEQ ID NO: 102) | (SEQ ID NO: 103) | (SEQ ID NO: 104) | (SEQ ID NO: 105) |
| light | SGSGSSIGNNYVS | SGSGSSIGNNYVS | SGSGSSIGNNYVS | IGNNYVSWY | GSSIGNNY |
| chain CDR1 | (SEQ ID NO: 106) | (SEQ ID NO: 107) | (SEQ ID NO: 108) | (SEQ ID NO: 109) | (SEQ ID NO: 110) |
| light | DNNKRAS | DNNKRAS | DNNKRAS | LLIYDNNKRA | DN |
| chain CDR2 | (SEQ ID NO: 111) | SEQ ID NO: 112) | (SEQ ID NO: 113) | (SEQ ID NO: 114) | (SEQ ID NO: 115) |
| light | CTWDNSLSAVV | GTWDNSLSAVV | GTWDNSLSAVV | GTWDNSLSAV | GTWDNSLSAVV |
| chain CDR3 | (SEQ ID NO: 116) | (SEQ ID NO: 117) | (SEQ ID NO: 118) | (SEQ ID NO: 119) | (SEQ ID NO: 120) |

TABLE 5
Antibody YU1018-G01

| | |
|---|---|
| VH | QVQLVQSGAEVKKPGASVKVSCKASGYTFTSYAMHWVRQAPGQRLEWMGWINAGNGNTKYSQKFQGRVTITRDTSASTAYMELSS |
| | LRSEDTAVYYCARGRELLTFDYWGQGTLVTVSS |
| VL | DVVMTQSPLSLPVTPGEPASISCRSSQSLLHSNGYNYLDWYLQKPGQSPQLLIYLGSNRASGVPDRFSGSGSGTDFTLKISRVEA |
| | EDVGVYYCMQALQTPLTFGGGTKVEIK |

TABLE 27
Antibody YU1018-G01
CDRs

| | Chothia | AbM | Kabat | Contact | IMGT |
|---|---|---|---|---|---|
| heavy chain CDR1 | GYTFTSY | GYTFTSYAMH | SYAMH | TSYAMH | GYTFTSYA |
| | (SEQ ID NO: 121) | (SEQ ID NO: 122) | (SEQ ID NO: 123) | (SEQ ID NO: 124) | (SEQ ID NO: 125) |
| heavy chain CDR2 | NAGNGN | WINAGNGNTK | WINAGNGNIKYSQKF | WMGWINAGNGNTK | INAGNGNT |
| | (SEQ ID NO: 126) | (SEQ ID NO: 127) | QG (SEQ ID NO: 128) | (SEQ ID NO: 129) | (SEQ ID NO: 130) |
| heavy chain CDR3 | GRELLTFDY | GRELLTFDY | GRELLTFDY | ARGRELLTFD | ARGRELLTFDY |
| | (SEQ ID NO: 131) | (SEQ ID NO: 132) | (SEQ ID NO: 133) | (SEQ ID NO: 134) | (SEQ ID NO: 135) |
| light chain CDR1 | RSSQSLLHSNGYNYL | RSSQSLLHSNGYNYLD | RSSQSLLHSNGYNYLD | LHSNGYNYLDWY | QSLLHSNGYNY |
| | D | (SEQ ID NO: 137) | (SEQ ID NO: 138) | (SEQ ID NO: 139) | (SEQ ID NO: 140) |
| | (SEQ ID NO: 136) | | | | |
| light chain CDR2 | LGSNRAS | LGSNRAS | LGSNRAS | LLIYLGSNRA | LG |
| | (SEQ ID NO: 141) | (SEQ ID NO: 142) | (SEQ ID NO: 143) | (SEQ ID NO: 144) | (SEQ ID NO: 145) |
| light chain CDR3 | MQALQTPLT | MQALQTPLT | MQALQTPLT | MQALQTPL | MQALQTPLT |
| | (SEQ ID NO: 146) | (SEQ ID NO: 147) | (SEQ ID NO: 148) | (SEQ ID NO: 149) | (SEQ ID NO: 150) |

TABLE 6
Antibody YU1019-E11

| | |
|---|---|
| VH | QVQLQESGPGLVKPSQTLSLTCTVSGGSISSGGYYWSWIRQHPGKGLEWIGYIYYSGSTYYNPSLKSRVTISVDTSKNQFSLKLSS |
| | VTAADTAVYYCARDAPLRIGAFDIWGQGTMVTVSS |
| VL | QSVLTQPPSVSAAPGQKVTISCSGSSSNIGNNYVSWYQQLPGTAPKLLIYDNNKRPSGIPDRFSGSKSGTSATLGITRLQTGDEA |
| | DYYCGTWDSSLSAGVFGGGTKLTVL |

TABLE 28
Antibody YU1019-E11
CDRs

| | Chothia | AbM | Kabat | Contact | IMGT |
|---|---|---|---|---|---|
| heavy | GGSISSGGY | GGSISSGGYYWS | SGGYYWS | SSGGYYWS | GGSISSGGYY |
| chain CDR1 | (SEQ ID NO: 151) | (SEQ ID NO: 152) | (SEQ ID NO: 153) | (SEQ ID NO: 154) | (SEQ ID NO: 155) |
| heavy | YYSGS | YIYYSGSTY | YIYYSGSTYYNPSLKS | WIGYIYYSGSTY | IYYSGST |
| chain CDR2 | (SEQ ID NO: 156) | (SEQ ID NO: 157) | (SEQ ID NO: 158) | (SEQ ID NO: 159) | (SEQ ID NO: 160) |
| heavy | DAPLRIGAFDI | DAPLRIGAFDI | DAPLRIGAFDI | ARDAPLRIGAFD | ARDAPLRIGAFDI |
| chain CDR3 | (SEQ ID NO: 161) | (SEQ ID NO: 162) | (SEQ ID NO: 163) | (SEQ ID NO: 164) | (SEQ ID NO: 165) |
| light | SGSSSNIGNNYVS | SGSSSNIGNNYVS | SGSSSNIGNNYVS | IGNNYVSWY | SSNIGNNY |
| chain CDR1 | (SEQ ID NO: 166) | (SEQ ID NO: 167) | (SEQ ID NO: 168) | (SEQ ID NO: 169) | (SEQ ID NO: 170) |
| light | DNNKRPS | DNNKRPS | DNNKRPS | LLIYDNNKRP | DN |
| chain CDR2 | (SEQ ID NO: 171) | (SEQ ID NO: 172) | (SEQ ID NO: 173) | (SEQ ID NO: 174) | (SEQ ID NO: 175) |
| light | GTWDSSLSAGV | GTWDSSLSAGV | GTWDSSLSAGV | GTWDSSLSAG | GTWDSSLSAGV |
| chain CDR3 | (SEQ ID NO: 176) | (SEQ ID NO: 177) | (SEQ ID NO: 178) | (SEQ ID NO: 179) | (SEQ ID NO: 180) |

TABLE 7
Antibody YU1019-B06

| | |
|---|---|
| VH | EVQLLESGGGVVQPGRSLRLSCAASGFTFSSYAMHWVRQAPGKGLEWVAVISYDGSNKYYADSVKGRFTISRDNSKNTLYLQMN |
| | SLRAEDTAVYYCASPVLRYFDWLPNYWGQGTLVTVSS |
| VL | DVVMTQSPLSLPVTLGQPASISCRSSQSLVYSDGNTYLNWFQQRPGQSPRRLIYKVSNRDSGVPDRFSGSGSGTDFTLKISRV |
| | EAEDVGVYYCMQGTHWPPAFGPGTKVDIK |

TABLE 29
Antibody YU1019-B06
CDRs

| | Chothia | AbM | Kabat | Contact | IMGT |
|---|---|---|---|---|---|
| heavy chain CDR1 | GFTFSSY | GFTFSSYAMH | SYAMH | SSYAMH | GFTFSSYA |
| | (SEQ ID NO: 181) | (SEQ ID NO: 182) | (SEQ ID NO: 183) | (SEQ ID NO: 184) | (SEQ ID NO: 185) |
| heavy chain CDR2 | SYDGSN | VISYDGSNKY | VISYDGSNKYYADSVK | WVAVISYDGSNKY | ISYDGSNK |
| | (SEQ ID NO: 186) | (SEQ ID NO: 187) | G (SEQ ID NO: 188) | (SEQ ID NO: 189) | (SEQ ID NO: 190) |
| heavy chain CDR3 | PVLRYFDWLPNY | PVLRYFDWLPNY | PVLRYFDWLPNY | ASPVLRYFDWLPN | ASPVLRYFDWLPNY |
| | (SEQ ID NO: 191) | (SEQ ID NO: 192) | (SEQ ID NO: 193) | (SEQ ID NO: 194) | (SEQ ID NO: 195) |
| light chain CDR1 | RSSQSLVYSDGNTYL | RSSQSLVYSDGNTYLN | RSSQSLVYSDGNTYLN | VYSDGNTYLNWF | QSLVYSDGNTY |
| | N (SEQ ID NO: 196) | (SEQ ID NO: 197) | (SEQ ID NO: 198) | (SEQ ID NO: 199) | (SEQ ID NO: 200) |
| light chain CDR2 | KVSNRDS | KVSNRDS | KVSNRDS | RLIYKVSNRD | KV |
| | (SEQ ID NO: 201) | (SEQ ID NO: 202) | (SEQ ID NO: 203) | (SEQ ID NO: 204) | (SEQ ID NO: 205) |
| light chain CDR3 | MQGTHWPPA | MQGTHWPPA | MQGTHWPPA | MQGTHWPP | MQGTHWPPA |
| | (SEQ ID NO: 206) | (SEQ ID NO: 207) | (SEQ ID NO: 208) | (SEQ ID NO: 209) | (SEQ ID NO: 210) |

TABLE 8
Antibody YU1018-D12

| | |
|---|---|
| VH | EVQLVQSGAEVKKPGASVKVSCKASGYTFTSYGISWVRQAPGQGLEWMGWISAYNGNTNYAQKLQGRVTMTTDTSTSTAYMELRS |
| | LRSDDTAVYYCARDVGYSYGIFDYWGQGTLVTVSS |
| VL | DVVMTQSPLSLPATPGEPASISCRSSQSLLHSNGYNYLDWYLQKPGQSPQLLIYLGSNRASGVPDRFSGSGSGTDFTLKISRVEA |
| | EDVGVYYCMQALQTPYTFGQGTKVEIK |

TABLE 30
Antibody YU1018-D12
CDRs

| | Chothia | AbM | Kabat | Contact | IMGT |
|---|---|---|---|---|---|
| heavy chain CDR1 | GYTFTSY | GYTFTSYGIS | SYGIS | TSYGIS | GYTFTSYG |
| | (SEQ ID NO: 211) | (SEQ ID NO: 212) | (SEQ ID NO: 213) | (SEQ ID NO: 214) | (SEQ ID NO: 215) |
| heavy chain CDR2 | SAYNGN | WISAYNGNIN | WISAYNGNTNYAQKLQG | WMGWISAYNGNTN | ISAYNGNT |
| | (SEQ ID NO: 216) | (SEQ ID NO: 217) | (SEQ ID NO: 218) | (SEQ ID NO: 219) | (SEQ ID NO: 220) |
| heavy chain CDR3 | DVOYSYGIFDY | DVGYSYGIFDY | DVGYSYGIFDY | ARDVGYSYGIED | ARDVGYSYGIFDY |
| | (SEQ ID NO: 221) | (SEQ ID NO: 222) | (SEQ ID NO: 223) | (SEQ ID NO: 224) | (SEQ ID NO: 225) |
| light chain CDR1 | RSSQSLLHSNGYNYL | RSSQSLLHSNGYN | RSSQSLLHSNGYNYLD (SEQ | LHSNGYNYLDWY | QSLLHSNGYNY |
| | D | YLD | ID NO: 228) | (SEQ ID NO: 229) | (SEQ ID NO: 230) |
| | (SEQ ID NO: 226) | (SEQ ID NO: 227) | | | |
| light chain CDR2 | LGSNRAS | LGSNRAS | LGSNRAS | LLIYLGSNRA | LG |
| | (SEQ ID NO: 231) | (SEQ ID NO: 232) | (SEQ ID NO: 233) | (SEQ ID NO: 234) | (SEQ ID NO: 235) |
| light chain CDR3 | MQALQTPYT | MQALQTPYT | MQALQTPYT | MQALQTPY | MQALQTPYT |
| | (SEQ ID NO: 236) | (SEQ ID NO: 237) | (SEQ ID NO: 238) | (SEQ ID NO: 239) | (SEQ ID NO: 240) |

TABLE 9
Antibody YU1019-H01

| | |
|---|---|
| VH | EVQLVQSGAEVKKPGASVKVSCKASGYTFTSYYMHWVRQAPGQGLEWMGIINPSGGSTSYAQKFQGRVTMTRDTSTSTVYMELS |
| | SLRSEDTAVYYCARAPGKFPLDYWGQGTLVTVSS |
| VL | DVVMTQSPLSLPVTPGEPASISCRSSQSLLHSNGYNYLDWYLQKPGQSPQLLIYLGSNRASGVPDRFSGSGSGTDFTLKISRV |
| | EAEDVGVYYCMQALQTPRTFGGGTKVEIK |

TABLE 31
Antibody YU1019-H01
CDRs

| | Chothia | AbM | Kabat | Contact | IMGT |
|---|---|---|---|---|---|
| heavy chain CDR1 | GYTFTSY | GYTFTSYYMH | SYYMH | TSYYMH | GYTFTSYY |
| | (SEQ ID NO: 241) | (SEQ ID NO: 242) | (SEQ ID NO: 243) | (SEQ ID NO: 244) | (SEQ ID NO: 245) |
| heavy chain CDR2 | NPSGGS | IINPSGGSTS | IINPSGGSTSYAQKFQG | WMGIINPSGGSTS | INPSGGST |
| | (SEQ ID NO: 246) | (SEQ ID NO: 247) | (SEQ ID NO: 248) | (SEQ ID NO: 249) | (SEQ ID NO: 250) |
| heavy chain CDR3 | APGKFPLDY | APGKFPLDY | APGKFPLDY | ARAPGKFPLD | ARAPGKFPLDY |
| | (SEQ ID NO: 251) | (SEQ ID NO: 252) | (SEQ ID NO: 253) | (SEQ ID NO: 254) | (SEQ ID NO: 255) |
| light chain CDR1 | RSSQSLLHSNGYNYLD | RSSQSLLHSNGYNYLD | RSSQSLLHSNGYNYLD | LHSNGYNYLDWY | QSLLHSNGYNY |
| | (SEQ ID NO: 256) | (SEQ ID NO: 257) | (SEQ ID NO: 258) | (SEQ ID NO: 259) | (SEQ ID NO: 260) |
| light chain CDR2 | LGSNRAS | LGSNRAS | LGSNRAS | LLIYLGSNRA | LG |
| | (SEQ ID NO: 261) | (SEQ ID NO: 262) | (SEQ ID NO: 263) | (SEQ ID NO: 264) | (SEQ ID NO: 265) |
| light chain CDR3 | MQALQTPRT | MQALQTPRI | MQALQTPRT | MQALQTPR | MQALQTPRT |
| | (SEQ ID NO: 266) | (SEQ ID NO: 267) | (SEQ ID NO: 268) | (SEQ ID NO: 269) | (SEQ ID NO: 270) |

TABLE 10
Antibody YU1018-C11

| | |
|---|---|
| VH | QVQLVQSGGGLVQPGGSLRLSCAASGFTFSSYAMSWVRQAPGKGLEWVSAISGSGGSTYYADSVKGRFTISRDNSKNTLYLQMN |
| | SLRAEDTAVYYCARGGFWGSYRYWSAAFDIWGQGTMVTVSS |
| VL | DIVMTQSPLSLPVTPGEPASISCRSSQSLLHSNGYNYLDWYLQKPGQSPQLLIYLGSNRASGVPDRFSGSGSGTDFTLKISRVE |
| | AEDVGVYYCMQALQTPPTFGGGTKVEIK |

TABLE 32
Antibody YU1018-C11
CDRs

| | Chothia | AbM | Kabat | Contact | IMGT |
|---|---|---|---|---|---|
| heavy chain CDR1 | GFTFSSY | GFTFSSYAMS | SYAMS | SSYAMS | GFTFSSYA |
| | (SEQ ID NO: 271) | (SEQ ID NO: 272) | (SEQ ID NO: 273) | (SEQ ID NO: 274) | (SEQ ID NO: 275) |
| heavy chain CDR2 | SGSGGS | AISGSGGSTY | AISGSGGSTYYADSVK | WVSAISGSGGSTY | ISGSGGST |
| | (SEQ ID NO: 276) | (SEQ ID NO: 277) | G | (SEQ ID NO: 279) | (SEQ ID NO: 280) |
| | | | (SEQ ID NO: 278) | | |
| heavy chain CDR3 | GGFWGSYRYWSAAFD | GGFWGSYRYWSAAFDI | GGFWGSYRYWSAAFD | ARGGFWGSYRYWSA | ARGGFWGSYRYWSAAF |
| | I (SEQ ID NO: 281) | (SEQ ID NO: 282) | I (SEQ ID NO: 283) | AFD (SEQ ID NO: 284) | DI (SEQ ID NO: 285) |
| light chain CDR1 | RSSQSLLHSNGYNYLD | RSSQSLLHSNGYNYLD | RSSQSLLHSNGYNYLD | LHSNGYNYLDWY | QSLLHSNGYNY |
| | (SEQ ID NO: 286) | (SEQ ID NO: 287) | (SEQ ID NO: 288) | (SEQ ID NO: 289) | (SEQ ID NO: 290) |
| light chain CDR2 | LGSNRAS | LGSNRAS | LGSNRAS | LLIYLGSNRA | LG |
| | (SEQ ID NO: 291) | (SEQ ID NO: 292) | (SEQ ID NO: 293) | (SEQ ID NO: 294) | (SEQ ID NO: 295) |
| light chain CDR3 | MQALQTPPT | MQALQTPPT | MQALQTPPT | MQALQTPP | MQALQTPPT |
| | (SEQ ID NO: 296) | (SEQ ID NO: 297) | (SEQ ID NO: 298) | (SEQ ID NO: 299) | (SEQ ID NO: 300) |

TABLE 11
Antibody YU1019-E05b

| | |
|---|---|
| VH | EVQLVESGAEVKKPGASVKVSCKASGYTFTSYGITWVRQAPGQGLEWMGWISAYNGNTNYAQKLQGRVTMTTDTSTSTAYMELRSLR |
| | SDDTAVYYCARHHPTGGSATIVFDYWGQGTLVTVSS |
| VL | DVVMTQSPLSLPVTPGEPASISCRSSQSLLHSNGYNYLDWYLQKPGQSPQLLIYLGSNRASGVPDRFRGSGSGTDFTLKISRVEAE |
| | DVGVYYCMQALQTPGFGQGTKVEIK |

TABLE 33
Antibody YU1019-E05b
CDRs

| | Chothia | AbM | Kabat | Contact | IMGT |
|---|---|---|---|---|---|
| heavy chain CDR1 | GYTFTSY | GYTFTSYGIT | SYGIT | TSYGIT | GYTFTSYG |
| | (SEQ ID NO: 301) | (SEQ ID NO: 302) | (SEQ ID NO: 303) | (SEQ ID NO: 304) | (SEQ ID NO: 305) |
| heavy chain CDR2 | SAYNGN | WISAYNGNTN | WISAYNGNTNYAQKL | WMGWISAYNGNTN | ISAYNGNT |
| | (SEQ ID NO: 306) | (SEQ ID NO: 307) | QG | (SEQ ID NO: 309) | (SEQ ID NO: 310) |
| | | | (SEQ ID NO: 308) | | |
| heavy chain CDR3 | HHPTGGSATIVFDY | HHPTGGSATIVFDY | HHPTGGSATIVFDY | ARHHPTGGSATIVFD | ARHHPTGGSATIVFDY |
| | (SEQ ID NO: 311) | (SEQ ID NO: 312) | (SEQ ID NO: 313) | (SEQ ID NO: 314) | (SEQ ID NO: 315) |
| light chain CDR1 | RSSQSLLHSNGYNYL | RSSQSLLHSNGYNYLD | RSSQSLLHSNGYNYLD | LHSNGYNYLDWY | QSLLHSNGYNY |
| | D (SEQ ID NO: 316) | (SEQ ID NO: 317) | (SEQ ID NO: 318) | (SEQ ID NO: 319) | (SEQ ID NO: 320) |
| light chain CDR2 | LGSNRAS | LGSNRAS | LGSNRAS | LLIYLGSNRA | LG |
| | (SEQ ID NO: 321) | (SEQ ID NO: 322) | (SEQ ID NO: 323) | (SEQ ID NO: 324) | (SEQ ID NO: 325) |
| light chain CDR3 | MQALQTPLT | MQALQTPLT (SEQ ID | MQALQTPLT (SEQ ID | MQALQTPL (SEQ ID | MQALQTPLT (SEQ ID |
| | (SEQ ID NO: 326) | NO: 327) | NO: 328) | NO: 329) | NO: 330) |

TABLE 12
Antibody YU1019-F10

| | |
|---|---|
| VH | QVQLVQSGAEVKKPGASVKVSCKASGYTFTSYYMHWVRQAPGQGLEWMGIINPSGGSTSYAQKFQGRVTMTRDTSTSTVYMELSSLR |
| | SEDTAVYYCARAPGKFPLDYWGQGTLVTVSS |
| VL | DVVMTQSPLSLPVTPGEPASISCRSSQSLLHSNGYNYLDWYLQKPGQSPQLLIYLGSNRASGVPDRFRGSGSGTDFTLKISRVEAE |
| | DVGVYYCMQALQTPGFGQGTKVEIK |

TABLE 34
Antibody YU1019-F10
CDRs

| | Chothia | AbM | Kabat | Contact | IMGT |
|---|---|---|---|---|---|
| heavy chain CDR1 | GYTFTSY | GYTFTSYYMH | SYYMH | TSYYMH | GYTFTSYY |
| | (SEQ ID NO: 331) | (SEQ ID NO: 332) | (SEQ ID NO: 333) | (SEQ ID NO: 334) | (SEQ ID NO: 335) |
| heavy chain CDR2 | NPSGGS | IINPSGGSTS | IINPSGGSTSYAQKFQG | WMGIINPSGGSTS | INPSGGST |
| | (SEQ ID NO: 336) | (SEQ ID NO: 337) | (SEQ ID NO: 338) | (SEQ ID NO: 339) | (SEQ ID NO: 340) |
| heavy chain CDR3 | APGKFPLDY | APGKFPLDY | APGKFPLDY | ARAPGKFPLD | ARAPGKFPLDY |
| | (SEQ ID NO: 341) | (SEQ ID NO: 342) | (SEQ ID NO: 343) | (SEQ ID NO: 344) | (SEQ ID NO: 345) |
| light chain CDR1 | RSSQSLLHSNGYNYL | RSSQSLLHSNGYNYLD | RSSQSLLHSNGYNYLD | LHSNGYNYLDWY | QSLLHSNGYNY |
| | D (SEQ ID NO: 346) | (SEQ ID NO: 347) | (SEQ ID NO: 348) | (SEQ ID NO: 349) | (SEQ ID NO: 350) |
| light chain CDR2 | LGSNRAS | LGSNRAS | LGSNRAS | LLIYLGSNRA | LG |
| | (SEQ ID NO: 351) | (SEQ ID NO: 352) | (SEQ ID NO: 353) | (SEQ ID NO: 354) | (SEQ ID NO: 355) |
| light chain CDR3 | MQALQTPG | MQALQTPG | MQALQTPG | MQALQTP | MQALQTPG |
| | (SEQ ID NO: 356) | (SEQ ID NO: 357) | (SEQ ID NO: 358) | (SEQ ID NO: 359) | (SEQ ID NO: 360) |

TABLE 13
Antibody YU1018-G10

| | |
|---|---|
| VH | EVQLVQSGAEVKKPGASVKVSCKASGYTFTSYYMHWVRQAPGQGLEWMGIINPSGGSTSYAQKFQGRVTMTRDTSTSTVYMELSS |
| | LRSEDTAVYYCARAPGKFPLDYWGQGTLVTVSS |
| VL | DVVMTQSPLSLPVTPGEPASISCRSSQSLLHSNGYNYLDWYLQKPGQSPQLLIYLGSNRASGVPDRFSGSGSGTDFTLKISRVE |
| | AEDVGVYYCMQALQTPWTFGQGTKLEIK |

TABLE 35
Antibody YU1018-G10
CDRs

| | Chothia | AbM | Kabat | Contact | IMGT |
|---|---|---|---|---|---|
| heavy chain | GYTFTSY | GYTFTSYYMH | SYYMH | TSYYMH | GYTFTSYY |
| CDR1 | (SEQ ID NO: 361) | (SEQ ID NO: 362) | (SEQ ID NO: 363) | (SEQ ID NO: 364) | (SEQ ID NO: 365) |
| heavy chain | NPSGGS | IINPSGGSTS | IINPSGGSTSYAQKFQG | WMGIINPSGGSTS | INPSGGST |
| CDR2 | (SEQ ID NO: 366) | (SEQ ID NO: 367) | (SEQ ID NO: 368) | (SEQ ID NO: 369) | (SEQ ID NO: 370) |
| heavy chain | APGKFPLDY | APGKFPLDY | APGKFPLDY | ARAPGKFPLD | ARAPGKFPLDY |
| CDR3 | (SEQ ID NO: 371) | (SEQ ID NO: 372) | (SEQ ID NO: 373) | (SEQ ID NO: 374) | (SEQ ID NO: 375) |
| light chain CDR1 | RSSQSLLHSNGYNYLD | RSSQSLLHSNGYNYLD | RSSQSLLHSNGYNYLD | LHSNGYNYLDWY | QSLLHSNGYNY |
| | (SEQ ID NO: 376) | (SEQ ID NO: 377) | (SEQ ID NO: 378) | (SEQ ID NO: 379) | (SEQ ID NO: 380) |
| light chain CDR2 | LGSNRAS | LGSNRAS | LGSNRAS | LLIYLGSNRA | LG |
| | (SEQ ID NO: 381) | (SEQ ID NO: 382) | (SEQ ID NO: 383) | (SEQ ID NO: 384) | (SEQ ID NO: 385) |
| light chain CDR3 | MQALQTPWT | MQALQTPWT | MQALQTPWT | MQALQTPW | MQALQTPWT |
| | (SEQ ID NO: 386) | (SEQ ID NO: 387) | (SEQ ID NO: 388) | (SEQ ID NO: 389) | (SEQ ID NO: 390) |

TABLE 14
Antibody YU1018-E04

| | |
|---|---|
| VH | QVQLVQSGAEVKKPGESLKISCKGSGYSFTSYWIGWVRQMPGKGLEWMGIIYPGDSDTRYSPSFQGQVTISADKSISTAYLQWSS |
| | LKASDTAMYYCARAIVGATSGYWFDPWGQGTLVTVSS |
| VL | DIVMTQSPLSLPVTPGEPASISCRSSQSLLHSNGYNYLDWYLQKPGQSPQLLIYLGSNRASGVPDRFSGSGSGTDFTLKISRVE |
| | AEDVGVYYCMQALQTPLTFGGGTKVEIK |

TABLE 36
Antibody YU1018-E04

| CDRs | Chothia | AbM | Kabat | Contact | IMGT |
|---|---|---|---|---|---|
| heavy chain CDR1 | GYSFTSY | GYSFTSYWIG | SYWIG | TSYWIG | GYSFTSYW |
| | (SEQ ID NO: 391) | (SEQ ID NO: 392) | (SEQ ID NO: 393) | (SEQ ID NO: 394) | (SEQ ID NO: 395) |
| heavy chain CDR2 | YPGDSD | IYPGDSDTR | IYPGDSDTRYSPSFQG | WMGIIYPGDSDTR | IYPGDSDT |
| | (SEQ ID NO: 396) | (SEQ ID NO: 397) | (SEQ ID NO: 398) | (SEQ ID NO: 399) | (SEQ ID NO: 400) |
| heavy chain CDR3 | AIVGATSGYWFDP | AIVGATSGYWFDP | AIVGATSGYWFDP | ARAIVGATSGYWFD | ARAIVGATSGYWFDP |
| | (SEQ ID NO: 401) | (SEQ ID NO: 402) | (SEQ ID NO: 403) | (SEQ ID NO: 404) | (SEQ ID NO: 405) |
| light chain CDR1 | RSSQSLLHSNGYNYL | RSSQSLLHSNGYNYLD | RSSQSLLHSNGYNYLD | LHSNGYNYLDWY | QSLLHSNGYNY |
| | D (SEQ ID NO: 406) | (SEQ ID NO: 407) | (SEQ ID NO: 408) | (SEQ ID NO: 409) | (SEQ ID NO: 410) |
| light chain CDR2 | LGSNRAS | LGSNRAS | LGSNRAS | LLIYLGSNRA | LG |
| | (SEQ ID NO: 411) | (SEQ ID NO: 412) | (SEQ ID NO: 413) | (SEQ ID NO: 414) | (SEQ ID NO: 415) |
| light chain CDR3 | MQALQTPLT | MQALQTPLT | MQALQTPLT | MQALQTPL | MQALQTPLT |
| | (SEQ ID NO: 416) | (SEQ ID NO: 417) | (SEQ ID NO: 418) | (SEQ ID NO: 419) | (SEQ ID NO: 420) |

TABLE 15
Antibody YU1018-E07

| | |
|---|---|
| VH | EVQLLESGGGVVQPGRSLRLSCAASGFTFSSYAMHWVRQAPGKGLEWVAVISYDGSNKYYADSVKGRFTISRDNSKNTLYLQMNS |
| | LRAEDTAVYYCASPVLRYFDWLPNYWGQGTTVTVSS |
| VL | DVVMTQSPLSLPVTLGQPASISCRSSQSLVYSDGNTYLNWFQQRPGQSPRRLIYKVSNRDSGVPDRFSGSGSGTDFTLKISRVEA |
| | EDVGVYYCMQGTHWPYSFGQGTKVEIK |

TABLE 37
Antibody YU1018-E07
CDRs

| | Chothia | AbM | Kabat | Contact | IMGT |
|---|---|---|---|---|---|
| heavy | GFTFSSY | GFTFSSYAMH | SYAMH | SSYAMH | GFTFSSYA |
| chain | (SEQ ID NO: 421) | (SEQ ID NO: 422) | (SEQ ID NO: 423) | (SEQ ID NO: 424) | (SEQ ID NO: 425) |
| CDR1 | | | | | |
| heavy | SYDGSN | VISYDGSNKY | VISYDGSNKYYADSVK | WVAVISYDGSNKY | ISYDGSNK |
| chain | (SEQ ID NO: 426) | (SEQ ID NO: 427) | G (SEQ ID NO: 428) | (SEQ ID NO: 429) | (SEQ ID NO: 430) |
| CDR2 | | | | | |
| heavy | PVLRYFDWLPNY | PVLRYFDWLPNY | PVLRYFDWLPNY | ASPVLRYFDWLPN | ASPVLRYFDWLPNY |
| chain | (SEQ ID NO: 431) | (SEQ ID NO: 432) | (SEQ ID NO: 433) | (SEQ ID NO: 434) | (SEQ ID NO: 435) |
| CDR3 | | | | | |
| light | RSSQSLVYSDGNTYL | RSSQSLVYSDGNTYLN | RSSQSLVYSDGNTYLN | VYSDGNTYLNWF | QSLVYSDGNTY |
| chain | N | (SEQ ID NO: 437) | (SEQ ID NO: 438) | (SEQ ID NO: 439) | (SEQ ID NO: 440) |
| CDR1 | (SEQ ID NO: 436) | | | | |
| light | KVSNRDS | KVSNRDS | KVSNRDS | RLIYKVSNRD | KV |
| chain | (SEQ ID NO: 441) | (SEQ ID NO: 442) | (SEQ ID NO: 443) | (SEQ ID NO: 444) | (SEQ ID NO: 445) |
| CDR2 | | | | | |
| light | MQGTHWPYS | MQGTHWPYS | MQGTHWPYS | MQGTHWPY | MQGTHWPYS |
| chain | (SEQ ID NO: 446) | (SEQ ID NO: 447) | (SEQ ID NO: 448) | (SEQ ID NO: 449) | (SEQ ID NO: 450) |
| CDR3 | | | | | |

TABLE 16
Antibody YU1018-E12

| | |
|---|---|
| VH | QVQLVQSGAEVKKPGESLKISCKGSGYSFTSYWIGWVRQMPGKGLEWMGIIYPGDSDTRYSPSFQGQVTISADKSISTAYLQWSSLK |
| | ASDTAMYYCARGHGATAFDIWGQGTMVTVSS |
| VL | DVVMTQSPLSLPVTPGEPASISCRSSQSLLHSNGYNYLDWYLQKPGQSPQLLIYLGSNRASGVPDRFSGSGSGTDFTLKISRVEAED |
| | VGVYYCMQALQTPPTFGPGTKLEIK |

TABLE 38
Antibody YU1018-E12
CDRs

| | Chothia | AbM | Kabat | Contact | IMGT |
|---|---|---|---|---|---|
| heavy | GYSFTSY | GYSFTSYWIG | SYWIG | TSYWIG | GYSFTSYW |
| chain | (SEQ ID NO: 451) | (SEQ ID NO: 452) | (SEQ ID NO: 453) | (SEQ ID NO: 454) | (SEQ ID NO: 455) |
| CDR1 | | | | | |
| heavy | YPGDSD | IIYPGDSDTR | IIYPGDSDTRYSPSFQG | WMGIIYPGDSDTR | IYPGDSDT |
| chain | (SEQ ID NO: 456) | (SEQ ID NO: 457) | (SEQ ID NO: 458) | (SEQ ID NO: 459) | (SEQ ID NO: 460) |
| CDR2 | | | | | |
| heavy | GHGATAFDI | GHGATAFDI | GHGATAFDI | ARGHGATAFD | ARGHGATAFDI |
| chain | (SEQ ID NO: 461) | (SEQ ID NO: 462) | (SEQ ID NO: 463) | (SEQ ID NO: 464) | (SEQ ID NO: 465) |
| CDR3 | | | | | |
| light | RSSQSLLHSNGYNYL | RSSQSLLHSNGYNYLD | RSSQSLLHSNGYNYLD | LHSNGYNYLDWY | QSLLHSNGYNY |
| chain | D | (SEQ ID NO: 467) | (SEQ ID NO: 468) | (SEQ ID NO: 469) | (SEQ ID NO: 470) |
| CDR1 | (SEQ ID NO: 466) | | | | |
| light | LGSNRAS | LGSNRAS | LGSNRAS | LLIYLGSNRA | LG |
| chain | (SEQ ID NO: 471) | (SEQ ID NO: 472) | (SEQ ID NO: 473) | (SEQ ID NO: 474) | (SEQ ID NO: 475) |
| CDR2 | | | | | |
| light | MQALQTPPT | MQALQTPPT | MQALQTPPT | MQALQTPP | MQALQTPPT |
| chain | (SEQ ID NO: 476) | (SEQ ID NO: 477) | (SEQ ID NO: 478) | (SEQ ID NO: 479) | (SEQ ID NO: 480) |
| CDR3 | | | | | |

TABLE 17
Antibody YU1018-D08

| | |
|---|---|
| VH | QVQLVQSGAEVKKPGSSVKVSCKASGGTFSSYAISWVRQAPGQGLEWMGGIIPIFGTANYAQKFQGRVTITADKSTSTAYMELSSLR |
| | SEDTAVYYCARDRYYDSSGYYLMDPWGQGTLVTVSS |
| VL | DIVMTQSPLSSPVTLGQPASISCRSSQSLVHSDGNTYLSWLQQRPGQPPRLLIYKISNRFSGVPDRFSGSGAGTDFTLKISRVEAED |
| | VGVYYCMQATQFPLTFGGGTRLEIK |

TABLE 39
Antibody YU1018-D08
CDRs

| | Chothia | AbM | Kabat | Contact | IMGT |
|---|---|---|---|---|---|
| heavy | GGTFSSY | GGTFSSYAIS | SYAIS | SSYAIS | GGTESSYA |
| chain | (SEQ ID NO: 481) | (SEQ ID NO: 482) | (SEQ ID NO: 483) | (SEQ ID NO: 484) | (SEQ ID NO: 485) |
| CDR1 | | | | | |
| heavy | IPIFGT | GIIPIFGTAN | GIIPIFGTANYAQKFQG | WMGGIIPIFGTAN | IIPIFGTA |
| chain | (SEQ ID NO: 486) | (SEQ ID NO: 487) | (SEQ ID NO: 488) | (SEQ ID NO: 489) | (SEQ ID NO: 490) |
| CDR2 | | | | | |
| heavy | DRYYDSSGYYLMDP | DRYYDSSGYYLMDP | DRYYDSSGYYLMDP | ARDRYYDSSGYYLM | ARDRYYDSSGYYLMDP |
| chain | (SEQ ID NO: 491) | (SEQ ID NO: 492) | (SEQ ID NO: 493) | D | (SEQ ID NO: 495) |
| CDR3 | | | | (SEQ ID NO: 494) | |
| light | RSSQSLVHSDGNTYL | RSSQSLVHSDGNTYLS | RSSQSLVHSDGNTYLS | VHSDGNTYLSWL | QSLVHSDGNTY |
| chain | S | (SEQ ID NO: 497) | (SEQ ID NO: 498) | (SEQ ID NO: 499) | (SEQ ID NO: 500) |
| CDR1 | (SEQ ID NO: 496) | | | | |
| light | KISNRFS | KISNRFS | KISNRFS | LLIYKISNRE | KI |
| chain | (SEQ ID NO: 501) | (SEQ ID NO: 502) | (SEQ ID NO: 503) | (SEQ ID NO: 504) | (SEQ ID NO: 505) |
| CDR2 | | | | | |
| light | MQATQFPLT | MQATQFPLT | MQATQFPLT | MQATQFPL | MQATQFPLT |
| chain | (SEQ ID NO: 506) | (SEQ ID NO: 507) | (SEQ ID NO: 508) | (SEQ ID NO: 509) | (SEQ ID NO: 510) |
| CDR3 | | | | | |

TABLE 18
Antibody YU1018-E10

| | |
|---|---|
| VH | QVQLVQSGAEVKKPGASVKVSCKASGYTFTSYYMHWVRQAPGQGLEWMGIINPSGGSTSYAQKFQGRVTMTRDTSTSTVYMELSS |
| | LRSEDTAVYYCARAPGKFPLDYWGQGTLVTVSS |
| VL | DVVMTQSPLSLPVTPGEPASISCRSSQSLLKSNGYNYLDWYLQKPGQSPQLLIYLGSNRASGVPDRFSGSGSGTDFSLKIRRVEA |
| | EDVGVYYCMQTLQTPYTFGQGTKLEIK |

TABLE 40
Antibody YU1018-E10
CDRs

| | Chothia | AbM | Kabat | Contact | IMGT |
|---|---|---|---|---|---|
| heavy | GYTFTSY | GYTFTSYYMH | SYYMH | TSYYMH | GYTFTSYY |
| chain | (SEQ ID NO: 511) | (SEQ ID NO: 512) | (SEQ ID NO: 513) | (SEQ ID NO: 514) | (SEQ ID NO: 515) |
| CDR1 | | | | | |
| heavy | NPSGGS | IINPSGGSTS | IINPSGGSTSYAQKFQG | WMGIINPSGGSTS | INPSGGST |
| chain | (SEQ ID NO: 516) | (SEQ ID NO: 517) | (SEQ ID NO: 518) | (SEQ ID NO: 519) | (SEQ ID NO: 520) |
| CDR2 | | | | | |
| heavy | APGKFPLDY | APGKFPLDY | APGKFPLDY | ARAPGKFPLD | ARAPGKFPLDY |
| chain | (SEQ ID NO: 521) | (SEQ ID NO: 522) | (SEQ ID NO: 523) | (SEQ ID NO: 524) | (SEQ ID NO: 525) |
| CDR3 | | | | | |
| light | RSSQSLLKSNGYNYL | RSSQSLLKSNGYNYLD | RSSQSLLKSNGYNYLD | LKSNGYNYLDWY | QSLIKSNGYNY |
| chain | D | (SEQ ID NO: 527) | (SEQ ID NO: 528) | (SEQ ID NO: 529) | (SEQ ID NO: 530) |
| CDR1 | (SEQ ID NO: 526) | | | | |
| light | LGSNRAS | LGSNRAS | LGSNRAS | LLIYLGSNRA | LG |
| chain | (SEQ ID NO: 531) | (SEQ ID NO: 532) | (SEQ ID NO: 533) | (SEQ ID NO: 534) | (SEQ ID NO: 535) |
| CDR2 | | | | | |
| light | MQTLQTPYT | MQTLQTPYT | MQTLQTPYT | MOTLQTPY | MQTLQTPYT |
| chain | (SEQ ID NO: 536) | (SEQ ID NO: 537) | (SEQ ID NO: 538) | (SEQ ID NO: 539) | (SEQ ID NO: 540) |
| CDR3 | | | | | |

TABLE 19
Antibody YU1018-D06

| | |
|---|---|
| VH | QVQLVESGPGLVKPSQTLSLTCTVSGGSISSGGYYWSWIRQHPGKGLEWIGYIYYSGSTYYNPSLKSRVTISVDTSKNQFSLKLSS |
| | VTAADTAVYYCARSGAAGIPWFDPWGQGTLVTVSS |
| VL | QSVLTQPPSVSAAPGQKVTISCSGSSSNIGNNYVSWYQQLPGTAPKLLIYENNERPSGIPDRLSGSKSGTSATLAITGLQTGDEAD |
| | YYCGTWDSSLSAVVFGGGTKLTVL |

TABLE 41
Antibody YU1018-D06
CDRs

| | Chothia | AbM | Kabat | Contact | IMGT |
|---|---|---|---|---|---|
| heavy | GGSISSGGY | GGSISSGGYYWS | SGGYYWS | SSGGYYWS | GGSISSGGYY |
| chain | (SEQ ID NO: 541) | (SEQ ID NO: 542) | (SEQ ID NO: 543) | (SEQ ID NO: 544) | (SEQ ID NO: 545) |
| CDR1 | | | | | |
| heavy | YYSGS | YIYYSGSTY | YIYYSGSTYYNPSLKS | WIGYIYYSGSTY | IYYSGST |
| chain | (SEQ ID NO: 546) | (SEQ ID NO: 547) | (SEQ ID NO: 548) | (SEQ ID NO: 549) | (SEQ ID NO: 550) |
| CDR2 | | | | | |
| heavy | SGAAGIPWFDP | SGAAGIPWEDP | SGAAGIPWFDP | ARSGAAGIPWED | ARSGAAGIPWFDP |
| chain | (SEQ ID NO: 551) | (SEQ ID NO: 552) | (SEQ ID NO: 553) | (SEQ ID NO: 554) | (SEQ ID NO: 555) |
| CDR3 | | | | | |
| light | SGSSSNIGNNYVS | SGSSSNIGNNYVS | SGSSSNIGNNYVS | IGNNYVSWY | SSNIGNNY |
| chain | (SEQ ID NO: 556) | (SEQ ID NO: 557) | (SEQ ID NO: 558) | (SEQ ID NO: 559) | (SEQ ID NO: 560) |
| CDR1 | | | | | |
| light | ENNERPS | ENNERPS | ENNERPS | LLIYENNERP | EN |
| chain | (SEQ ID NO: 561) | (SEQ ID NO: 562) | (SEQ ID NO: 563) | (SEQ ID NO: 564) | (SEQ ID NO: 565) |
| CDR2 | | | | | |
| light | GTWDSSLSAVV | GTWDSSLSAVV | GTWDSSLSAVV | GTWDSSLSAV | GTWDSSLSAVV |
| chain | (SEQ ID NO: 566) | (SEQ ID NO: 567) | (SEQ ID NO: 568) | (SEQ ID NO: 569) | (SEQ ID NO: 570) |
| CDR3 | | | | | |

TABLE 20
Antibody YU1018-E01

| | |
|---|---|
| VH | QVQLVQSGAEVKKPGASVKVSCKASGYTFTSYGISWVRQAPGRGLEWMGWISAYNGNTNYAQKLQGRVTMTTDTSTSTAYMELRS |
| | LRSDDTAVYYCARGPRDGYNDYWGQGTLVTVSS |
| VL | DVVMTQSPLSLPVTPGEPASISCRSSQSLLHSNGYNYLNWYLQKPGQSPQLLIYLGSNRASGVPDRFSGSGSGTDFTLKISRVEA |
| | EDVGVYYCMQPLQTPLTFGGGTKLEIK |

TABLE 42
Antibody YU1018-E01
CDRs

| | Chothia | AbM | Kabat | Contact | IMGT |
|---|---|---|---|---|---|
| heavy | GYTFTSY | GYTFTSYGIS | SYGIS | TSYGIS | GYTFTSYG |
| chain | (SEQ ID NO: 571) | (SEQ ID NO: 572) | (SEQ ID NO: 573) | (SEQ ID NO: 574) | (SEQ ID NO: 575) |
| CDR1 | | | | | |
| heavy | SAYNGN | WISAYNGNTN | WISAYNGNTNYAQKL | WMGWISAYNGNTN | ISAYNGNT |
| chain | (SEQ ID NO: 576) | (SEQ ID NO: 577) | QG | (SEQ ID NO: 579) | (SEQ ID NO: 580) |
| CDR2 | | | (SEQ ID NO: 578) | | |
| heavy | GPRDGYNDY | GPRDGYNDY | GPRDGYNDY | ARGPRDGYND | ARGPRDGYNDY |
| chain | (SEQ ID NO: 581) | (SEQ ID NO: 582) | (SEQ ID NO: 583) | (SEQ ID NO: 584) | (SEQ ID NO: 585) |
| CDR3 | | | | | |
| light | RSSQSLLHSNGYNYL | RSSQSLLHSNGYNYLN | RSSQSLLHSNGYNYLN | LHSNGYNYLNWY | QSLLHSNGYNY |
| chain | N | (SEQ ID NO: 587) | (SEQ ID NO: 588) | (SEQ ID NO: 589) | (SEQ ID NO: 590) |
| CDR1 | (SEQ ID NO: 586) | | | | |
| light | LGSNRAS | LGSNRAS | LGSNRAS | LLIYLGSNRA | LG |
| chain | (SEQ ID NO: 591) | (SEQ ID NO: 592) | (SEQ ID NO: 593) | (SEQ ID NO: 594) | (SEQ ID NO: 595) |
| CDR2 | | | | | |
| light | MQPLQTPLT | MQPLQTPLT | MQPLQTPLT | MQPLQTPL | MQPLQTPLI |
| chain | (SEQ ID NO: 596) | (SEQ ID NO: 597) | (SEQ ID NO: 598) | (SEQ ID NO: 599) | (SEQ ID NO: 600) |
| CDR3 | | | | | |

TABLE 21
Antibody YU1018-A04

| | |
|---|---|
| VH | QVQLVQSGAEVKKPGASVKVSCKASGYTFTSYGISWVRQAPGQGLEWMGWISAYNGNTNYAQKLQGRVTMTTDTSTSTAYMELRS |
| | LRSDDTAVYYCARMAGWELIDPWGQGTLVTVSS |
| VL | DVVMTQSPLSLPVTPGEPASISCRSSQSLLHSNGYNYLDWYLQKPGKSPQLLIYLGSNRASGVPDRFSGSGSGTDFTLKISRVEA |
| | EDVGVYYCMQALQTPYSFGQGTKVEIK |

TABLE 43
Antibody
YU1018-A04
CDRs

| | Chothia | AbM | Kabat | Contact | IMGT |
|---|---|---|---|---|---|
| heavy | GYTFTSY | GYTFTSYGIS | SYGIS | TSYGIS | GYTFTSYG |
| chain | (SEQ ID NO: 601) | (SEQ ID NO: 602) | (SEQ ID NO: 603) | (SEQ ID NO: 604) | (SEQ ID NO: 605) |
| CDR1 | | | | | |
| heavy | SAYNGN | WISAYNGNIN | WISAYNGNTNYAQKL | WMGWISAYNGNTN | ISAYNGNT |
| chain | (SEQ ID NO: 606) | (SEQ ID NO: 607) | QG | (SEQ ID NO: 609) | (SEQ ID NO: 610) |
| CDR2 | | | (SEQ ID NO: 608) | | |
| heavy | MAGWELIDP | MAGWELIDP | MAGWELIDP | ARMAGWELID | ARMAGWELIDP |
| chain | (SEQ ID NO: 611) | (SEQ ID NO: 612) | (SEQ ID NO: 613) | (SEQ ID NO: 614) | (SEQ ID NO: 615) |
| CDR3 | | | | | |
| light | RSSQSLLHSNGYNYL | RSSQSLLHSNGYNYLD | RSSQSLLHSNGYNYLD | LHSNGYNYLDWY | QSLLHSNGYNY |
| chain | D | (SEQ ID NO: 617) | (SEQ ID NO: 618) | (SEQ ID NO: 619) | (SEQ ID NO: 620) |
| CDR1 | (SEQ ID NO: 616) | | | | |
| light | LGSNRAS | LGSNRAS | LGSNRAS | LLIYLGSNRA | LG |
| chain | (SEQ ID NO: 621) | (SEQ ID NO: 622) | (SEQ ID NO: 623) | (SEQ ID NO: 624) | (SEQ ID NO: 625) |
| CDR2 | | | | | |
| light | MQALQTPYS | MQALQTPYS | MQALQTPYS | MQALQTPY | MQALQTPYS |
| chain | (SEQ ID NO: 626) | (SEQ ID NO: 627) | (SEQ ID NO: 628) | (SEQ ID NO: 629) | (SEQ ID NO: 630) |
| CDR3 | | | | | |

TABLE 22
Antibody YU1018-G09

| | |
|---|---|
| VH | EVQLLESGAEVKKPGESLKISCKGSGYSFTSYWIGWVRQMPGKGLEWMGIIYPGDSDTRYSPSFQGQVTISADKSISTAYLQWSSL |
| | KASDTAMYYCARGVGSVVFDYWGQGTLVTVSS |
| VL | DVVMTQSPLPLPVTPGEPASISCRSSQSLLHSNGYNYLDWYLQKPGQSPQLLIYLGSNRASGVPDRFSGSGSGTDFTLKISRVEAE |
| | DVGVYYCMQALQTPPTFGQGTKVEIK |

TABLE 44
Antibody
YU1018-G09
CDRs

| | Chothia | AbM | Kabat | Contact | IMGT |
|---|---|---|---|---|---|
| heavy | GYSFTSY | GYSFTSYWIG | SYWIG | TSYWIG | GYSFTSYW |
| chain | (SEQ ID NO: 631) | (SEQ ID NO: 632) | (SEQ ID NO: 633) | (SEQ ID NO: 634) | (SEQ ID NO: 635) |
| CDR1 | | | | | |
| heavy | YPGDSD | IIYPGDSDTR | IYPGDSDTRYSPSFQG | WMGIIYPGDSDTR | IYPGDSDT |
| chain | (SEQ ID NO: 636) | (SEQ ID NO: 637) | (SEQ ID NO: 638) | (SEQ ID NO: 639) | (SEQ ID NO: 640) |
| CDR2 | | | | | |
| heavy | CVGSVVEDY | GVGSVVEDY | GVGSVVEDY | ARGVGSVVED | ARGVGSVVFD |
| chain | (SEQ ID NO: 641) | (SEQ ID NO: 642) | (SEQ ID NO: 643) | (SEQ ID NO: 644) | (SEQ ID NO: 645) |
| CDR3 | | | | | |
| light | RSSQSLLHSNGYNYL | RSSQSLLHSNGYNYLD | RSSQSLLHSNGYNYLD | LHSNGYNYLDWY | QSLLHSNGYNY |
| chain | D | (SEQ ID NO: 647) | (SEQ ID NO: 648) | (SEQ ID NO: 649) | (SEQ ID NO: 650) |
| CDR1 | (SEQ ID NO: 646) | | | | |
| light | LGSNRAS | LGSNRAS | LGSNRAS | LLIYLGSNRA | LG |
| chain | (SEQ ID NO: 651) | (SEQ ID NO: 652) | (SEQ ID NO: 653) | (SEQ ID NO: 654) | (SEQ ID NO: 655) |
| CDR2 | | | | | |
| light | MQALQTPPT | MQALQTPPT | MQALQTPPT | MQALQTPP | MQALQTPPT |
| chain | (SEQ ID NO: 656) | (SEQ ID NO: 657) | (SEQ ID NO: 658) | (SEQ ID NO: 659) | (SEQ ID NO: 660) |
| CDR3 | | | | | |

In some embodiments, the antibody or antigen-binding fragment comprises three heavy chain CDRs and three light chain CDRs having sequences selected from:
a) SEQ ID NOs: 3, 8, and 13 and SEQ ID NOs: 18, 23, and 28 (antibody E05a);
b) SEQ ID NOs: 33, 38, and 43 and SEQ ID NOs: 48, 53, and 58 (antibody H08);
c) SED ID NOs: 63, 68, and 73 and SED ID NOs: 78, 83, and 88 (antibody F06);
d) SEQ ID NOs: 93, 98, and 103 and SEQ ID NOs: 108, 113, and 118 (antibody A12);
e) SED ID NOs: 123, 128, and 133 and SED ID NOs: 138, 143, and 148 (antibody G01);
f) SED ID NOs: 153, 158, and 163 and SED ID NOs: 168, 173 and 178 (antibody E11);
g) SEQ ID NOs: 183, 188, and 193 and SEQ ID NOs: 198, 203, and 208 (antibody B06);
h) SED ID NOs: 213, 218, and 223 and SED ID NOs: 228, 233, and 238 (antibody D12);
i) SED ID NOs: 243, 248, and 253 and SED ID NOs: 258, 263 and 268 (antibody H01);
j) SED ID NOs: 273, 278, and 283 and SED ID NOs: 288, 293, and 298 (antibody C11);
k) SED ID NOs: 303, 308, and 313 and SED ID NOs: 318, 323, and 328 (antibody E05b);
l) SED ID NOs: 333, 338, and 343 and SED ID NOs: 348, 353 and 358 (antibody F10);
m) SED ID NOs: 363, 368 and 373 and SED ID NOs: 378, 383 and 388 (antibody G10);
n) SEQ ID NOs: 393, 398, and 403 and SEQ ID NOs: 408, 413, and 418 (antibody E04);
o) SED ID NOs: 423, 428, and 433 and SED ID NOs: 438, 443, and 448 (antibody E07);
p) SEQ ID NOs: 453, 458, and 463 and SED ID NOs: 468, 473, and 478 (antibody E12);
q) SED ID NOs: 483, 488, and 493 and SED ID NOs: 498, and 503, and 508 (antibody D08);
r) SED ID NOs: 513, 518, and 523 and SED ID NOs: 528, 533, and 538 (antibody E10);
s) SEQ ID NOs: 543, 548, and 553 and SEQ ID NOs: 558, 563, and 568 (antibody D06); and
t) SED ID NOs: 573, 578, and 583 and SED ID NOs: 588, 593, and 598 (antibody E01),
or having sequences that differ from the selected CDR sequences (a)-(t) by at most two conservative amino acid substitutions in each CDR.

In some embodiments, the antibody or antigen-binding fragment comprises three heavy chain CDRs and three light chain CDRs with sequences identical to the selected CDRs (a)-(t). In some embodiments, the antibody or antigen-binding fragment comprises three heavy chain CDRs and three light chain CDRs having sequences that differ from the selected CDR sequences (a)-(t) by at most two conservative amino acid substitutions in each CDR, or by at most 1 conservative amino acid substitution in each CDR. In some embodiments, the antibody or antigen-binding fragment comprises three heavy chain CDRs and three light chain CDRs having sequences that differ from the selected CDR sequences (a)-(t) by 6 conservative amino acid changes in total across all 6 CDRs, 5 conservative amino acid substitutions in total across all 6 CDRs, 4 conservative amino acid substitutions in total across all 6 CDRs, 3 conservative amino acid substitutions in total across all 6 CDRs, 2 conservative amino acid substitutions in total across all 6 CDRs, or 1 conservative amino acid substitution total across all 6 CDRs.

In some embodiments, the antibody or antigen-binding fragment comprises three heavy chain CDRs and three light chain CDRs having sequences selected from:
b) SEQ ID NOs: 33, 38, and 43 and SEQ ID NOs: 48, 53 and 58 (antibody H08);
d) SEQ ID NOs: 93, 98, and 103 and SEQ ID NOs: 108, 113, and 118 (antibody A12);
g) SEQ ID NOs: 183, 188, and 193 and SEQ ID NOs: 198, 203, and 208 (antibody B06);
n) SEQ ID NOs: 393, 398, and 403 and SEQ ID NOs: 408, 413, and 418 (antibody E04);
s) SEQ ID NOs: 543, 548, and 553 and SEQ ID NOs: 558, 563, and 568 (antibody D06); and
t) SEQ ID NOs: 573, 578, and 583 and SEQ ID NOs: 588, 593, and 598 (antibody E01),
or having sequences that differ from the selected CDR sequences b), d), g), n) s), and t) by at most two conservative amino acid substitutions in each CDR.

In some embodiments, the antibody or antigen-binding fragment comprises three heavy chain CDRs and three light chain CDRs with sequences identical to the selected CDRs (b), (d), (g), (n), (s) or (t). In some embodiments, the antibody or antigen-binding fragment comprises three heavy chain CDRs and three light chain CDRs having sequences that differ from the selected CDR sequences (b), (d), (g), (n), (s) or (t) by at most two conservative amino acid substitutions in each CDR, or by at most 1 conservative amino acid substitution in each CDR. In some embodiments, the antibody or antigen-binding fragment comprises three heavy chain CDRs and three light chain CDRs having sequences that differ from the selected CDR sequences (b), (d), (g), (n), (s) or (t) by 6 conservative amino acid changes in total across all 6 CDRs, 5 conservative amino acid substitutions in total across all 6 CDRs, 4 conservative amino acid substitutions in total across all 6 CDRs, 3 conservative amino acid substitutions in total across all 6 CDRs, 2 conservative amino acid substitutions in total across all 6 CDRs, or 1 conservative amino acid substitution total across all 6 CDRs.

In some embodiments, the antibody framework regions are human antibody framework regions. In some embodiments, the antibody is a full length bivalent monospecific monoclonal antibody. In some embodiments, the antibody comprises human IgG1, IgG2, or IgG4 heavy chain constant regions. In some embodiments, the antibody comprises a human IgG1 constant region. In some embodiments, the antibody Fc region has at least one engineered mutation that reduces antibody binding to at least an Fc γ receptor. In some embodiments, the mutation is N297A. In some embodiments, the antibody Fc region has at least one engineered mutation that reduces complement fixation. In some embodiments, the mutation is K322A.

In some embodiments, the antibody is a Fab, optionally wherein the Fab is PEGylated.

In some embodiments, the antibody or antigen binding fragment is further capable of binding to cynomolgus monkey NBL1. In some embodiments, the antibody or antigen binding fragment is further capable of binding to mouse NBL1.

In some embodiments, the antibody is an IgG monoclonal antibody. In particular embodiments, the antibody is an IgG1 or IgG4 monoclonal antibody.

In some embodiments, the antibody is a human monoclonal antibody. In some embodiments, the antibody is a humanized antibody. In some embodiments, the antibody is a chimeric mouse-human antibody. In some embodiments, the agent comprises an NBL1-binding antigen binding fragment selected from a Fab, Fab′, F(ab′)₂, Fv, scFv, Fd, or diabody.

In some embodiments the antibody or antigen binding fragment has a binding affinity (K_{D}) for human NBL1 of less than 100 nM. In some embodiments the antibody or antigen binding fragment has a binding affinity (K_{D}) for human NBL1 of less than 10 nM. In some embodiments the antibody or antigen binding fragment has a binding affinity (K_{D}) for human NBL1 of less than 5 nM. In some embodiments the antibody or antigen binding fragment has a binding affinity (K_{D}) for human NBL1 of less than 1 nM.

In certain embodiments, the NBL1 dimerization inhibitor is an antibody. In some of these embodiments, the antibody is a monoclonal antibody. In some of these embodiments, the antibody is polyclonal. In some embodiments the dimerization inhibitor binds to at least one of the synonymous β-strands from each NBL1 monomer.

### 5.3.3. NBL1 Expression Inhibitors

In some embodiments, the agent is capable of inhibiting NBL1 expression.

In certain embodiments, the agent inhibits transcription of the NBL1 gene. In certain embodiments, the agent causes degradation of the NBL1 mRNA. In certain embodiments, the agent inhibits translation of the NBL1 mRNA. In certain embodiments, the agent targets NBL1 protein for degradation.

In specific embodiments, the agent is an antisense oligonucleotide. In specific embodiments, the agent mediates RNA interference. In particular RNA interference embodiments, the agent is a short hairpin RNA (shRNA) or short interfering RNA (siRNA). In specific embodiments, the agent is a microRNA (miRNA). In specific embodiments, the agent is a sequence-specific mRNA interferase.

### 5.4. Methods of Slowing Decline in Kidney Function

In a further aspect, methods are provided for slowing decline in kidney function in a subject who has, or is at risk for, type 1 or type 2 diabetes, or who has or is at risk for developing a glomerular disease. The method comprises administering to the subject an effective amount of an agent capable of inhibiting NBL1 activity. In various embodiments, the agent capable of inhibiting NBL1 activity is an inhibitor as described in Section 5.3.1, 5.3.2, or 5.3.3 above.

In some embodiments, the method slows progression of microalbuminuria, slows progression of macroalbuminuria, slows progression of proteinuria, or slows reduction of glomerular filtration rate (GFR).

In some embodiments, the method prevents onset of or slows progressive kidney function decline (PKFD). In some embodiments, the agent inhibits the progression of at least one or more symptoms associated with PKFD. In some embodiments, the rate of decline in kidney function of the subject in relation to an untreated control group is reduced by 10%, 20%, 30%, 40%, 50%, 60%, 70%, 80% or 90%.

In some embodiments, the method prevents onset of or slows progression to end-stage kidney disease (ESKD). In some embodiments, the method inhibits the progression of at least one or more symptoms associated with ESKD. In some embodiments, the method inhibits progression to required dialysis.

In some embodiments, the method slows progression of chronic kidney disease (CKD) stage 1 to stage 2, stage 2 to stage 3, stage 3A to stage 3B, stage 3B to stage 4, stage 4 to stage 5, or progression from stage 5 without dialysis to stage 5 with dialysis.

In some embodiments, the subject has type 1 diabetes. In some embodiments, the subject has type 2 diabetes. In some embodiments, the subject is pre-diabetic. In some embodiments, the subject is not prediabetic as measured by hemoglobin A1c levels or blood glucose levels but is at risk for type 1 diabetes or type 2 diabetes and has elevated NBL1 plasma levels. In some embodiments, the subject has a glomerular disorder. In particular embodiments, the subject has a glomerular disorder and does not have type 1 or type 2 diabetes. In particular embodiments, the glomerular disorder is selected from the group consisting of focal segmental glomerulosclerosis (FSGS), chronic glomerulopathies, hereditary nephritis, and minimal change disease.

In some embodiments, the method effectively reduces the availability of circulating free NBL1 in subjects having type 1 or type 2 diabetes, or who are prediabetic, or who are at risk for type 1 diabetes or type 2 diabetes and have elevated NBL1 plasma levels, as compared to normal subjects who do not have type 1 or type 2 diabetes or prediabetes. In some embodiments, the method effectively reduces the availability of circulating free NBL1 in subjects who have a glomerular disease. In certain embodiments, the method effectively reduces the availability of circulating free NBL1 in subjects who have a glomerular disease selected from the group consisting of focal segmental glomerulosclerosis (FSGS), chronic glomerulopathies, hereditary nephritis, and minimal change disease.

In various embodiments, the agent is an agent described in Section 5.3.1, 5.3.2, or 5.3.3 above, incorporated herein by reference.

### 5.5. Methods of Treating Diabetic Kidney Disease or a Glomerular Disease

In a further aspect, methods are presented for treating diabetic kidney disease (DKD) in a subject who has type 1 or type 2 diabetes or who has a glomerular disease. The method comprises administering to the subject an effective amount of an agent capable of inhibiting NBL1 activity.

In some embodiments, the subject has type 1 or type 2 diabetes and one or more of glomerular hypertrophy, glomerulosclerosis, tubulointerstitial inflammation, fibrosis, glomerular hyperfiltration, progressive albuminuria, declining GFR, and ESKD.

In some embodiments, the subject has a glomerular disease. In certain embodiments, the subject has a glomerular disease and does not have type 1 or type 2 diabetes. In certain embodiments, the glomerular disorder is selected from the group consisting of focal segmental glomerulosclerosis (FSGS), chronic glomerulopathies, hereditary nephritis, and minimal change disease.

In various embodiments, the agent is an agent described in Section 5.3.1, 5.3.2, or 5.3.3 above, incorporated here by reference.

### 5.6. Dose Regimen

In some embodiments of the methods described herein, the agent is administered parenterally. In particular embodiments, the agent is administered intravenously. In specific embodiments, the subject is on dialysis and the agent is administered intravenously. In particular embodiments, the agent is administered subcutaneously.

In some embodiments, the agent is administered once. In some embodiments, the agent is administered more than one. In particular embodiments, the agent is administered for at least 3 months. In particular embodiments, the agent is administered for at least 6 months. In particular embodiments, the agent is administered for at least 12 months.

### 5.7. Antibodies and Pharmaceutical Formulations Thereof

In another aspect, antibodies or antigen binding fragments are provided that are capable of binding to NBL1 and inhibiting NBL1-induced toxicity of human podocytes. Embodiments include all antibodies and antigen-binding fragment embodiments described in Section 5.3.2 above, which is incorporated herein by reference.

In a further aspect, pharmaceutical compositions are provided. The pharmaceutical compositions comprise the antibody or antigen binding fragment described herein and a pharmaceutically acceptable carrier. In some embodiments, the composition is formulated for parenteral administration. In some embodiments, the composition is formulated for intravenous administration. In some embodiments, the composition is formulated for subcutaneous administration.

### 5.8. Additional Embodiments

Additional embodiments are set out in the following numbered clauses.
1. A method for delaying onset or progression of kidney damage in a subject who has type 1 diabetes or type 2 diabetes or a glomerular disease, the method comprising:
   administering to the subject an effective amount of an agent capable of inhibiting NBL1 activity.
2. The method of clause 1, wherein the agent is capable of binding to NBL1.
3. The method of clause 2, wherein the agent comprises a bone morphogenetic protein (BMP) or soluble fragment thereof.
4. The method of clause 3, wherein the agent comprises a soluble fragment of human BMP-2.
5. The method of clause 3 or clause 4, wherein the agent further comprises a moiety that extends serum half-life.
6. The method of clause 5, wherein the half-life extension moiety is an antibody Fc domain.
7. The method of clause 5, wherein the half-life extension moiety is at least one covalently linked polyethylene glycol (PEG) moiety.
8. The method of clause 2, wherein the agent comprises an antibody or antigen binding fragment thereof.
9. The method of clause 2, wherein the agent is capable of inhibiting dimerization of NBL1.
10. The method of clause 1, wherein the agent is capable of inhibiting NBL1 expression.
11. The method of any one of clauses 1-10, wherein the subject has type 1 diabetes.
12. The method of any one of clauses 1-10, wherein the subject has type 2 diabetes.
13. The method of any one of clauses 1-10, wherein the subject has a glomerular disease.
14. The method of clause 13, wherein the subject with glomerular disease does not have type 1 diabetes or type 2 diabetes.
15. The method of clause 13 or clause 14, wherein the glomerular disease is selected from the group consisting of focal segmental glomerulosclerosis (FSGS), chronic glomerulopathies, hereditary nephritis, and minimal change disease.
16. The method of clause 15, wherein the glomerular disease is focal segmental glomerulosclerosis.
17. The method of clause 15, wherein the glomerular disease is chronic glomerulopathies.
18. The method of clause 15, wherein the glomerular disease is hereditary nephritis.
19. The method of clause 15, wherein the glomerular disease is minimal change disease.
20. The method of any one of clauses 1-19, wherein the method prevents onset of or slows decline in kidney function.
21. A method of slowing decline in kidney function in a subject who has type 1 or type 2 diabetes or a glomerular disease, the method comprising:
   administering to the subject an effective amount of an agent capable of inhibiting NBL1 activity.
22. The method of clause 21, wherein the method prevents onset of or slows progressive kidney function decline (PKFD).
23. The method of clause 21, wherein the method prevents onset of or slows progression to end-stage kidney disease (ESKD).
24. The method of any one of clauses 21-23, wherein the agent is capable of binding to NBL1.
25. The method of clause 24, wherein the agent comprises a bone morphogenetic protein (BMP) or soluble fragment thereof.
26. The method of clause 25, wherein the agent comprises a soluble fragment of human BMP-2.
27. The method of clause 25 or clause 26, wherein the agent further comprises a moiety that extends serum half-life.
28. The method of clause 27, wherein the half-life extension moiety is an antibody Fc domain.
29. The method of clause 27, wherein the half-life extension moiety is at least one covalently linked polyethylene glycol (PEG) moiety.
30. The method of clause 24, wherein the agent comprises an antibody or antigen binding fragment thereof.
31. The method of clause 24, wherein the agent is capable of inhibiting dimerization of NBL1.
32. The method of any one of clauses 21-23, wherein the agent is capable of inhibiting NBL1 expression.
33. The method of any one of clauses 21-32 wherein the subject has type 1 diabetes.
34. The method of any one of clauses 21-32, wherein the subject has type 2 diabetes.
35. The method of any one of clauses 21-32, wherein the subject has a glomerular disease.
36. The method of clause 35, wherein the subject with glomerular disease does not have type 1 diabetes or type 2 diabetes.
37. The method of clause 35 or clause 36, wherein the glomerular disease is selected from the group consisting of focal segmental glomerulosclerosis (FSGS), chronic glomerulopathies, hereditary nephritis, minimal change disease.
38. The method of clause 37, wherein the glomerular disease is focal segmental glomerulosclerosis.
39. The method of clause 37, wherein the glomerular disease is chronic glomerulopathies.
40. The method of clause 37, wherein the glomerular disease is hereditary nephritis.
41. The method of clause 37, wherein the glomerular disease is minimal change disease.
42. A method of treating diabetic kidney disease (DKD) or a glomerular disease in a subject who has type 1 or type 2 diabetes or a glomerular disease, the method comprising:
   administering to the subject an effective amount of an agent capable of inhibiting NBL1 activity.
43. The method of clause 42, wherein the agent is capable of binding to NBL1.
44. The method of clause 43, wherein the agent comprises a bone morphogenetic protein (BMP) or soluble fragment thereof.
45. The method of clause 44, wherein the agent comprises a soluble fragment of human BMP-2.
46. The method of clause 44 or clause 45, wherein the agent further comprises a moiety that extends serum half-life.
47. The method of clause 46, wherein the half-life extension moiety is an antibody Fc domain.
48. The method of clause 46, wherein the half-life extension moiety is at least one covalently linked polyethylene glycol (PEG) moiety.
49. The method of clause 43, wherein the agent comprises an antibody or antigen binding fragment thereof.
50. The method of clause 43, wherein the agent is capable of inhibiting dimerization of NBL1.
51. The method of clause 42, wherein the agent is capable of inhibiting NBL1 expression.
52. The method of any one of clauses 42-51, wherein the subject has type 1 diabetes.
53. The method of any one of clauses 42-51, wherein the subject has type 2 diabetes.
54. The method of any one of clauses 42-51, wherein the subject has a glomerular disease.
55. The method of clause 54, wherein the subject with glomerular disease does not have type 1 diabetes or type 2 diabetes.
56. The method of clause 54 or clause 55, wherein the glomerular disease is selected from the group consisting of focal segmental glomerulosclerosis, chronic glomerulopathies, hereditary nephritis, minimal change disease.
57. The method of clause 56, wherein the glomerular disease is focal segmental glomerulosclerosis.
58. The method of clause 56, wherein the glomerular disease is chronic glomerulopathies.
59. The method of clause 56, wherein the glomerular disease is hereditary nephritis.
60. The method of clause 56, wherein the glomerular disease is minimal change disease.
61. The method of any one of clauses 1-60, wherein the agent is administered parenterally.
62. The method of any one of clauses 1-61, wherein the agent is administered for at least 3 months.
63. The method of clause 62, wherein the agent is administered for at least 6 months.
64. The method of clause 63, wherein the agent is administered for at least 12 months.

### 6. EXAMPLES

Below are examples of specific embodiments for carrying out the present disclosure. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology. Such techniques are explained fully in the literature.

### 6.1. Example 1. NBL1 is Directly Toxic to Podocytes and Renal Tubular Cells

Increased DAN protein (Grem1, Grem2, Grem3, Cerberus, NBL1, SOST, and USG1) levels have been associated with severe disease-states in adult kidneys. Wen et al., Biochimie 160:113-121 (2019).

To assess whether the increased presence of a DAN protein has a causal effect on kidney damage, we cultured human podocytes (HuPodo), human mesangial cells (HuHMRC), and human renal tubular cells (HuK2) in vitro for 48 hours in the presence or absence of human NBL1. Human umbilical vein endothelial cells (Huvec) were used as a control. Podocytes were cultured in increasing concentrations of NBL1: 0.2 μg/ml, 1.0 μg/ml, and 2.0 μg/ml. Mesangial cells and tubular cells were cultured in 2 μg/ml NBL1. Human umbilical vein endothelial cells were cultured in increasing concentrations of 0.2 μg/ml, 2 μg/ml, and 10 μg/ml NBL1.

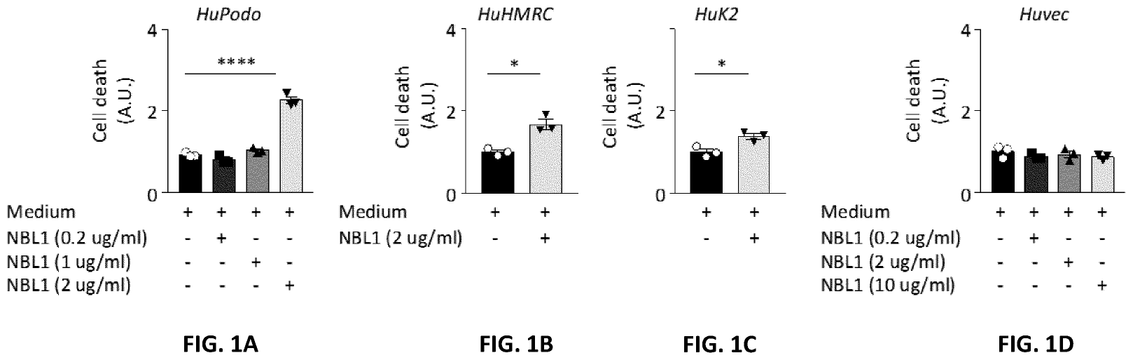

FIGS. 1A-1D are bar graphs summarizing cell death analysis data, quantified using arbitrary units (AU) for convenience. The results show increased apoptosis/death of human podocytes, and to a lesser degree mesangial and tubular cells, directly correlated to the added presence of NBL1 to culture media. Cell death was undetectable in NBL1-cultured human umbilical vein endothelial cells (Huvec), even at higher concentrations.

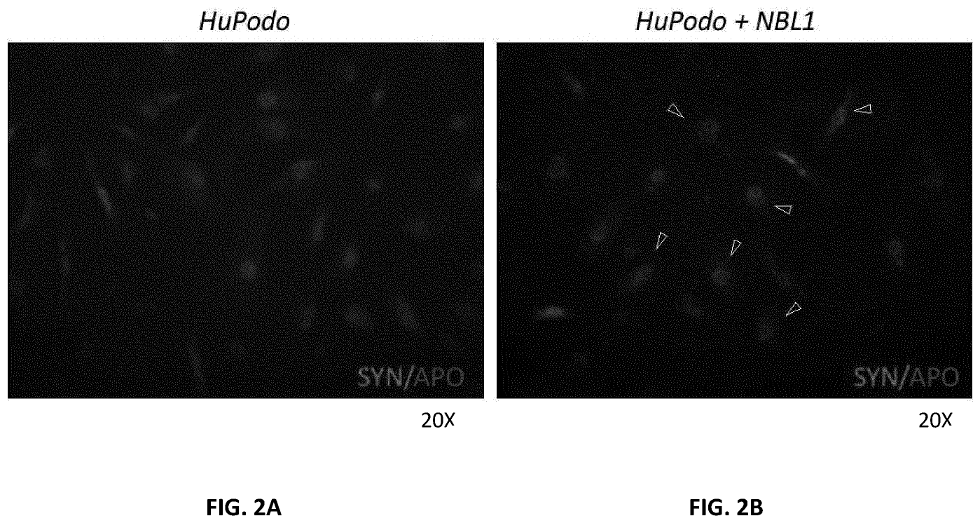

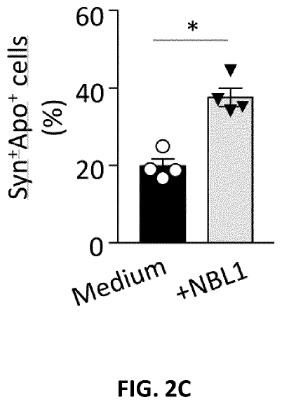

FIGS. 2A-2B show representative images of confocal analysis conducted on human podocytes left untreated (FIG. 2A) or cultured with NBL1 at 2 μg/ml (FIG. 2B), stained with Synaptopodin and Apoptag. Merged pictures are presented. These micrographs show evidence of NBL1-induced apoptosis of human podocytes. The arrows highlight the colocalization of Apoptag and Synaptopodin, which are markers for apoptosis and podocytes respectively. The image emphasizes that the majority of podocytes are undergoing apoptosis. FIG. 2C is a bar graph quantifying the percentage of Apoptag⁺Synaptopodyn⁺ double positive human podocytes in the presence/absence of NBL1 (n=3). The results indicate increased level of apoptotic differentiated human podocytes in the presence of NBL1.

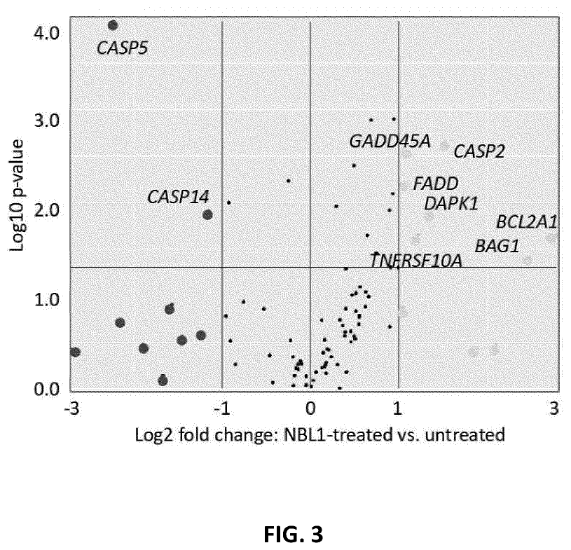

FIG. 3 shows transcriptome analysis of apoptosis-related genes of human podocytes cultured with NBL1 at 2 μg/ml or left untreated. The data show significant increase in expression of apoptosis-related genes upon exposure to NBL-1 in vitro.

Collectively, these experiments show that NBL1 is directly toxic to podocytes and renal tubular cells, with exposure to NBL1 causing apoptosis of human podocytes and renal tubular cells.

### 6.2. Example 2. BMP Proteins are not Expressed in and are not Secreted by Kidney Cells In Vitro

Like other DAN proteins, NBL1 is known to interact with bone morphogenetic proteins. Hung et al., Biol. of Reprod. (2012) 86 (5): 158, 1-9.

To determine whether NBL1 exerts its toxicity through inhibition of BMPs within the kidney, we analyzed whether BMP2, BMP4 and BMP7 were expressed as mRNA in human podocytes. We did not detect any mRNA expression of any of the BMPs. Next, we assessed whether BMP2, BMP4 or BMP7 are secreted from podocytes into the supernatant, thus allowing an autocrine/paracrine pro-survival effect. We did not detect any of the BMPs in the supernatants of human podocytes, thus confirming that podocytes are not able to synthesize and secrete BMPs in vitro (data not shown).

The toxic effect of NBL1 on podocytes is thus independent of the presence of BMPs; NBL1's pro-apoptotic effect is not mediated via inhibition of BMPs.

### 6.3. Example 3. NBL1 is not Expressed in Kidney Cells, but is Expressed in Circulating Immune Cells, Intestinal and Muscle Tissue

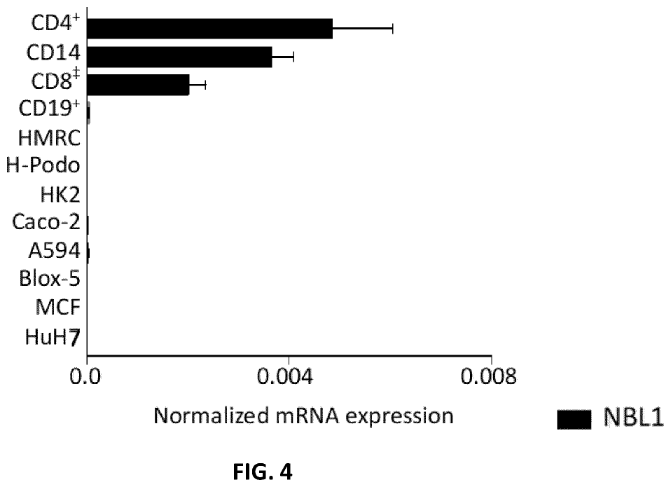

Having determined that NBL1 is directly toxic to podocytes and renal tubular cells, we sought its physiological origin by measuring mRNA expression of NBL1 in a number of human cell lines, including kidney cell lines, using RT-PCR with expression normalized to beta actin expression. FIG. 4 shows that NBL1 was undetectable in kidney-derived cells. NBL1 was also undetectable in various other cell lines. However, it was highly expressed in immune cells, such as CD14⁺ monocytes and CD4⁺ and CD8⁺ T cells (see FIG. 4).

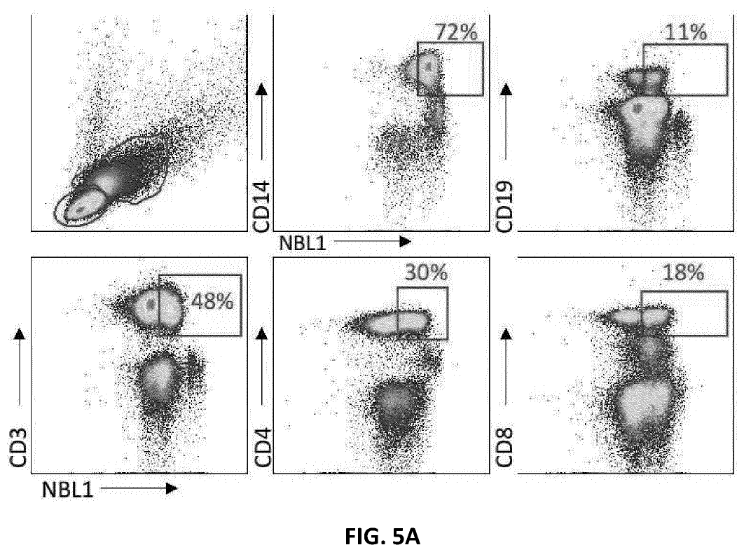

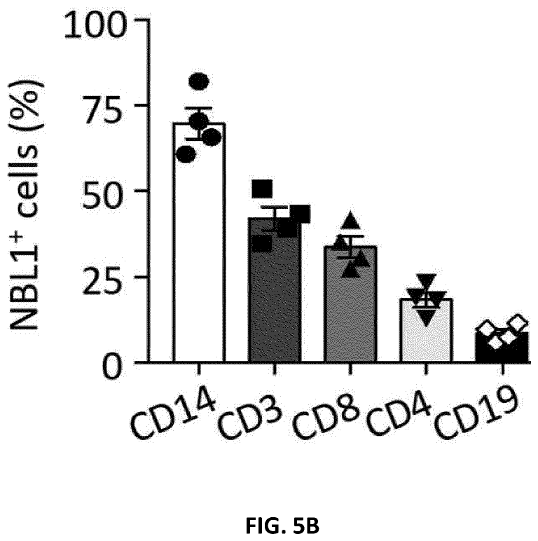

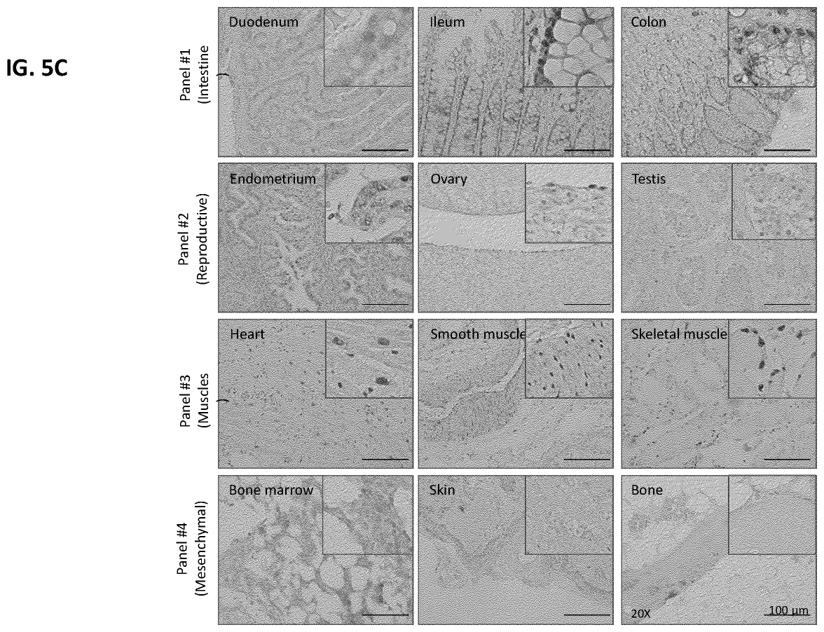

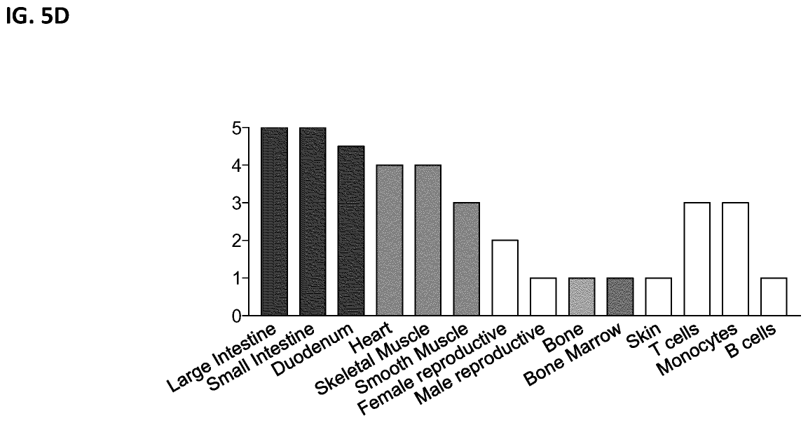

Additional analysis using flow-cytometry of peripheral blood mononuclear cells (PBMCs) isolated from blood samples of healthy volunteers confirmed high expression of NBL1 protein in myeloid (CD14⁺) cells and various T cell subsets (CD3⁺, CD4⁺ and CD8⁺ T cells) (see FIGS. 5A-5B). Human PBMCs were purified from 8 ml blood samples collected from non-diabetic subjects (n=5) at the ASST Sacco-FBF in Milan (Italy) by using Lymphoprep (07801, Stem Cell Technologies, Cambridge, MA) and cells were then stained for flow-cytometry analysis with anti-human CD3 (300330), anti-CD45 (560178), anti-CD4 (561030), anti-CD8 (560774), anti-CD14 (561707) from Biolegend (San Diego, CA) and BD Biosciences (San Jose, CA) to quantify surface expression. Rabbit polyclonal anti-NBL1 (Sigma, HPA007394) followed by donkey anti-rabbit AlexaFluor488 antibody (ThermoFisher Scientific) was used to stain for NBL1. Cells were analyzed using a BD FACS Celesta (BD Biosciences).

To further explore the expression of NBL1, a comprehensive immunostaining study of NBL1 human tissue expression was performed. In this study, NBL1 expression was detected by immunohistochemistry and flow cytometry on human tissue specimens. NBL1 immunoreactivity was semi-quantitatively scored based on the percentage of NBL1 positive cells in the total cells per region of interest. The scoring was as follows: <10%, 1+; 10-30%, 2+; 30-50%, 3+; 50-80%, 4+, and >80%, 5+. Archival formalin-fixed, paraffin-embedded tissue samples of non-diabetic subjects from Pathology Unit, University of Parma were used for NBL1 immunostaining (anti-NBL1 primary antibody #HPA007394, Merck). A further magnification of each panel is shown in the insert on the top right black square, in which black arrows highlight the positive staining. Original magnification 20×, scale bar 100 μm (See FIG. 5C).

A semi-quantitative score was also applied to NBL1 expression detected in T cells (CD3⁺NBL1⁺), B cells (CD19⁺NBL1⁺) and monocytes (CD14⁺NBL1⁺) (See FIG. 5A) based on the quantification of percentage of double positive cells as follows: <15%, 1+; 15-45%, 2+; 45-75%, 3+; 75-90%, 4+, and >90%, 5+.

The semi-quantitative scores illustrating NBL1 protein expression in human tissues are illustrated in the bar graph presented in FIG. 5D. For histocytochemistry, the mean value of the score calculated in n=3 separate slides are presented. For flow cytometry, scores representing T cells, B cells and monocytes, mean values obtained in n=5 samples analyzed are presented. (See FIG. 5D).

The results of the study demonstrate that NBL1, which is not expressed in kidney, is expressed in circulatory immune cells, notably T cells and monocytes, in various intestinal tissues, and muscle, with lesser expression in reproductive tissues, bone and bone marrow.

### 6.4. Example 4. Neutralizing NBL1 with an Antagonist Prevents Toxicity

Having shown that NBL1 is toxic to podocytes and renal tubular cells, that this toxicity is directly mediated, and that NBL1 is not produced locally in renal cells but is instead expressed in circulating immune cells and other non-renal tissues, we tested whether NBL1 could be an appropriate target for direct therapeutic intervention to protect kidney cells from damage.

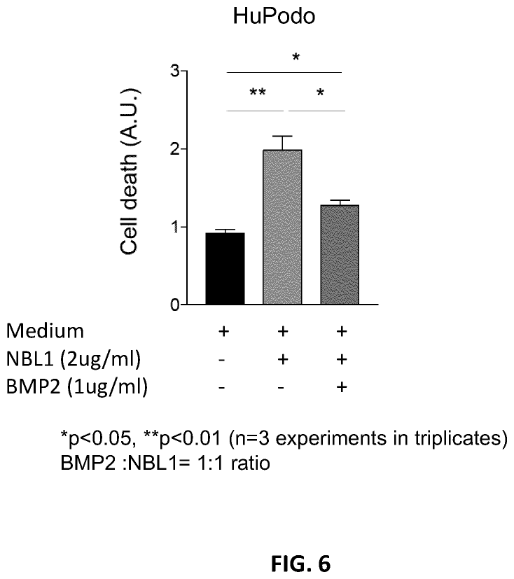

Human podocytes were cultured for 48 hours in the presence of NBL1 (2 μg/ml) in the presence or absence of soluble BMP2 in a 1:1 ratio (NBL1: sBMP2=1:1). Cell death was detected by ELISA. Incubation with soluble BMP2 significantly reduced the cell death effects experienced by human podocytes cultured in the presence of NBL1, indicating that soluble BMP2 is capable of partially neutralizing NBL1's pro-apoptotic effect on human podocytes in vitro. Results for the above experiments are shown in FIG. 6. Three independent experiments were run in duplicate. Data are presented as mean±SEM. A commercial anti-NBL1 tool antibody (Sigma) showed a smaller effect (data not shown).

### 6.5. Example 5. Human Anti-NBL1 Antibody Discovery Campaign

Naïve human phage display libraries were panned to discover Fabs capable of binding human NBL1 (UniProt ID P41271), and further screened for potential inter species cross reactivity to mouse NBL1 (UniProt ID Q61477) and/or cynomolgus (“cyno”) NBL1 (UniProt ID A0A2K5WIY3). The top 25 Fabs were cloned into expression vectors and expressed as full length human IgG1 antibodies. Antigen binding of the IgG1 formatted antibodies was tested by ELISA and EC50 values were calculated. Twenty (20) IgG1 antibodies were shown to bind to human, mouse, and cyno NBL1 with EC50 in the ELISA of between 10 ng/mL and 100 ng/ml (˜0 to 600 pM).

Tables presented in Section 5.3.2 above provide the VH and VL sequences, and separately, the CDR sequences, of the 20 IgG1 antibodies.

### 6.6. Example 6. Monoclonal Anti-NBL1 Antibodies Rescue NBL1-Mediated Apoptosis of Human Podocytes In Vitro

Having demonstrated that NBL1 is directly toxic to podocytes and renal tubular cells, and that the toxicity can be averted using sBMP2, which is capable of specific binding to NBL1, we decided to assess the effects of monoclonal anti-NBL1 antibodies on the cell death effects experienced by human podocytes cultured in the presence of NBL1. Of the monoclonal antibodies generated in Example 6, we tested 16 antibodies and assessed their ability to prevent cell death and apoptosis in human podocytes cultured in vivo.

Human podocytes were cultured in RPMI supplemented with 10% FBS and with ITS (1×). Human NBL1 recombinant protein was obtained from Genscript (Piscataway, NJ). Human monoclonal NBL1 antibodies obtained by cloning NBL1-binding phage-displayed Fabs into a human IgG1 format (Example 6 above) were tested at a concentration of 20 μg/ml.

The human podocytes were cultured for 48 hours in the presence/absence of human NBL1 (2 μg/ml) and in the presence/absence of our generated anti-NBL1 mAbs (20 μg/ml) at a ratio of 1:1 (mAbs: NBL1). Cell lysates were collected at 48 hours of culture, and cell death/apoptosis was assessed by using ELISA (Roche Diagnostics GmbH, 11544675001, Mannheim, Germany). Quantification of cell death was normalized to untreated cells.

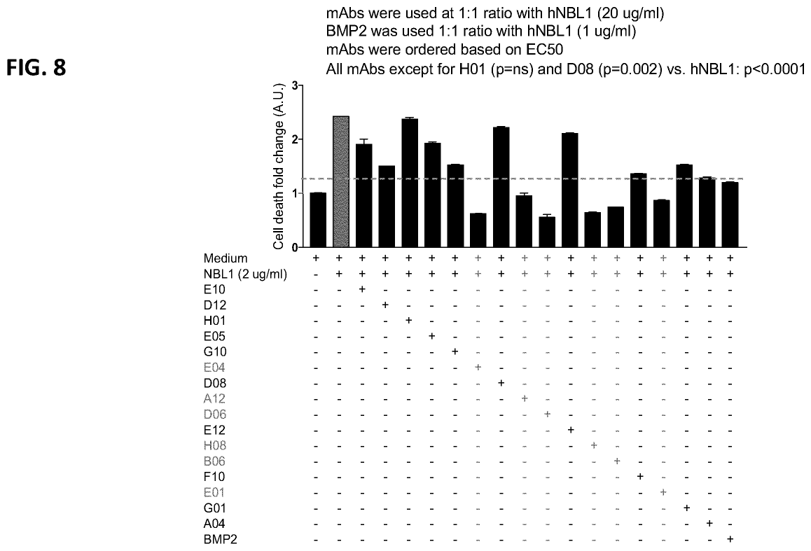

We observed that following incubation in the presence of these newly generated mAbs, a number of the mAbs were able to prevent/reduce NBL1-induced cell death (p<0.0001) to varying degrees. Most importantly, 6 out of the 16 mAbs (YU1018-H08, YU1019-B06, YU1018-E01, YU1018-E04, YU1019-A12, YU1018-D06) were able to reduce the cell death effects experienced by human podocytes cultured in the presence of NBL1 to levels at or below the negative control (left-most bar, “Medium”), demonstrating that these mAbs are capable of completely neutralizing NBL1's pro-apoptotic effect on human podocytes in vitro, which further indicates that among the tested antibodies, these antibodies will most potently be able to reduce progression of glomerular/renal disease in subjects with elevated levels of circulating NBL1, including human subjects with TID and T2D. Results are shown in FIG. 8.

In the same experiment, we tested the ability of soluble BMP2 (1 μg/ml, at a 1:1 BMP2: NBL1 ratio), to neutralize the effects of NBL1. As shown in FIG. 8, mAbs H08, B06, and E01 were significantly more robust in the reduction of NBL1-mediated apoptotic effects on human podocytes than sBMP2. This highlights the beneficial effectiveness of anti-NBL1 antibodies in protecting podocytes from NBL1-mediated toxicity and damage.

### 6.7. Example 7. NBL1 is Elevated in Type 1 Diabetes and Type 2 Diabetes

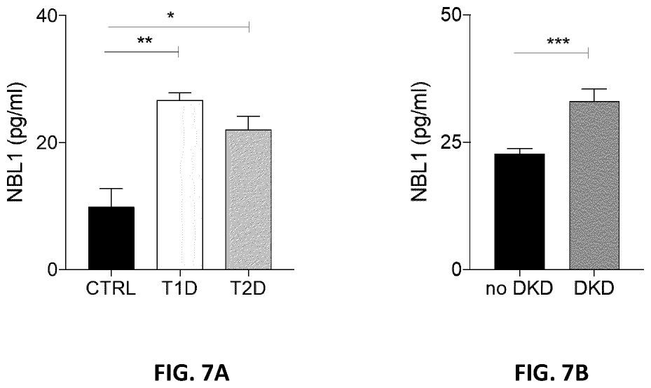

In order to determine whether NBL1 contributes to kidney damage in diabetes, NBL1 serum levels were assessed using an immunotargeted assay. NBL1 serum levels were measured in patients with long-standing type 1 diabetes (TID, n=150) and compared with NBL1 serum levels observed in sera of non-diabetic subjects (n=15). NBL1 serum levels of patients with long-standing type 1 diabetes displayed a significant increase in NBL1. A parallel comparison of NBL1 serum levels in patients with type 2 diabetes (T2D, n=70) versus levels in non-diabetic subjects also displayed a significant increase in NBL1. Results are shown in FIG. 7A.

Subsequently, patients with TID or T2D, and who had also received a diagnosis of diabetic kidney disease (DKD), were selected and we analyzed their NBL1 serum levels. The analysis demonstrated that patients with chronic kidney disease (CKD) at stage 2-3 (eGFR<60 ml/min/m², n=60) had a 3-fold increase in NBL1 serum levels as compared to non-diabetic subjects and a 2-fold increase in NBL1 serum levels as compared to diabetic patients without CKD (see FIG. 7B).

Inhibition of NBL1 is therefore a new therapeutic approach for preventing onset and progression of kidney damage in a patient with type 1 or type 2 diabetes. Inhibition of NBL1 will also be effective in treating non-diabetes glomerular diseases in which damage is mediated by NBL1.

### 7. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents, and patent applications cited within the body of the instant specification are hereby incorporated by reference in their entirety, for all purposes.

1. A method for delaying onset or progression of kidney damage in a subject who has type 1 diabetes or type 2 diabetes or a glomerular disease, the method comprising:
administering to the subject an effective amount of an agent capable of inhibiting NBL1 activity.

2-41. (canceled)

42. An antibody or antigen binding fragment, wherein the antibody is capable of binding to NBL1 and inhibiting NBL1-induced toxicity of human podocytes.

43. The antibody of claim 42, wherein the antibody or antigen-binding fragment comprises three heavy chain CDRs and three light chain CDRs having sequences selected from:
a) SEQ ID NOs: 3, 8, and 13 and SEQ ID NOs: 18, 23, and 28 (antibody E05a);
b) SEQ ID NOs: 33, 38, and 43 and SEQ ID NOs: 48, 53, and 58 (antibody H08);
c) SED ID NOs: 63, 68, and 73 and SED ID NOs: 78, 83, and 88 (antibody F06);
d) SEQ ID NOs: 93, 98, and 103 and SEQ ID NOs: 108, 113, and 118 (antibody A12);
e) SED ID NOs: 123, 128, and 133 and SED ID NOs: 138, 143, and 148 (antibody G01);
f) SED ID NOs: 153, 158, and 163 and SED ID NOs: 168, 173 and 178 (antibody E11);
g) SEQ ID NOs: 183, 188, and 193 and SEQ ID NOs: 198, 203, and 208 (antibody B06);
h) SED ID NOs: 213, 218, and 223 and SED ID NOs: 228, 233, and 238 (antibody D12);
i) SED ID NOs: 243, 248, and 253 and SED ID NOs: 258, 263 and 268 (antibody H01);
j) SED ID NOs: 273, 278, and 283 and SED ID NOs: 288, 293, and 298 (antibody C11);
k) SED ID NOs: 303, 308, and 313 and SED ID NOs: 318, 323, and 328 (antibody E05b);
l) SED ID NOs: 333, 338, and 343 and SED ID NOs: 348, 353 and 358 (antibody F10);
m) SED ID NOs: 363, 368 and 373 and SED ID NOs: 378, 383 and 388 (antibody G10);
n) SEQ ID NOs: 393, 398, and 403 and SEQ ID NOs: 408, 413, and 418 (antibody E04);
o) SED ID NOs: 423, 428, and 433 and SED ID NOs: 438, 443, and 448 (antibody E07);
p) SED ID NOs: 453, 458, and 463 and SED ID NOs: 468, 473, and 478 (antibody E12);
q) SED ID NOs: 483, 488, and 493 and SED ID NOs: 498, and 503, and 508 (antibody D08);
r) SED ID NOs: 513, 518, and 523 and SED ID NOs: 528, 533, and 538 (antibody E10);
s) SEQ ID NOs: 543, 548, and 553 and SEQ ID NOs: 558, 563, and 568 (antibody D06); and
t) SED ID NOs: 573, 578, and 583 and SED ID NOs: 588, 593, and 598 (antibody E01),
or having sequences that differ from the selected CDR sequences (a)-(t) by at most two conservative amino acid substitutions in each CDR.

44. The antibody or antigen binding fragment of claim 43, wherein the antibody or antigen-binding fragment comprises three heavy chain CDRs and three light chain CDRs with sequences identical to the selected CDRs.

45. The antibody or antigen binding fragment of claim 43, wherein the three heavy chain CDRs and three light chain CDRs have sequences selected from:
b) SEQ ID NOs: 33, 38, and 43 and SEQ ID NOs: 48, 53 and 58 (antibody H08);
d) SEQ ID NOs: 93, 98, and 103 and SEQ ID NOs: 108, 113, and 118 (antibody A12);
g SEQ ID NOs: 183, 188, and 193 and SEQ ID NOs: 198, 203, and 208 (antibody B06);
n) SEQ ID NOs: 393, 398, and 403 and SEQ ID NOs: 408, 413, and 418 (antibody E04);
s) SEQ ID NOs: 543, 548, and 553 and SEQ ID NOs: 558, 563, and 568 (antibody D06); and
t) SEQ ID NOs: 573, 578, and 583 and SEQ ID NOs: 588, 593, and 598 (antibody E01),
or having sequences that differ from the selected CDR sequences b), d), g), n) s), and t) by at most two conservative amino acid substitutions in each CDR.

46. The antibody or antigen binding fragment of claim 45, wherein the antibody or antigen-binding fragment comprises three heavy chain CDRs and three light chain CDRs with sequences identical to the selected CDRs.

47. The antibody or antigen binding fragment of claim 46, wherein the antibody framework regions are human antibody framework regions.

48. The antibody or antigen binding fragment of claim 47, wherein the antibody is a full length bivalent monospecific monoclonal antibody.

49. The antibody or antigen binding fragment of claim 48, wherein the antibody comprises human IgG1, IgG2, or IgG4 heavy chain constant regions.

50. The antibody or antigen binding fragment of claim 49, wherein the antibody comprises a human IgG1 constant region.

51. The antibody or antigen binding fragment of claim 50, wherein the antibody Fc region has engineered mutations that reduce antibody binding to FcRγ and/or reduce complement fixation.

52. The antibody or antigen binding fragment of claim 47, wherein the antibody is a Fab, optionally wherein the Fab is PEGylated.

53. The antibody or antigen binding fragment of claim 47, wherein the antibody or antigen binding fragment is further capable of binding to cynomolgus monkey NBL1.

54. The antibody or antigen binding fragment of claim 47, wherein the antibody or antigen binding fragment is further capable of binding to mouse NBL1.

55. The antibody or antigen binding fragment of claim 49, wherein the antibody or antigen binding fragment has a binding affinity (K_{D}) for human NBL1 of less than 100 nM.

56. The antibody or antigen binding fragment of claim 55, wherein the antibody or antigen binding fragment has a binding affinity (K_{D}) for human NBL1 of less than 10 nM.

57. The antibody or antigen binding fragment of claim 55, wherein the antibody or antigen binding fragment has a binding affinity (K_{D}) for human NBL1 of less than 5 nM.

58. A pharmaceutical composition, comprising:
an antibody or antigen binding fragment of, and
a pharmaceutically acceptable carrier, wherein
the anybody or antigen binding fragment is capable of binding to NBL1.

59. The pharmaceutical composition of claim 58, wherein the composition is formulated for parenteral administration.

60. The pharmaceutical composition of claim 59, wherein the composition is formulated for intravenous administration.

61. The pharmaceutical composition of claim 59, wherein the composition is formulated for subcutaneous administration.

62. The pharmaceutical composition of claim 58, wherein the antibody or antigen binding fragment comprises three heavy chain CDRs and three light chain CDRs
having sequences selected from:
a) SEQ ID NOs: 3, 8, and 13 and SEQ ID NOs: 18, 23, and 28 (antibody E05a);
b) SEQ ID NOs: 33, 38, and 43 and SEQ ID NOs: 48, 53, and 58 (antibody H08);
c) SED ID NOs: 63, 68, and 73 and SED ID NOs: 78, 83, and 88 (antibody F06);
d) SEQ ID NOs: 93, 98, and 103 and SEQ ID NOs: 108, 113, and 118 (antibody A12);
e) SED ID NOs: 123, 128, and 133 and SED ID NOs: 138, 143, and 148 (antibody G01);
f) SED ID NOs: 153, 158, and 163 and SED ID NOs: 168, 173 and 178 (antibody E11);
g) SEQ ID NOs: 183, 188, and 193 and SEQ ID NOs: 198, 203, and 208 (antibody B06);
h) SED ID NOs: 213, 218, and 223 and SED ID NOs: 228, 233, and 238 (antibody D12);
i) SED ID NOs: 243, 248, and 253 and SED ID NOs: 258, 263 and 268 (antibody H01);
j) SED ID NOs: 273, 278, and 283 and SED ID NOs: 288, 293, and 298 (antibody C11);
k) SED ID NOs: 303, 308, and 313 and SED ID NOs: 318, 323, and 328 (antibody E05b);
l) SED ID NOs: 333, 338, and 343 and SED ID NOs: 348, 353 and 358 (antibody F10);
m) SED ID NOs: 363, 368 and 373 and SED ID NOs: 378, 383 and 388 (antibody G10);
n) SEQ ID NOs: 393, 398, and 403 and SEQ ID NOs: 408, 413, and 418 (antibody E04);
o) SED ID NOs: 423, 428, and 433 and SED ID NOs: 438, 443, and 448 (antibody E07);
p) SED ID NOs: 453, 458, and 463 and SED ID NOs: 468, 473, and 478 (antibody E12);
q) SED ID NOs: 483, 488, and 493 and SED ID NOs: 498, and 503, and 508 (antibody D08);
r) SED ID NOs: 513, 518, and 523 and SED ID NOs: 528, 533, and 538 (antibody E10);
s) SEQ ID NOs: 543, 548, and 553 and SEQ ID NOs: 558, 563, and 568 (antibody D06); and
t) SED ID NOs: 573, 578, and 583 and SED ID NOs: 588, 593, and 598 (antibody E01),
or having sequences that differ from the selected CDR sequences (a)-(t) by at most two conservative amino acid substitutions in each CDR.


SEQUENCE LISTING (704 sequences)

SEQ ID NO: 1 - protein, 9 aa, synthetic construct
GGSISSGGY

SEQ ID NO: 2 - protein, 12 aa, synthetic construct
GGSISSGGYYWS

SEQ ID NO: 3 - protein, 7 aa, synthetic construct
SGGYYWS

SEQ ID NO: 4 - protein, 8 aa, synthetic construct
SSGGYYWS

SEQ ID NO: 5 - protein, 10 aa, synthetic construct
GGSISSGGYY

SEQ ID NO: 6 - protein, 5 aa, synthetic construct
YYSGS

SEQ ID NO: 7 - protein, 9 aa, synthetic construct
YIYYSGSTY

SEQ ID NO: 8 - protein, 16 aa, synthetic construct
YIYYSGSTYYNPSLKS

SEQ ID NO: 9 - protein, 12 aa, synthetic construct
WIGYIYYSGSTY

SEQ ID NO: 10 - protein, 7 aa, synthetic construct
IYYSGST

SEQ ID NO: 11 - protein, 10 aa, synthetic construct
TNSRITMIAD

SEQ ID NO: 12 - protein, 10 aa, synthetic construct
TNSRITMIAD

SEQ ID NO: 13 - protein, 10 aa, synthetic construct
TNSRITMIAD

SEQ ID NO: 14 - protein, 11 aa, synthetic construct
ARTNSRITMIA

SEQ ID NO: 15 - protein, 12 aa, synthetic construct
ARTNSRITMIAD

SEQ ID NO: 16 - protein, 13 aa, synthetic construct
SGSSSNIGNNYVS

SEQ ID NO: 17 - protein, 13 aa, synthetic construct
SGSSSNIGNNYVS

SEQ ID NO: 18 - protein, 13 aa, synthetic construct
SGSSSNIGNNYVS

SEQ ID NO: 19 - protein, 9 aa, synthetic construct
IGNNYVSWY

SEQ ID NO: 20 - protein, 8 aa, synthetic construct
SSNIGNNY

SEQ ID NO: 21 - protein, 7 aa, synthetic construct
DNNKRPS

SEQ ID NO: 22 - protein, 7 aa, synthetic construct
DNNKRPS

SEQ ID NO: 23 - protein, 7 aa, synthetic construct
DNNKRPS

SEQ ID NO: 24 - protein, 10 aa, synthetic construct
LLIYDNNKRP

SEQ ID NO: 25 - skipped

SEQ ID NO: 26 - protein, 11 aa, synthetic construct
GTWDSSLSAGV

SEQ ID NO: 27 - protein, 11 aa, synthetic construct
GTWDSSLSAGV

SEQ ID NO: 28 - protein, 11 aa, synthetic construct
GTWDSSLSAGV

SEQ ID NO: 29 - protein, 10 aa, synthetic construct
GTWDSSLSAG

SEQ ID NO: 30 - protein, 11 aa, synthetic construct
GTWDSSLSAGV

SEQ ID NO: 31 - protein, 9 aa, synthetic construct
GGSISSGGY

SEQ ID NO: 32 - protein, 9 aa, synthetic construct
GGSISSGGY

SEQ ID NO: 33 - protein, 7 aa, synthetic construct
SGGYYWS

SEQ ID NO: 34 - protein, 8 aa, synthetic construct
SSGGYYWS

SEQ ID NO: 35 - protein, 10 aa, synthetic construct
GGSISSGGYY

SEQ ID NO: 36 - protein, 5 aa, synthetic construct
YYSGS

SEQ ID NO: 37 - protein, 9 aa, synthetic construct
YIYYSGSTY

SEQ ID NO: 38 - protein, 16 aa, synthetic construct
YIYYSGSTYYNPSLKS

SEQ ID NO: 39 - protein, 12 aa, synthetic construct
WIGYIYYSGSTY

SEQ ID NO: 40 - protein, 7 aa, synthetic construct
IYYSGST

SEQ ID NO: 41 - protein, 10 aa, synthetic construct
TNSRITMIAD

SEQ ID NO: 42 - protein, 10 aa, synthetic construct
TNSRITMIAD

SEQ ID NO: 43 - protein, 10 aa, synthetic construct
TNSRITMIAD

SEQ ID NO: 44 - protein, 11 aa, synthetic construct
ARTNSRITMIA

SEQ ID NO: 45 - protein, 12 aa, synthetic construct
ARTNSRITMIAD

SEQ ID NO: 46 - protein, 13 aa, synthetic construct
SGSSSNIGNNYVS

SEQ ID NO: 47 - protein, 13 aa, synthetic construct
SGSSSNIGNNYVS

SEQ ID NO: 48 - protein, 13 aa, synthetic construct
SGSSSNIGNNYVS

SEQ ID NO: 49 - protein, 9 aa, synthetic construct
IGNNYVSWY

SEQ ID NO: 50 - protein, 8 aa, synthetic construct
SSNIGNNY

SEQ ID NO: 51 - protein, 7 aa, synthetic construct
DNNKRPS

SEQ ID NO: 52 - protein, 7 aa, synthetic construct
DNNKRPS

SEQ ID NO: 53 - protein, 7 aa, synthetic construct
DNNKRPS

SEQ ID NO: 54 - protein, 10 aa, synthetic construct
LLTYDNNKRP

SEQ ID NO: 55 - skipped

SEQ ID NO: 56 - protein, 12 aa, synthetic construct
GTWDSSLSAGHV

SEQ ID NO: 57 - protein, 12 aa, synthetic construct
GTWDSSLSAGHV

SEQ ID NO: 58 - protein, 12 aa, synthetic construct
GTWDSSLSAGHV

SEQ ID NO: 59 - protein, 11 aa, synthetic construct
GTWDSSLSAGH

SEQ ID NO: 60 - protein, 12 aa, synthetic construct
GTWDSSLSAGHV

SEQ ID NO: 61 - protein, 7 aa, synthetic construct
GFTFSSY

SEQ ID NO: 62 - protein, 10 aa, synthetic construct
GFTFSSYAMS

SEQ ID NO: 63 - protein, 5 aa, synthetic construct
SYAMS

SEQ ID NO: 64 - protein, 6 aa, synthetic construct
SSYAMS

SEQ ID NO: 65 - protein, 8 aa, synthetic construct
GFTFSSYA

SEQ ID NO: 66 - protein, 6 aa, synthetic construct
SGSGGS

SEQ ID NO: 67 - protein, 10 aa, synthetic construct
AISGSGGSTY

SEQ ID NO: 68 - protein, 17 aa, synthetic construct
AISGSGGSTYYADSVKG

SEQ ID NO: 69 - protein, 13 aa, synthetic construct
WVSAISGSGGSTY

SEQ ID NO: 70 - protein, 8 aa, synthetic construct
ISGSGGST

SEQ ID NO: 71 - protein, 16 aa, synthetic construct
GSPYYYDSSGYYPLDY

SEQ ID NO: 72 - protein, 16 aa, synthetic construct
GSPYYYDSSGYYPLDY

SEQ ID NO: 73 - protein, 16 aa, synthetic construct
GSPYYYDSSGYYPLDY

SEQ ID NO: 74 - protein, 17 aa, synthetic construct
AKGSPYYYDSSGYYPLD

SEQ ID NO: 75 - protein, 18 aa, synthetic construct
AKGSPYYYDSSGYYPLDY

SEQ ID NO: 76 - protein, 18 aa, synthetic construct
AKGSPYYYDSSGYYPLDY

SEQ ID NO: 77 - protein, 16 aa, synthetic construct
RSSQSLLHSNGYNYLD

SEQ ID NO: 78 - protein, 16 aa, synthetic construct
RSSQSLLHSNGYNYLD

SEQ ID NO: 79 - protein, 12 aa, synthetic construct
LHSNGYNYLDWY

SEQ ID NO: 80 - protein, 11 aa, synthetic construct
QSLLHSNGYNY

SEQ ID NO: 81 - protein, 7 aa, synthetic construct
LGSNRAP

SEQ ID NO: 82 - protein, 7 aa, synthetic construct
LGSNRAP

SEQ ID NO: 83 - protein, 7 aa, synthetic construct
LGSNRAP

SEQ ID NO: 84 - protein, 10 aa, synthetic construct
LLIYLGSNRA

SEQ ID NO: 85 - skipped

SEQ ID NO: 86 - protein, 9 aa, synthetic construct
MQALQTPLT

SEQ ID NO: 87 - protein, 9 aa, synthetic construct
MQALQTPLT

SEQ ID NO: 88 - protein, 9 aa, synthetic construct
MQALQTPLT

SEQ ID NO: 89 - protein, 8 aa, synthetic construct
MQALQTPL

SEQ ID NO: 90 - protein, 9 aa, synthetic construct
MQALQTPLT

SEQ ID NO: 91 - protein, 9 aa, synthetic construct
GGSISSGDY

SEQ ID NO: 92 - protein, 12 aa, synthetic construct
GGSISSGDYYWS

SEQ ID NO: 93 - protein, 7 aa, synthetic construct
SGDYYWS

SEQ ID NO: 94 - protein, 8 aa, synthetic construct
SSGDYYWS

SEQ ID NO: 95 - protein, 10 aa, synthetic construct
GGSISSGDYY

SEQ ID NO: 96 - protein, 5 aa, synthetic construct
YYSGS

SEQ ID NO: 97 - protein, 9 aa, synthetic construct
YIYYSGSTY

SEQ ID NO: 98 - protein, 16 aa, synthetic construct
YIYYSGSTYYNPSLKS

SEQ ID NO: 99 - protein, 12 aa, synthetic construct
WIGYIYYSGSTY

SEQ ID NO: 100 - protein, 7 aa, synthetic construct
IYYSGST

SEQ ID NO: 101 - protein, 8 aa, synthetic construct
GRRLLLFH

SEQ ID NO: 102 - protein, 8 aa, synthetic construct
GRRLLLFH

SEQ ID NO: 103 - protein, 8 aa, synthetic construct
GRRLLLFH

SEQ ID NO: 104 - protein, 9 aa, synthetic construct
ARGRRLLLF

SEQ ID NO: 105 - protein, 10 aa, synthetic construct
ARGRRLLLFH

SEQ ID NO: 106 - protein, 13 aa, synthetic construct
SGSGSSIGNNYVS

SEQ ID NO: 107 - protein, 13 aa, synthetic construct
SGSGSSIGNNYVS

SEQ ID NO: 108 - protein, 13 aa, synthetic construct
SGSGSSIGNNYVS

SEQ ID NO: 109 - protein, 9 aa, synthetic construct
IGNNYVSWY

SEQ ID NO: 110 - protein, 8 aa, synthetic construct
GSSIGNNY

SEQ ID NO: 111 - protein, 7 aa, synthetic construct
DNNKRAS

SEQ ID NO: 112 - protein, 7 aa, synthetic construct
DNNKRAS

SEQ ID NO: 113 - protein, 7 aa, synthetic construct
DNNKRAS

SEQ ID NO: 114 - protein, 10 aa, synthetic construct
LLIYDNNKRA

SEQ ID NO: 115 - skipped

SEQ ID NO: 116 - protein, 11 aa, synthetic construct
GTWDNSLSAVV

SEQ ID NO: 117 - protein, 11 aa, synthetic construct
GTWDNSLSAVV

SEQ ID NO: 118 - protein, 11 aa, synthetic construct
GTWDNSLSAVV

SEQ ID NO: 119 - protein, 10 aa, synthetic construct
GTWDNSLSAV

SEQ ID NO: 120 - protein, 11 aa, synthetic construct
GTWDNSLSAVV

SEQ ID NO: 121 - protein, 7 aa, synthetic construct
GYTFTSY

SEQ ID NO: 122 - protein, 10 aa, synthetic construct
GYTFTSYAMH

SEQ ID NO: 123 - protein, 5 aa, synthetic construct
SYAMH

SEQ ID NO: 124 - protein, 6 aa, synthetic construct
TSYAMH

SEQ ID NO: 125 - protein, 8 aa, synthetic construct
GYTFTSYA

SEQ ID NO: 126 - protein, 6 aa, synthetic construct
NAGNGN

SEQ ID NO: 127 - protein, 10 aa, synthetic construct
WINAGNGNTK

SEQ ID NO: 128 - protein, 17 aa, synthetic construct
WINAGNGNTKYSQKFQG

SEQ ID NO: 129 - protein, 13 aa, synthetic construct
WMGWINAGNGNTK

SEQ ID NO: 130 - protein, 8 aa, synthetic construct
INAGNGNT

SEQ ID NO: 131 - protein, 9 aa, synthetic construct
GRELLTFDY

SEQ ID NO: 132 - protein, 9 aa, synthetic construct
GRELLTFDY

SEQ ID NO: 133 - protein, 9 aa, synthetic construct
GRELLTFDY

SEQ ID NO: 134 - protein, 10 aa, synthetic construct
ARGRELLTFD

SEQ ID NO: 135 - protein, 11 aa, synthetic construct
ARGRELLTFDY

SEQ ID NO: 136 - protein, 16 aa, synthetic construct
RSSQSLLHSNGYNYLD

SEQ ID NO: 137 - protein, 16 aa, synthetic construct
RSSQSLLHSNGYNYLD

SEQ ID NO: 138 - protein, 16 aa, synthetic construct
RSSQSLLHSNGYNYLD

SEQ ID NO: 139 - protein, 12 aa, synthetic construct
LHSNGYNYLDWY

SEQ ID NO: 140 - protein, 11 aa, synthetic construct
QSLLHSNGYNY

SEQ ID NO: 141 - protein, 7 aa, synthetic construct
LGSNRAS

SEQ ID NO: 142 - protein, 7 aa, synthetic construct
LGSNRAS

SEQ ID NO: 143 - protein, 7 aa, synthetic construct
LGSNRAS

SEQ ID NO: 144 - protein, 10 aa, synthetic construct
LLIYLGSNRA

SEQ ID NO: 145 - skipped

SEQ ID NO: 146 - protein, 9 aa, synthetic construct
MQALQTPLT

SEQ ID NO: 147 - protein, 9 aa, synthetic construct
MQALQTPLT

SEQ ID NO: 148 - protein, 9 aa, synthetic construct
MQALQTPLT

SEQ ID NO: 149 - protein, 8 aa, synthetic construct
MQALQTPL

SEQ ID NO: 150 - protein, 9 aa, synthetic construct
MQALQTPLT

SEQ ID NO: 151 - protein, 9 aa, synthetic construct
GGSISSGGY

SEQ ID NO: 152 - protein, 12 aa, synthetic construct
GGSISSGGYYWS

SEQ ID NO: 153 - protein, 7 aa, synthetic construct
SGGYYWS

SEQ ID NO: 154 - protein, 8 aa, synthetic construct
SSGGYYWS

SEQ ID NO: 155 - protein, 10 aa, synthetic construct
GGSISSGGYY

SEQ ID NO: 156 - protein, 5 aa, synthetic construct
YYSGS

SEQ ID NO: 157 - protein, 9 aa, synthetic construct
YIYYSGSTY

SEQ ID NO: 158 - protein, 16 aa, synthetic construct
YIYYSGSTYYNPSLKS

SEQ ID NO: 159 - protein, 12 aa, synthetic construct
WIGYIYYSGSTY

SEQ ID NO: 160 - protein, 7 aa, synthetic construct
IYYSGST

SEQ ID NO: 161 - protein, 11 aa, synthetic construct
DAPLRIGAFDI

SEQ ID NO: 162 - protein, 11 aa, synthetic construct
DAPLRIGAFDI

SEQ ID NO: 163 - protein, 11 aa, synthetic construct
DAPLRIGAFDI

SEQ ID NO: 164 - protein, 12 aa, synthetic construct
ARDAPLRIGAFD

SEQ ID NO: 165 - protein, 13 aa, synthetic construct
ARDAPLRIGAFDI

SEQ ID NO: 166 - protein, 13 aa, synthetic construct
SGSSSNIGNNYVS

SEQ ID NO: 167 - protein, 13 aa, synthetic construct
SGSSSNIGNNYVS

SEQ ID NO: 168 - protein, 13 aa, synthetic construct
SGSSSNIGNNYVS

SEQ ID NO: 169 - protein, 9 aa, synthetic construct
IGNNYVSWY

SEQ ID NO: 170 - protein, 8 aa, synthetic construct
SSNIGNNY

SEQ ID NO: 171 - protein, 7 aa, synthetic construct
DNNKRPS

SEQ ID NO: 172 - protein, 7 aa, synthetic construct
DNNKRPS

SEQ ID NO: 173 - protein, 7 aa, synthetic construct
DNNKRPS

SEQ ID NO: 174 - protein, 10 aa, synthetic construct
LLIYDNNKRP

SEQ ID NO: 175 - skipped

SEQ ID NO: 176 - protein, 11 aa, synthetic construct
GTWDSSLSAGV

SEQ ID NO: 177 - protein, 11 aa, synthetic construct
GTWDSSLSAGV

SEQ ID NO: 178 - protein, 11 aa, synthetic construct
GTWDSSLSAGV

SEQ ID NO: 179 - protein, 10 aa, synthetic construct
GTWDSSLSAG

SEQ ID NO: 180 - protein, 11 aa, synthetic construct
GTWDSSLSAGV

SEQ ID NO: 181 - protein, 7 aa, synthetic construct
GFTFSSY

SEQ ID NO: 182 - protein, 10 aa, synthetic construct
GFTFSSYAMH

SEQ ID NO: 183 - protein, 5 aa, synthetic construct
SYAMH

SEQ ID NO: 184 - protein, 6 aa, synthetic construct
SSYAMH

SEQ ID NO: 185 - protein, 8 aa, synthetic construct
GFTFSSYA

SEQ ID NO: 186 - protein, 6 aa, synthetic construct
SYDGSN

SEQ ID NO: 187 - protein, 10 aa, synthetic construct
VISYDGSNKY

SEQ ID NO: 188 - protein, 17 aa, synthetic construct
VISYDGSNKYYADSVKG

SEQ ID NO: 189 - protein, 13 aa, synthetic construct
WVAVISYDGSNKY

SEQ ID NO: 190 - protein, 8 aa, synthetic construct
ISYDGSNK

SEQ ID NO: 191 - protein, 12 aa, synthetic construct
PVLRYFDWLPNY

SEQ ID NO: 192 - protein, 12 aa, synthetic construct
PVLRYFDWLPNY

SEQ ID NO: 193 - protein, 12 aa, synthetic construct
PVLRYFDWLPNY

SEQ ID NO: 194 - protein, 13 aa, synthetic construct
ASPVLRYFDWLPN

SEQ ID NO: 195 - protein, 14 aa, synthetic construct
ASPVLRYFDWLPNY

SEQ ID NO: 196 - protein, 16 aa, synthetic construct
RSSQSLVYSDGNTYLN

SEQ ID NO: 197 - protein, 16 aa, synthetic construct
RSSQSLVYSDGNTYLN

SEQ ID NO: 198 - protein, 16 aa, synthetic construct
RSSQSLVYSDGNTYLN

SEQ ID NO: 199 - protein, 12 aa, synthetic construct
VYSDGNTYLNWF

SEQ ID NO: 200 - protein, 11 aa, synthetic construct
QSLVYSDGNTY

SEQ ID NO: 201 - protein, 7 aa, synthetic construct
KVSNRDS

SEQ ID NO: 202 - protein, 7 aa, synthetic construct
KVSNRDS

SEQ ID NO: 203 - protein, 7 aa, synthetic construct
KVSNRDS

SEQ ID NO: 204 - protein, 10 aa, synthetic construct
RLIYKVSNRD

SEQ ID NO: 205 - skipped

SEQ ID NO: 206 - protein, 9 aa, synthetic construct
MQGTHWPPA

SEQ ID NO: 207 - protein, 9 aa, synthetic construct
MQGTHWPPA

SEQ ID NO: 208 - protein, 9 aa, synthetic construct
MQGTHWPPA

SEQ ID NO: 209 - protein, 8 aa, synthetic construct
MQGTHWPP

SEQ ID NO: 210 - protein, 9 aa, synthetic construct
MQGTHWPPA

SEQ ID NO: 211 - protein, 7 aa, synthetic construct
GYTFTSY

SEQ ID NO: 212 - protein, 10 aa, synthetic construct
GYTFTSYGIS

SEQ ID NO: 213 - protein, 5 aa, synthetic construct
SYGIS

SEQ ID NO: 214 - protein, 6 aa, synthetic construct
TSYGIS

SEQ ID NO: 215 - protein, 8 aa, synthetic construct
GYTFTSYG

SEQ ID NO: 216 - protein, 6 aa, synthetic construct
SAYNGN

SEQ ID NO: 217 - protein, 10 aa, synthetic construct
WISAYNGNTN

SEQ ID NO: 218 - protein, 17 aa, synthetic construct
WISAYNGNTNYAQKLQG

SEQ ID NO: 219 - protein, 13 aa, synthetic construct
WMGWISAYNGNTN

SEQ ID NO: 220 - protein, 8 aa, synthetic construct
ISAYNGNT

SEQ ID NO: 221 - protein, 11 aa, synthetic construct
DVGYSYGIFDY

SEQ ID NO: 222 - protein, 11 aa, synthetic construct
DVGYSYGIFDY

SEQ ID NO: 223 - protein, 11 aa, synthetic construct
DVGYSYGIFDY

SEQ ID NO: 224 - protein, 12 aa, synthetic construct
ARDVGYSYGIFD

SEQ ID NO: 225 - protein, 13 aa, synthetic construct
ARDVGYSYGIFDY

SEQ ID NO: 226 - protein, 16 aa, synthetic construct
RSSQSLLHSNGYNYLD

SEQ ID NO: 227 - protein, 16 aa, synthetic construct
RSSQSLLHSNGYNYLD

SEQ ID NO: 228 - protein, 16 aa, synthetic construct
RSSQSLLHSNGYNYLD

SEQ ID NO: 229 - protein, 12 aa, synthetic construct
LHSNGYNYLDWY

SEQ ID NO: 230 - protein, 11 aa, synthetic construct
QSLLHSNGYNY

SEQ ID NO: 231 - protein, 7 aa, synthetic construct
LGSNRAS

SEQ ID NO: 232 - protein, 7 aa, synthetic construct
LGSNRAS

SEQ ID NO: 233 - protein, 7 aa, synthetic construct
LGSNRAS

SEQ ID NO: 234 - protein, 10 aa, synthetic construct
LLIYLGSNRA

SEQ ID NO: 235 - skipped

SEQ ID NO: 236 - protein, 9 aa, synthetic construct
MQALQTPYT

SEQ ID NO: 237 - protein, 9 aa, synthetic construct
MQALQTPYT

SEQ ID NO: 238 - protein, 9 aa, synthetic construct
MQALQTPYT

SEQ ID NO: 239 - protein, 8 aa, synthetic construct
MQALQTPY

SEQ ID NO: 240 - protein, 9 aa, synthetic construct
MQALQTPYT

SEQ ID NO: 241 - protein, 7 aa, synthetic construct
GYTFTSY

SEQ ID NO: 242 - protein, 10 aa, synthetic construct
GYTFTSYYMH

SEQ ID NO: 243 - protein, 5 aa, synthetic construct
SYYMH

SEQ ID NO: 244 - protein, 6 aa, synthetic construct
TSYYMH

SEQ ID NO: 245 - protein, 8 aa, synthetic construct
GYTFTSYY

SEQ ID NO: 246 - protein, 6 aa, synthetic construct
NPSGGS

SEQ ID NO: 247 - protein, 10 aa, synthetic construct
IINPSGGSTS

SEQ ID NO: 248 - protein, 17 aa, synthetic construct
IINPSGGSTSYAQKFQG

SEQ ID NO: 249 - protein, 13 aa, synthetic construct
WMGIINPSGGSTS

SEQ ID NO: 250 - protein, 8 aa, synthetic construct
INPSGGST

SEQ ID NO: 251 - protein, 9 aa, synthetic construct
APGKFPLDY

SEQ ID NO: 252 - protein, 9 aa, synthetic construct
APGKFPLDY

SEQ ID NO: 253 - protein, 9 aa, synthetic construct
APGKFPLDY

SEQ ID NO: 254 - protein, 10 aa, synthetic construct
ARAPGKFPLD

SEQ ID NO: 255 - protein, 11 aa, synthetic construct
ARAPGKFPLDY

SEQ ID NO: 256 - protein, 16 aa, synthetic construct
RSSQSLLHSNGYNYLD

SEQ ID NO: 257 - protein, 16 aa, synthetic construct
RSSQSLLHSNGYNYLD

SEQ ID NO: 258 - protein, 16 aa, synthetic construct
RSSQSLLHSNGYNYLD

SEQ ID NO: 259 - protein, 12 aa, synthetic construct
LHSNGYNYLDWY

SEQ ID NO: 260 - protein, 11 aa, synthetic construct
QSLLHSNGYNY

SEQ ID NO: 261 - protein, 7 aa, synthetic construct
LGSNRAS

SEQ ID NO: 262 - protein, 7 aa, synthetic construct
LGSNRAS

SEQ ID NO: 263 - protein, 7 aa, synthetic construct
LGSNRAS

SEQ ID NO: 264 - protein, 10 aa, synthetic construct
LLIYLGSNRA

SEQ ID NO: 265 - skipped

SEQ ID NO: 266 - protein, 9 aa, synthetic construct
MQALQTPRT

SEQ ID NO: 267 - protein, 9 aa, synthetic construct
MQALQTPRT

SEQ ID NO: 268 - protein, 9 aa, synthetic construct
MQALQTPRT

SEQ ID NO: 269 - protein, 8 aa, synthetic construct
MQALQTPR

SEQ ID NO: 270 - protein, 9 aa, synthetic construct
MQALQTPRT

SEQ ID NO: 271 - protein, 7 aa, synthetic construct
GFTFSSY

SEQ ID NO: 272 - protein, 10 aa, synthetic construct
GFTFSSYAMS

SEQ ID NO: 273 - protein, 5 aa, synthetic construct
SYAMS

SEQ ID NO: 274 - protein, 6 aa, synthetic construct
SSYAMS

SEQ ID NO: 275 - protein, 8 aa, synthetic construct
GFTFSSYA

SEQ ID NO: 276 - protein, 6 aa, synthetic construct
SGSGGS

SEQ ID NO: 277 - protein, 10 aa, synthetic construct
AISGSGGSTY

SEQ ID NO: 278 - protein, 17 aa, synthetic construct
AISGSGGSTYYADSVKG

SEQ ID NO: 279 - protein, 13 aa, synthetic construct
WVSAISGSGGSTY

SEQ ID NO: 280 - protein, 8 aa, synthetic construct
ISGSGGST

SEQ ID NO: 281 - protein, 16 aa, synthetic construct
GGFWGSYRYWSAAFDI

SEQ ID NO: 282 - protein, 16 aa, synthetic construct
GGFWGSYRYWSAAFDI

SEQ ID NO: 283 - protein, 16 aa, synthetic construct
GGFWGSYRYWSAAFDI

SEQ ID NO: 284 - protein, 17 aa, synthetic construct
ARGGFWGSYRYWSAAFD

SEQ ID NO: 285 - protein, 18 aa, synthetic construct
ARGGFWGSYRYWSAAFDI

SEQ ID NO: 286 - protein, 16 aa, synthetic construct
RSSQSLLHSNGYNYLD

SEQ ID NO: 287 - protein, 16 aa, synthetic construct
RSSQSLLHSNGYNYLD

SEQ ID NO: 288 - protein, 16 aa, synthetic construct
RSSQSLLHSNGYNYLD

SEQ ID NO: 289 - protein, 12 aa, synthetic construct
LHSNGYNYLDWY

SEQ ID NO: 290 - protein, 11 aa, synthetic construct
QSLLHSNGYNY

SEQ ID NO: 291 - protein, 7 aa, synthetic construct
LGSNRAS

SEQ ID NO: 292 - protein, 7 aa, synthetic construct
LGSNRAS

SEQ ID NO: 293 - protein, 7 aa, synthetic construct
LGSNRAS

SEQ ID NO: 294 - protein, 10 aa, synthetic construct
LLIYLGSNRA

SEQ ID NO: 295 - skipped

SEQ ID NO: 296 - protein, 9 aa, synthetic construct
MQALQTPPT

SEQ ID NO: 297 - protein, 9 aa, synthetic construct
MQALQTPPT

SEQ ID NO: 298 - protein, 9 aa, synthetic construct
MQALQTPPT

SEQ ID NO: 299 - protein, 8 aa, synthetic construct
MQALQTPP

SEQ ID NO: 300 - protein, 9 aa, synthetic construct
MQALQTPPT

SEQ ID NO: 301 - protein, 7 aa, synthetic construct
GYTFTSY

SEQ ID NO: 302 - protein, 10 aa, synthetic construct
GYTFTSYGIT

SEQ ID NO: 303 - protein, 5 aa, synthetic construct
SYGIT

SEQ ID NO: 304 - protein, 6 aa, synthetic construct
TSYGIT

SEQ ID NO: 305 - protein, 8 aa, synthetic construct
GYTFTSYG

SEQ ID NO: 306 - protein, 6 aa, synthetic construct
SAYNGN

SEQ ID NO: 307 - protein, 10 aa, synthetic construct
WISAYNGNTN

SEQ ID NO: 308 - protein, 17 aa, synthetic construct
WISAYNGNTNYAQKLQG

SEQ ID NO: 309 - protein, 13 aa, synthetic construct
WMGWISAYNGNTN

SEQ ID NO: 310 - protein, 8 aa, synthetic construct
ISAYNGNT

SEQ ID NO: 311 - protein, 14 aa, synthetic construct
HHPTGGSATIVFDY

SEQ ID NO: 312 - protein, 14 aa, synthetic construct
HHPTGGSATIVFDY

SEQ ID NO: 313 - protein, 14 aa, synthetic construct
HHPTGGSATIVFDY

SEQ ID NO: 314 - protein, 15 aa, synthetic construct
ARHHPTGGSATIVFD

SEQ ID NO: 315 - protein, 16 aa, synthetic construct
ARHHPTGGSATIVFDY

SEQ ID NO: 316 - protein, 16 aa, synthetic construct
RSSQSLLHSNGYNYLD

SEQ ID NO: 317 - protein, 16 aa, synthetic construct
RSSQSLLHSNGYNYLD

SEQ ID NO: 318 - protein, 16 aa, synthetic construct
RSSQSLLHSNGYNYLD

SEQ ID NO: 319 - protein, 12 aa, synthetic construct
LHSNGYNYLDWY

SEQ ID NO: 320 - protein, 11 aa, synthetic construct
QSLLHSNGYNY

SEQ ID NO: 321 - protein, 7 aa, synthetic construct
LGSNRAS

SEQ ID NO: 322 - protein, 7 aa, synthetic construct
LGSNRAS

SEQ ID NO: 323 - protein, 7 aa, synthetic construct
LGSNRAS

SEQ ID NO: 324 - protein, 10 aa, synthetic construct
LLIYLGSNRA

SEQ ID NO: 325 - skipped

SEQ ID NO: 326 - protein, 9 aa, synthetic construct
MQALQTPLT

SEQ ID NO: 327 - protein, 9 aa, synthetic construct
MQALQTPLT

SEQ ID NO: 328 - protein, 9 aa, synthetic construct
MQALQTPLT

SEQ ID NO: 329 - protein, 8 aa, synthetic construct
MQALQTPL

SEQ ID NO: 330 - protein, 9 aa, synthetic construct
MQALQTPLT

SEQ ID NO: 331 - protein, 7 aa, synthetic construct
GYTFTSY

SEQ ID NO: 332 - protein, 10 aa, synthetic construct
GYTFTSYYMH

SEQ ID NO: 333 - protein, 5 aa, synthetic construct
SYYMH

SEQ ID NO: 334 - protein, 6 aa, synthetic construct
TSYYMH

SEQ ID NO: 335 - protein, 8 aa, synthetic construct
GYTFTSYY

SEQ ID NO: 336 - protein, 6 aa, synthetic construct
NPSGGS

SEQ ID NO: 337 - protein, 10 aa, synthetic construct
IINPSGGSTS

SEQ ID NO: 338 - protein, 17 aa, synthetic construct
IINPSGGSTSYAQKFQG

SEQ ID NO: 339 - protein, 13 aa, synthetic construct
WMGIINPSGGSTS

SEQ ID NO: 340 - protein, 8 aa, synthetic construct
INPSGGST

SEQ ID NO: 341 - protein, 9 aa, synthetic construct
APGKFPLDY

SEQ ID NO: 342 - protein, 9 aa, synthetic construct
APGKFPLDY

SEQ ID NO: 343 - protein, 9 aa, synthetic construct
APGKFPLDY

SEQ ID NO: 344 - protein, 10 aa, synthetic construct
ARAPGKFPLD

SEQ ID NO: 345 - protein, 11 aa, synthetic construct
ARAPGKFPLDY

SEQ ID NO: 346 - protein, 16 aa, synthetic construct
RSSQSLLHSNGYNYLD

SEQ ID NO: 347 - protein, 16 aa, synthetic construct
RSSQSLLHSNGYNYLD

SEQ ID NO: 348 - protein, 16 aa, synthetic construct
RSSQSLLHSNGYNYLD

SEQ ID NO: 349 - protein, 12 aa, synthetic construct
LHSNGYNYLDWY

SEQ ID NO: 350 - protein, 11 aa, synthetic construct
QSLLHSNGYNY

SEQ ID NO: 351 - protein, 7 aa, synthetic construct
LGSNRAS

SEQ ID NO: 352 - protein, 7 aa, synthetic construct
LGSNRAS

SEQ ID NO: 353 - protein, 7 aa, synthetic construct
LGSNRAS

SEQ ID NO: 354 - protein, 10 aa, synthetic construct
LLIYLGSNRA

SEQ ID NO: 355 - skipped

SEQ ID NO: 356 - protein, 8 aa, synthetic construct
MQALQTPG

SEQ ID NO: 357 - protein, 8 aa, synthetic construct
MQALQTPG

SEQ ID NO: 358 - protein, 8 aa, synthetic construct
MQALQTPG

SEQ ID NO: 359 - protein, 7 aa, synthetic construct
MQALQTP

SEQ ID NO: 360 - protein, 8 aa, synthetic construct
MQALQTPG

SEQ ID NO: 361 - protein, 7 aa, synthetic construct
GYTFTSY

SEQ ID NO: 362 - protein, 10 aa, synthetic construct
GYTFTSYYMH

SEQ ID NO: 363 - protein, 5 aa, synthetic construct
SYYMH

SEQ ID NO: 364 - protein, 6 aa, synthetic construct
TSYYMH

SEQ ID NO: 365 - protein, 8 aa, synthetic construct
GYTFTSYY

SEQ ID NO: 366 - protein, 6 aa, synthetic construct
NPSGGS

SEQ ID NO: 367 - protein, 10 aa, synthetic construct
IINPSGGSTS

SEQ ID NO: 368 - protein, 17 aa, synthetic construct
IINPSGGSTSYAQKFQG

SEQ ID NO: 369 - protein, 13 aa, synthetic construct
WMGIINPSGGSTS

SEQ ID NO: 370 - protein, 8 aa, synthetic construct
INPSGGST

SEQ ID NO: 371 - protein, 9 aa, synthetic construct
APGKFPLDY

SEQ ID NO: 372 - protein, 9 aa, synthetic construct
APGKFPLDY

SEQ ID NO: 373 - protein, 9 aa, synthetic construct
APGKFPLDY

SEQ ID NO: 374 - protein, 10 aa, synthetic construct
ARAPGKFPLD

SEQ ID NO: 375 - protein, 11 aa, synthetic construct
ARAPGKFPLDY

SEQ ID NO: 376 - protein, 16 aa, synthetic construct
RSSQSLLHSNGYNYLD

SEQ ID NO: 377 - protein, 16 aa, synthetic construct
RSSQSLLHSNGYNYLD

SEQ ID NO: 378 - protein, 16 aa, synthetic construct
RSSQSLLHSNGYNYLD

SEQ ID NO: 379 - protein, 12 aa, synthetic construct
LHSNGYNYLDWY

SEQ ID NO: 380 - protein, 11 aa, synthetic construct
QSLLHSNGYNY

SEQ ID NO: 381 - protein, 7 aa, synthetic construct
LGSNRAS

SEQ ID NO: 382 - protein, 7 aa, synthetic construct
LGSNRAS

SEQ ID NO: 383 - protein, 7 aa, synthetic construct
LGSNRAS

SEQ ID NO: 384 - protein, 10 aa, synthetic construct
LLIYLGSNRA

SEQ ID NO: 385 - skipped

SEQ ID NO: 386 - protein, 9 aa, synthetic construct
MQALQTPWT

SEQ ID NO: 387 - protein, 9 aa, synthetic construct
MQALQTPWT

SEQ ID NO: 388 - protein, 9 aa, synthetic construct
MQALQTPWT

SEQ ID NO: 389 - protein, 8 aa, synthetic construct
MQALQTPW

SEQ ID NO: 390 - protein, 9 aa, synthetic construct
MQALQTPWT

SEQ ID NO: 391 - protein, 7 aa, synthetic construct
GYSFTSY

SEQ ID NO: 392 - protein, 10 aa, synthetic construct
GYSFTSYWIG

SEQ ID NO: 393 - protein, 5 aa, synthetic construct
SYWIG

SEQ ID NO: 394 - protein, 6 aa, synthetic construct
TSYWIG

SEQ ID NO: 395 - protein, 8 aa, synthetic construct
GYSFTSYW

SEQ ID NO: 396 - protein, 6 aa, synthetic construct
YPGDSD

SEQ ID NO: 397 - protein, 10 aa, synthetic construct
IIYPGDSDTR

SEQ ID NO: 398 - protein, 17 aa, synthetic construct
IIYPGDSDTRYSPSFQG

SEQ ID NO: 399 - protein, 13 aa, synthetic construct
WMGIIYPGDSDTR

SEQ ID NO: 400 - protein, 8 aa, synthetic construct
IYPGDSDT

SEQ ID NO: 401 - protein, 13 aa, synthetic construct
AIVGATSGYWFDP

SEQ ID NO: 402 - protein, 13 aa, synthetic construct
AIVGATSGYWFDP

SEQ ID NO: 403 - protein, 13 aa, synthetic construct
AIVGATSGYWFDP

SEQ ID NO: 404 - protein, 14 aa, synthetic construct
ARAIVGATSGYWFD

SEQ ID NO: 405 - protein, 15 aa, synthetic construct
ARAIVGATSGYWFDP

SEQ ID NO: 406 - protein, 16 aa, synthetic construct
RSSQSLLHSNGYNYLD

SEQ ID NO: 407 - protein, 16 aa, synthetic construct
RSSQSLLHSNGYNYLD

SEQ ID NO: 408 - protein, 16 aa, synthetic construct
RSSQSLLHSNGYNYLD

SEQ ID NO: 409 - protein, 12 aa, synthetic construct
LHSNGYNYLDWY

SEQ ID NO: 410 - protein, 11 aa, synthetic construct
QSLLHSNGYNY

SEQ ID NO: 411 - protein, 7 aa, synthetic construct
LGSNRAS

SEQ ID NO: 412 - protein, 7 aa, synthetic construct
LGSNRAS

SEQ ID NO: 413 - protein, 7 aa, synthetic construct
LGSNRAS

SEQ ID NO: 414 - protein, 10 aa, synthetic construct
LLIYLGSNRA

SEQ ID NO: 415 - skipped

SEQ ID NO: 416 - protein, 9 aa, synthetic construct
MQALQTPLT

SEQ ID NO: 417 - protein, 9 aa, synthetic construct
MQALQTPLT

SEQ ID NO: 418 - protein, 9 aa, synthetic construct
MQALQTPLT

SEQ ID NO: 419 - protein, 8 aa, synthetic construct
MQALQTPL

SEQ ID NO: 420 - protein, 9 aa, synthetic construct
MQALQTPLT

SEQ ID NO: 421 - protein, 7 aa, synthetic construct
GFTFSSY

SEQ ID NO: 422 - protein, 10 aa, synthetic construct
GFTFSSYAMH

SEQ ID NO: 423 - protein, 5 aa, synthetic construct
SYAMH

SEQ ID NO: 424 - protein, 6 aa, synthetic construct
SSYAMH

SEQ ID NO: 425 - protein, 8 aa, synthetic construct
GFTFSSYA

SEQ ID NO: 426 - protein, 6 aa, synthetic construct
SYDGSN

SEQ ID NO: 427 - protein, 10 aa, synthetic construct
VISYDGSNKY

SEQ ID NO: 428 - protein, 17 aa, synthetic construct
VISYDGSNKYYADSVKG

SEQ ID NO: 429 - protein, 13 aa, synthetic construct
WVAVISYDGSNKY

SEQ ID NO: 430 - protein, 8 aa, synthetic construct
ISYDGSNK

SEQ ID NO: 431 - protein, 12 aa, synthetic construct
PVLRYFDWLPNY

SEQ ID NO: 432 - protein, 12 aa, synthetic construct
PVLRYFDWLPNY

SEQ ID NO: 433 - protein, 12 aa, synthetic construct
PVLRYFDWLPNY

SEQ ID NO: 434 - protein, 13 aa, synthetic construct
ASPVLRYFDWLPN

SEQ ID NO: 435 - protein, 14 aa, synthetic construct
ASPVLRYFDWLPNY

SEQ ID NO: 436 - protein, 16 aa, synthetic construct
RSSQSLVYSDGNTYLN

SEQ ID NO: 437 - protein, 16 aa, synthetic construct
RSSQSLVYSDGNTYLN

SEQ ID NO: 438 - protein, 16 aa, synthetic construct
RSSQSLVYSDGNTYLN

SEQ ID NO: 439 - protein, 12 aa, synthetic construct
VYSDGNTYLNWF

SEQ ID NO: 440 - protein, 11 aa, synthetic construct
QSLVYSDGNTY

SEQ ID NO: 441 - protein, 7 aa, synthetic construct
KVSNRDS

SEQ ID NO: 442 - protein, 7 aa, synthetic construct
KVSNRDS

SEQ ID NO: 443 - protein, 7 aa, synthetic construct
KVSNRDS

SEQ ID NO: 444 - protein, 10 aa, synthetic construct
RLIYKVSNRD

SEQ ID NO: 445 - skipped

SEQ ID NO: 446 - protein, 9 aa, synthetic construct
MQGTHWPYS

SEQ ID NO: 447 - protein, 9 aa, synthetic construct
MQGTHWPYS

SEQ ID NO: 448 - protein, 9 aa, synthetic construct
MQGTHWPYS

SEQ ID NO: 449 - protein, 8 aa, synthetic construct
MQGTHWPY

SEQ ID NO: 450 - protein, 9 aa, synthetic construct
MQGTHWPYS

SEQ ID NO: 451 - protein, 7 aa, synthetic construct
GYSFTSY

SEQ ID NO: 452 - protein, 10 aa, synthetic construct
GYSFTSYWIG

SEQ ID NO: 453 - protein, 5 aa, synthetic construct
SYWIG

SEQ ID NO: 454 - protein, 6 aa, synthetic construct
TSYWIG

SEQ ID NO: 455 - protein, 8 aa, synthetic construct
GYSFTSYW

SEQ ID NO: 456 - protein, 6 aa, synthetic construct
YPGDSD

SEQ ID NO: 457 - protein, 10 aa, synthetic construct
IIYPGDSDTR

SEQ ID NO: 458 - protein, 17 aa, synthetic construct
IIYPGDSDTRYSPSFQG

SEQ ID NO: 459 - protein, 13 aa, synthetic construct
WMGIIYPGDSDTR

SEQ ID NO: 460 - protein, 8 aa, synthetic construct
IYPGDSDT

SEQ ID NO: 461 - protein, 9 aa, synthetic construct
GHGATAFDI

SEQ ID NO: 462 - protein, 9 aa, synthetic construct
GHGATAFDI

SEQ ID NO: 463 - protein, 9 aa, synthetic construct
GHGATAFDI

SEQ ID NO: 464 - protein, 10 aa, synthetic construct
ARGHGATAFD

SEQ ID NO: 465 - protein, 11 aa, synthetic construct
ARGHGATAFDI

SEQ ID NO: 466 - protein, 16 aa, synthetic construct
RSSQSLLHSNGYNYLD

SEQ ID NO: 467 - protein, 16 aa, synthetic construct
RSSQSLLHSNGYNYLD

SEQ ID NO: 468 - protein, 16 aa, synthetic construct
RSSQSLLHSNGYNYLD

SEQ ID NO: 469 - protein, 12 aa, synthetic construct
LHSNGYNYLDWY

SEQ ID NO: 470 - protein, 11 aa, synthetic construct
QSLLHSNGYNY

SEQ ID NO: 471 - protein, 7 aa, synthetic construct
LGSNRAS

SEQ ID NO: 472 - protein, 7 aa, synthetic construct
LGSNRAS

SEQ ID NO: 473 - protein, 7 aa, synthetic construct
LGSNRAS

SEQ ID NO: 474 - protein, 10 aa, synthetic construct
LLIYLGSNRA

SEQ ID NO: 475 - skipped

SEQ ID NO: 476 - protein, 9 aa, synthetic construct
MQALQTPPT

SEQ ID NO: 477 - protein, 9 aa, synthetic construct
MQALQTPPT

SEQ ID NO: 478 - protein, 9 aa, synthetic construct
MQALQTPPT

SEQ ID NO: 479 - protein, 8 aa, synthetic construct
MQALQTPP

SEQ ID NO: 480 - protein, 9 aa, synthetic construct
MQALQTPPT

SEQ ID NO: 481 - protein, 7 aa, synthetic construct
GGTFSSY

SEQ ID NO: 482 - protein, 10 aa, synthetic construct
GGTFSSYAIS

SEQ ID NO: 483 - protein, 5 aa, synthetic construct
SYAIS

SEQ ID NO: 484 - protein, 6 aa, synthetic construct
SSYAIS

SEQ ID NO: 485 - protein, 8 aa, synthetic construct
GGTFSSYA

SEQ ID NO: 486 - protein, 6 aa, synthetic construct
IPIFGT

SEQ ID NO: 487 - protein, 10 aa, synthetic construct
GIIPIFGTAN

SEQ ID NO: 488 - protein, 17 aa, synthetic construct
GIIPIFGTANYAQKFQG

SEQ ID NO: 489 - protein, 13 aa, synthetic construct
WMGGIIPIFGTAN

SEQ ID NO: 490 - protein, 8 aa, synthetic construct
IIPIFGTA

SEQ ID NO: 491 - protein, 14 aa, synthetic construct
DRYYDSSGYYLMDP

SEQ ID NO: 492 - protein, 14 aa, synthetic construct
DRYYDSSGYYLMDP

SEQ ID NO: 493 - protein, 14 aa, synthetic construct
DRYYDSSGYYLMDP

SEQ ID NO: 494 - protein, 15 aa, synthetic construct
ARDRYYDSSGYYLMD

SEQ ID NO: 495 - protein, 16 aa, synthetic construct
ARDRYYDSSGYYLMDP

SEQ ID NO: 496 - protein, 16 aa, synthetic construct
RSSQSLVHSDGNTYLS

SEQ ID NO: 497 - protein, 16 aa, synthetic construct
RSSQSLVHSDGNTYLS

SEQ ID NO: 498 - protein, 16 aa, synthetic construct
RSSQSLVHSDGNTYLS

SEQ ID NO: 499 - protein, 12 aa, synthetic construct
VHSDGNTYLSWL

SEQ ID NO: 500 - protein, 11 aa, synthetic construct
QSLVHSDGNTY

SEQ ID NO: 501 - protein, 7 aa, synthetic construct
KISNRFS

SEQ ID NO: 502 - protein, 7 aa, synthetic construct
KISNRFS

SEQ ID NO: 503 - protein, 7 aa, synthetic construct
KISNRFS

SEQ ID NO: 504 - protein, 10 aa, synthetic construct
LLIYKISNRF

SEQ ID NO: 505 - skipped

SEQ ID NO: 506 - protein, 9 aa, synthetic construct
MQATQFPLT

SEQ ID NO: 507 - protein, 9 aa, synthetic construct
MQATQFPLT

SEQ ID NO: 508 - protein, 9 aa, synthetic construct
MQATQFPLT

SEQ ID NO: 509 - protein, 8 aa, synthetic construct
MQATQFPL

SEQ ID NO: 510 - protein, 9 aa, synthetic construct
MQATQFPLT

SEQ ID NO: 511 - protein, 7 aa, synthetic construct
GYTFTSY

SEQ ID NO: 512 - protein, 10 aa, synthetic construct
GYTFTSYYMH

SEQ ID NO: 513 - protein, 5 aa, synthetic construct
SYYMH

SEQ ID NO: 514 - protein, 6 aa, synthetic construct
TSYYMH

SEQ ID NO: 515 - protein, 8 aa, synthetic construct
GYTFTSYY

SEQ ID NO: 516 - protein, 6 aa, synthetic construct
NPSGGS

SEQ ID NO: 517 - protein, 10 aa, synthetic construct
IINPSGGSTS

SEQ ID NO: 518 - protein, 17 aa, synthetic construct
IINPSGGSTSYAQKFQG

SEQ ID NO: 519 - protein, 13 aa, synthetic construct
WMGIINPSGGSTS

SEQ ID NO: 520 - protein, 8 aa, synthetic construct
INPSGGST

SEQ ID NO: 521 - protein, 9 aa, synthetic construct
APGKFPLDY

SEQ ID NO: 522 - protein, 9 aa, synthetic construct
APGKFPLDY

SEQ ID NO: 523 - protein, 9 aa, synthetic construct
APGKFPLDY

SEQ ID NO: 524 - protein, 10 aa, synthetic construct
ARAPGKFPLD

SEQ ID NO: 525 - protein, 11 aa, synthetic construct
ARAPGKFPLDY

SEQ ID NO: 526 - protein, 16 aa, synthetic construct
RSSQSLLKSNGYNYLD

SEQ ID NO: 527 - protein, 16 aa, synthetic construct
RSSQSLLKSNGYNYLD

SEQ ID NO: 528 - protein, 16 aa, synthetic construct
RSSQSLLKSNGYNYLD

SEQ ID NO: 529 - protein, 12 aa, synthetic construct
LKSNGYNYLDWY

SEQ ID NO: 530 - protein, 11 aa, synthetic construct
QSLLKSNGYNY

SEQ ID NO: 531 - protein, 7 aa, synthetic construct
LGSNRAS

SEQ ID NO: 532 - protein, 7 aa, synthetic construct
LGSNRAS

SEQ ID NO: 533 - protein, 7 aa, synthetic construct
LGSNRAS

SEQ ID NO: 534 - protein, 10 aa, synthetic construct
LLIYLGSNRA

SEQ ID NO: 535 - skipped

SEQ ID NO: 536 - protein, 9 aa, synthetic construct
MQTLQTPYT

SEQ ID NO: 537 - protein, 9 aa, synthetic construct
MQTLQTPYT

SEQ ID NO: 538 - protein, 9 aa, synthetic construct
MQTLQTPYT

SEQ ID NO: 539 - protein, 8 aa, synthetic construct
MQTLQTPY

SEQ ID NO: 540 - protein, 9 aa, synthetic construct
MQTLQTPYT

SEQ ID NO: 541 - protein, 9 aa, synthetic construct
GGSISSGGY

SEQ ID NO: 542 - protein, 12 aa, synthetic construct
GGSISSGGYYWS

SEQ ID NO: 543 - protein, 7 aa, synthetic construct
SGGYYWS

SEQ ID NO: 544 - protein, 8 aa, synthetic construct
SSGGYYWS

SEQ ID NO: 545 - protein, 10 aa, synthetic construct
GGSISSGGYY

SEQ ID NO: 546 - protein, 5 aa, synthetic construct
YYSGS

SEQ ID NO: 547 - protein, 9 aa, synthetic construct
YIYYSGSTY

SEQ ID NO: 548 - protein, 16 aa, synthetic construct
YIYYSGSTYYNPSLKS

SEQ ID NO: 549 - protein, 12 aa, synthetic construct
WIGYIYYSGSTY

SEQ ID NO: 550 - protein, 7 aa, synthetic construct
IYYSGST

SEQ ID NO: 551 - protein, 11 aa, synthetic construct
SGAAGIPWFDP

SEQ ID NO: 552 - protein, 11 aa, synthetic construct
SGAAGIPWFDP

SEQ ID NO: 553 - protein, 11 aa, synthetic construct
SGAAGIPWFDP

SEQ ID NO: 554 - protein, 12 aa, synthetic construct
ARSGAAGIPWFD

SEQ ID NO: 555 - protein, 13 aa, synthetic construct
ARSGAAGIPWFDP

SEQ ID NO: 556 - protein, 13 aa, synthetic construct
SGSSSNIGNNYVS

SEQ ID NO: 557 - protein, 13 aa, synthetic construct
SGSSSNIGNNYVS

SEQ ID NO: 558 - protein, 13 aa, synthetic construct
SGSSSNIGNNYVS

SEQ ID NO: 559 - protein, 9 aa, synthetic construct
IGNNYVSWY

SEQ ID NO: 560 - protein, 8 aa, synthetic construct
SSNIGNNY

SEQ ID NO: 561 - protein, 7 aa, synthetic construct
ENNERPS

SEQ ID NO: 562 - protein, 7 aa, synthetic construct
ENNERPS

SEQ ID NO: 563 - protein, 7 aa, synthetic construct
ENNERPS

SEQ ID NO: 564 - protein, 10 aa, synthetic construct
LLIYENNERP

SEQ ID NO: 565 - skipped

SEQ ID NO: 566 - protein, 11 aa, synthetic construct
GTWDSSLSAVV

SEQ ID NO: 567 - protein, 11 aa, synthetic construct
GTWDSSLSAVV

SEQ ID NO: 568 - protein, 11 aa, synthetic construct
GTWDSSLSAVV

SEQ ID NO: 569 - protein, 10 aa, synthetic construct
GTWDSSLSAV

SEQ ID NO: 570 - protein, 11 aa, synthetic construct
GTWDSSLSAVV

SEQ ID NO: 571 - protein, 7 aa, synthetic construct
GYTFTSY

SEQ ID NO: 572 - protein, 10 aa, synthetic construct
GYTFTSYGIS

SEQ ID NO: 573 - protein, 5 aa, synthetic construct
SYGIS

SEQ ID NO: 574 - protein, 6 aa, synthetic construct
TSYGIS

SEQ ID NO: 575 - protein, 8 aa, synthetic construct
GYTFTSYG

SEQ ID NO: 576 - protein, 6 aa, synthetic construct
SAYNGN

SEQ ID NO: 577 - protein, 10 aa, synthetic construct
WISAYNGNTN

SEQ ID NO: 578 - protein, 17 aa, synthetic construct
WISAYNGNTNYAQKLQG

SEQ ID NO: 579 - protein, 13 aa, synthetic construct
WMGWISAYNGNTN

SEQ ID NO: 580 - protein, 8 aa, synthetic construct
ISAYNGNT

SEQ ID NO: 581 - protein, 9 aa, synthetic construct
GPRDGYNDY

SEQ ID NO: 582 - protein, 9 aa, synthetic construct
GPRDGYNDY

SEQ ID NO: 583 - protein, 9 aa, synthetic construct
GPRDGYNDY

SEQ ID NO: 584 - protein, 10 aa, synthetic construct
ARGPRDGYND

SEQ ID NO: 585 - protein, 11 aa, synthetic construct
ARGPRDGYNDY

SEQ ID NO: 586 - protein, 16 aa, synthetic construct
RSSQSLLHSNGYNYLN

SEQ ID NO: 587 - protein, 16 aa, synthetic construct
RSSQSLLHSNGYNYLN

SEQ ID NO: 588 - protein, 16 aa, synthetic construct
RSSQSLLHSNGYNYLN

SEQ ID NO: 589 - protein, 12 aa, synthetic construct
LHSNGYNYLNWY

SEQ ID NO: 590 - protein, 11 aa, synthetic construct
QSLLHSNGYNY

SEQ ID NO: 591 - protein, 7 aa, synthetic construct
LGSNRAS

SEQ ID NO: 592 - protein, 7 aa, synthetic construct
LGSNRAS

SEQ ID NO: 593 - protein, 7 aa, synthetic construct
LGSNRAS

SEQ ID NO: 594 - protein, 10 aa, synthetic construct
LLIYLGSNRA

SEQ ID NO: 595 - skipped

SEQ ID NO: 596 - protein, 9 aa, synthetic construct
MQPLQTPLT

SEQ ID NO: 597 - protein, 9 aa, synthetic construct
MQPLQTPLT

SEQ ID NO: 598 - protein, 9 aa, synthetic construct
MQPLQTPLT

SEQ ID NO: 599 - protein, 8 aa, synthetic construct
MQPLQTPL

SEQ ID NO: 600 - protein, 9 aa, synthetic construct
MQPLQTPLT

SEQ ID NO: 601 - protein, 7 aa, synthetic construct
GYTFTSY

SEQ ID NO: 602 - protein, 10 aa, synthetic construct
GYTFTSYGIS

SEQ ID NO: 603 - protein, 5 aa, synthetic construct
SYGIS

SEQ ID NO: 604 - protein, 6 aa, synthetic construct
TSYGIS

SEQ ID NO: 605 - protein, 8 aa, synthetic construct
GYTFTSYG

SEQ ID NO: 606 - protein, 6 aa, synthetic construct
SAYNGN

SEQ ID NO: 607 - protein, 10 aa, synthetic construct
WISAYNGNTN

SEQ ID NO: 608 - protein, 17 aa, synthetic construct
WISAYNGNTNYAQKLQG

SEQ ID NO: 609 - protein, 13 aa, synthetic construct
WMGWISAYNGNTN

SEQ ID NO: 610 - protein, 8 aa, synthetic construct
ISAYNGNT

SEQ ID NO: 611 - protein, 9 aa, synthetic construct
MAGWELIDP

SEQ ID NO: 612 - protein, 9 aa, synthetic construct
MAGWELIDP

SEQ ID NO: 613 - protein, 9 aa, synthetic construct
MAGWELIDP

SEQ ID NO: 614 - protein, 10 aa, synthetic construct
ARMAGWELID

SEQ ID NO: 615 - protein, 11 aa, synthetic construct
ARMAGWELIDP

SEQ ID NO: 616 - protein, 16 aa, synthetic construct
RSSQSLLHSNGYNYLD

SEQ ID NO: 617 - protein, 16 aa, synthetic construct
RSSQSLLHSNGYNYLD

SEQ ID NO: 618 - protein, 16 aa, synthetic construct
RSSQSLLHSNGYNYLD

SEQ ID NO: 619 - protein, 12 aa, synthetic construct
LHSNGYNYLDWY

SEQ ID NO: 620 - protein, 11 aa, synthetic construct
QSLLHSNGYNY

SEQ ID NO: 621 - protein, 7 aa, synthetic construct
LGSNRAS

SEQ ID NO: 622 - protein, 7 aa, synthetic construct
LGSNRAS

SEQ ID NO: 623 - protein, 7 aa, synthetic construct
LGSNRAS

SEQ ID NO: 624 - protein, 10 aa, synthetic construct
LLIYLGSNRA

SEQ ID NO: 625 - skipped

SEQ ID NO: 626 - protein, 9 aa, synthetic construct
MQALQTPYS

SEQ ID NO: 627 - protein, 9 aa, synthetic construct
MQALQTPYS

SEQ ID NO: 628 - protein, 9 aa, synthetic construct
MQALQTPYS

SEQ ID NO: 629 - protein, 8 aa, synthetic construct
MQALQTPY

SEQ ID NO: 630 - protein, 9 aa, synthetic construct
MQALQTPYS

SEQ ID NO: 631 - protein, 7 aa, synthetic construct
GYSFTSY

SEQ ID NO: 632 - protein, 10 aa, synthetic construct
GYSFTSYWIG

SEQ ID NO: 633 - protein, 5 aa, synthetic construct
SYWIG

SEQ ID NO: 634 - protein, 6 aa, synthetic construct
TSYWIG

SEQ ID NO: 635 - protein, 8 aa, synthetic construct
GYSFTSYW

SEQ ID NO: 636 - protein, 6 aa, synthetic construct
YPGDSD

SEQ ID NO: 637 - protein, 10 aa, synthetic construct
IIYPGDSDTR

SEQ ID NO: 638 - protein, 17 aa, synthetic construct
IIYPGDSDTRYSPSFQG

SEQ ID NO: 639 - protein, 13 aa, synthetic construct
WMGIIYPGDSDTR

SEQ ID NO: 640 - protein, 8 aa, synthetic construct
IYPGDSDT

SEQ ID NO: 641 - protein, 9 aa, synthetic construct
GVGSVVFDY

SEQ ID NO: 642 - protein, 9 aa, synthetic construct
GVGSVVFDY

SEQ ID NO: 643 - protein, 9 aa, synthetic construct
GVGSVVFDY

SEQ ID NO: 644 - protein, 10 aa, synthetic construct
ARGVGSVVFD

SEQ ID NO: 645 - protein, 10 aa, synthetic construct
ARGVGSVVFD

SEQ ID NO: 646 - protein, 16 aa, synthetic construct
RSSQSLLHSNGYNYLD

SEQ ID NO: 647 - protein, 16 aa, synthetic construct
RSSQSLLHSNGYNYLD

SEQ ID NO: 648 - protein, 16 aa, synthetic construct
RSSQSLLHSNGYNYLD

SEQ ID NO: 649 - protein, 12 aa, synthetic construct
LHSNGYNYLDWY

SEQ ID NO: 650 - protein, 11 aa, synthetic construct
QSLLHSNGYNY

SEQ ID NO: 651 - protein, 7 aa, synthetic construct
LGSNRAS

SEQ ID NO: 652 - protein, 7 aa, synthetic construct
LGSNRAS

SEQ ID NO: 653 - protein, 7 aa, synthetic construct
LGSNRAS

SEQ ID NO: 654 - protein, 10 aa, synthetic construct
LLIYLGSNRA

SEQ ID NO: 655 - skipped

SEQ ID NO: 656 - protein, 9 aa, synthetic construct
MQALQTPPT

SEQ ID NO: 657 - protein, 9 aa, synthetic construct
MQALQTPPT

SEQ ID NO: 658 - protein, 9 aa, synthetic construct
MQALQTPPT

SEQ ID NO: 659 - protein, 8 aa, synthetic construct
MQALQTPP

SEQ ID NO: 660 - protein, 9 aa, synthetic construct
MQALQTPPT

SEQ ID NO: 661 - protein, 120 aa, synthetic construct
EVQLLESGPGLVKPSQTLSLTCTVSGGSISSGGYYWSWIRQHPGKGLEWIGYIYYSGSTYYNPSLKSRVTISVDTSKNQFSLKLSSVTAADTAVYYCARTNSRITMIADWGQGTLVTVSS

SEQ ID NO: 662 - protein, 110 aa, synthetic construct
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAGVFGGGTQLTVL

SEQ ID NO: 663 - protein, 120 aa, synthetic construct; identical to SEQ ID NO: 661

SEQ ID NO: 664 - protein, 111 aa, synthetic construct
QSVLTQPPSVSAAPGQKVTISCSGSSSNIGNNYVSWYQQLPGTAPKLLTYDNNKRPSGIPDRFSGSKSGTSATLGITGLQTGDEADYYCGTWDSSLSAGHVFGGGTKVTVL

SEQ ID NO: 665 - protein, 125 aa, synthetic construct
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSPYYYDSSGYYPLDYWGQGTLVTVSS

SEQ ID NO: 666 - protein, 112 aa, synthetic construct
DVVMTQSPLSLPVTPGEPASISCRSSQSLLHSNGYNYLDWYLQKPGQSPQLLIYLGSNRAPGVPDRFSGSGSGTDFTLKISRVEAEDVGVYYCMQALQTPLTFGPGTKLEIK

SEQ ID NO: 667 - protein, 118 aa, synthetic construct
EVQLLESGPGLVKPSQTLSLTCTVSGGSISSGDYYWSWIRQPPGKGLEWIGYIYYSGSTYYNPSLKSRVTISVDTSKNQFSLKLSSVTAADTAVYYCARGRRLLLFHWGQGTTVTVSS

SEQ ID NO: 668 - protein, 110 aa, synthetic construct
QSVLTQPPSVSAAPGQKVTISCSGSGSSIGNNYVSWYQQVPGTPPKLLIYDNNKRASGIPDRFSGSKSGTSATLGITGLQTGDEADYYCGTWDNSLSAVVFGGGTKLTVL

SEQ ID NO: 669 - protein, 118 aa, synthetic construct
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARGRELLTFDYWGQGTLVTVSS

SEQ ID NO: 670 - protein, 112 aa, synthetic construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 671 - protein, 121 aa, synthetic construct
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARDAPLRIGAFDIWGQGTMVTVSS

SEQ ID NO: 672 - protein, 110 aa, synthetic construct
QSVLTQPPSVSAAPGQKVTISCSGSSSNIGNNYVSWYQQLPGTAPKLLIYDNNKRPSGIPDRFSGSKSGTSATLGITRLQTGDEADYYCGTWDSSLSAGVFGGGTKLTVL

SEQ ID NO: 673 - protein, 121 aa, synthetic construct
EVQLLESGGGVVQPGRSLRLSCAASGFTFSSYAMHWVRQAPGKGLEWVAVISYDGSNKYYADSVKGRFTISRDNSKNTLYLQMNSLRAEDTAVYYCASPVLRYFDWLPNYWGQGTLVTVSS

SEQ ID NO: 674 - protein, 112 aa, synthetic construct
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPPAFGPGTKVDIK

SEQ ID NO: 675 - protein, 120 aa, synthetic construct
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDVGYSYGIFDYWGQGTLVTVSS

SEQ ID NO: 676 - protein, 112 aa, synthetic construct
DVVMTQSPLSLPATPGEPASISCRSSQSLLHSNGYNYLDWYLQKPGQSPQLLIYLGSNRASGVPDRFSGSGSGTDFTLKISRVEAEDVGVYYCMQALQTPYTFGQGTKVEIK

SEQ ID NO: 677 - protein, 118 aa, synthetic construct
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARAPGKFPLDYWGQGTLVTVSS

SEQ ID NO: 678 - protein, 112 aa, synthetic construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPRTFGGGTKVEIK

SEQ ID NO: 679 - protein, 125 aa, synthetic construct
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCARGGFWGSYRYWSAAFDIWGQGTMVTVSS

SEQ ID NO: 680 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPPTFGGGTKVEIK

SEQ ID NO: 681 - protein, 123 aa, synthetic construct
EVQLVESGAEVKKPGASVKVSCKASGYTFTSYGITWVRQAPGQGLEWMGWISAYNGNTNYAQKLQGRVTMTTDTSTSTAYMELRSLRSDDTAVYYCARHHPTGGSATIVFDYWGQGTLVTVSS

SEQ ID NO: 682 - protein, 111 aa, synthetic construct
DVVMTQSPLSLPVTPGEPASISCRSSQSLLHSNGYNYLDWYLQKPGQSPQLLIYLGSNRASGVPDRFRGSGSGTDFTLKISRVEAEDVGVYYCMQALQTPGFGQGTKVEIK

SEQ ID NO: 683 - protein, 118 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARAPGKFPLDYWGQGTLVTVSS

SEQ ID NO: 684 - protein, 111 aa, synthetic construct; identical to SEQ ID NO: 682

SEQ ID NO: 685 - protein, 118 aa, synthetic construct; identical to SEQ ID NO: 677

SEQ ID NO: 686 - protein, 112 aa, synthetic construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPWTFGQGTKLEIK

SEQ ID NO: 687 - protein, 122 aa, synthetic construct
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARAIVGATSGYWFDPWGQGTLVTVSS

SEQ ID NO: 688 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 689 - protein, 121 aa, synthetic construct
EVQLLESGGGVVQPGRSLRLSCAASGFTFSSYAMHWVRQAPGKGLEWVAVISYDGSNKYYADSVKGRFTISRDNSKNTLYLQMNSLRAEDTAVYYCASPVLRYFDWLPNYWGQGTTVTVSS

SEQ ID NO: 690 - protein, 112 aa, synthetic construct
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPYSFGQGTKVEIK

SEQ ID NO: 691 - protein, 118 aa, synthetic construct
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARGHGATAFDIWGQGTMVTVSS

SEQ ID NO: 692 - protein, 112 aa, synthetic construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPPTFGPGTKLEIK

SEQ ID NO: 693 - protein, 123 aa, synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARDRYYDSSGYYLMDPWGQGTLVTVSS

SEQ ID NO: 694 - protein, 112 aa, synthetic construct
DIVMTQSPLSSPVTLGQPASISCRSSQSLVHSDGNTYLSWLQQRPGQPPRLLIYKISNRFSGVPDRFSGSGAGTDFTLKISRVEAEDVGVYYCMQATQFPLTFGGGTRLEIK

SEQ ID NO: 695 - protein, 118 aa, synthetic construct; identical to SEQ ID NO: 683

SEQ ID NO: 696 - protein, 112 aa, synthetic construct
DVVMTQSPLSLPVTPGEPASISCRSSQSLLKSNGYNYLDWYLQKPGQSPQLLIYLGSNRASGVPDRFSGSGSGTDFSLKIRRVEAEDVGVYYCMQTLQTPYTFGQGTKLEIK

SEQ ID NO: 697 - protein, 121 aa, synthetic construct
QVQLVESGPGLVKPSQTLSLTCTVSGGSISSGGYYWSWIRQHPGKGLEWIGYIYYSGSTYYNPSLKSRVTISVDTSKNQFSLKLSSVTAADTAVYYCARSGAAGIPWFDPWGQGTLVTVSS

SEQ ID NO: 698 - protein, 110 aa, synthetic construct
QSVLTQPPSVSAAPGQKVTISCSGSSSNIGNNYVSWYQQLPGTAPKLLIYENNERPSGIPDRLSGSKSGTSATLAITGLQTGDEADYYCGTWDSSLSAVVFGGGTKLTVL

SEQ ID NO: 699 - protein, 118 aa, synthetic construct
QVQLVQSGAEVKKPGASVKVSCKASGYTFTSYGISWVRQAPGRGLEWMGWISAYNGNTNYAQKLQGRVTMTTDTSTSTAYMELRSLRSDDTAVYYCARGPRDGYNDYWGQGTLVTVSS

SEQ ID NO: 700 - protein, 112 aa, synthetic construct
DVVMTQSPLSLPVTPGEPASISCRSSQSLLHSNGYNYLNWYLQKPGQSPQLLIYLGSNRASGVPDRFSGSGSGTDFTLKISRVEAEDVGVYYCMQPLQTPLTFGGGTKLEIK

SEQ ID NO: 701 - protein, 118 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARMAGWELIDPWGQGTLVTVSS

SEQ ID NO: 702 - protein, 112 aa, synthetic construct
DVVMTQSPLSLPVTPGEPASISCRSSQSLLHSNGYNYLDWYLQKPGKSPQLLIYLGSNRASGVPDRFSGSGSGTDFTLKISRVEAEDVGVYYCMQALQTPYSFGQGTKVEIK

SEQ ID NO: 703 - protein, 118 aa, synthetic construct
EVQLLESGAEVKKPGESLKISCKGSGYSFTSYWIGWVRQMPGKGLEWMGIIYPGDSDTRYSPSFQGQVTISADKSISTAYLQWSSLKASDTAMYYCARGVGSVVFDYWGQGTLVTVSS

SEQ ID NO: 704 - protein, 112 aa, synthetic construct
DVVMTQSPLPLPVTPGEPASISCRSSQSLLHSNGYNYLDWYLQKPGQSPQLLIYLGSNRASGVPDRFSGSGSGTDFTLKISRVEAEDVGVYYCMQALQTPPTFGQGTKVEIK